(19) United States
(12) Patent Application Publication
(10) Pub. No.: US 2024/0081674 A1
(43) Pub. Date: Mar. 14, 2024

(54) SYSTEMS AND METHODS FOR DETECTION OF VOLATILE ORGANIC COMPOUNDS

(71) Applicant: NanoBio Detection Systems LLC, Sheridan, WY (US)

(72) Inventors: Shalini PRASAD, Allen, TX (US); Kordel Kade FRANCE, Richardson, TX (US); Ivneet Kaur BANGA, Richardson, TX (US); Anirban PAUL, Dallas, TX (US)

(21) Appl. No.: 18/187,482

(22) Filed: Mar. 21, 2023

Related U.S. Application Data
(63) Continuation of application No. 17/344,803, filed on Jun. 10, 2021, now abandoned.
(60) Provisional application No. 63/147,135, filed on Feb. 8, 2021.
(60) Provisional application No. 63/068,809, filed on Aug. 21, 2020.
(60) Provisional application No. 63/037,966, filed on Jun. 11, 2020.

(51) Int. Cl. A61B 5/08; A61B 5/00; A61B 5/097; G01N 27/413

(52) U.S. Cl. CPC A61B 5/082; A61B 5/097; A61B 5/4845; G01N 27/413

(57) ABSTRACT

Detection devices for detecting one or more target analytes such as volatile organic compounds (VOCs) may include a base and a sensor module coupleable to the base and including at least one electrochemical sensor, where the electrochemical sensor includes an electrode and an ionic liquid (e.g., room temperature ionic liquid) that is arranged on the electrode and specific to a target analyte. In some variations, at least one cavity specific to the target analyte is formed within the ionic liquid in response to the electrochemical sensor receiving an input signal.

### CROSS-REFERENCE TO RELATED APPLICATIONS

This application is a continuation of U.S. patent application Ser. No. 17/344,803, filed Jun. 10, 2021, which claims priority to U.S. Provisional Patent Application No. 63/147,135 filed Feb. 8, 2021, U.S. Patent Provisional Patent No. 63/068,809 filed Aug. 21, 2020, and U.S. Provisional Patent Application No. 63/037,966 filed Jun. 11, 2020, each of which is incorporated herein in its entirety by this reference.

### TECHNICAL FIELD

This invention relates generally to the field of analyte detection, such as detection of volatile organic compounds (VOCs).

### BACKGROUND

Volatile organic compounds (VOCs) are a class of molecules with high vapor pressure at room temperature, and many have the potential to cause damage to both environment and human health. Some VOCs are also indicators or biomarkers for disease. The ability to accurately detect the presence of VOCs may therefore be helpful in areas such as air quality monitoring, biomedical diagnostics, industrial processes, security and occupational health, etc. Conventional techniques for the detection of volatile organic compounds include mass spectrometry, gas chromatography, and ion mobility spectroscopy. However, these are bench-top techniques, which require trained personnel, large setups, costly and sophisticated equipment, and require a significant amount of time to provide results, thereby limiting their on-site applicability.

Electrochemical gas sensing techniques have been used as one on-site solution for detection of VOCs. However, conventional electrochemical gas sensors suffer from a number of drawbacks, including lack of high sensitivity or specificity toward different classes of analytes, and limited ability to detect analytes at a distance. Therefore, there is a need for new and improved systems and methods for detecting target analytes such as VOCs.

### SUMMARY

Generally, a detection device for detecting one or more volatile organic compounds (VOCs) may include a base and a sensor module that is removably coupleable to the base and contains at least one electrochemical sensor. The electrochemical sensor(s) may include an electrode and an ionic liquid that is arranged on the electrode and is specific to a target VOC. In some variations, the ionic liquid may be a room temperature ionic liquid (RTIL). The ionic liquid may, for example, include a plurality of ionic layers, wherein at least one cavity specific to the target VOC may be formed between adjacent ionic layers, such as in response to an input signal provided to the electrochemical sensor (e.g., a DC reduction potential delivered by the detection device to the electrochemical sensor). The cavity or cavities specific to the target VOC may be configured to capture the target VOC such that the captured VOC diffuses toward the electrode (e.g., for detection).

In some variations, the detection device may include one or more processors configured to detect the captured target VOC based at least in part on one or more electrical parameters (e.g., impedance, current, or both) at the electrode. The detection device may include an alarm configured to provide an alert in response to detection of the target VOC using one or more electrochemical sensors. In some variations, the detection device may include additional elements, such as a wireless communication module or a handheld housing. Additionally or alternatively, the detection device may be configured to be mounted on a surface. The detection device may be used in various applications, such as to detect a target VOC that is characteristic of an explosive, is characteristic of a drug, or is a biomarker characteristic of the health state of a user.

In some variations of the detection device, the sensor module may include a plurality of electrochemical sensors. Each of at least a portion of the plurality of electrochemical sensors may include a respective ionic liquid, where the respective ionic liquids are specific to a target VOC. The respective ionic liquids may, for instance, be specific to the same target VOC or to different target VOCs. The sensor module may additionally include elements such as one or more electrical contacts configured to conductively couple to the base, or a mouthpiece.

Generally, an electrochemical sensor for use in detecting one or more VOCs may include an electrode and a room temperature ionic liquid (RTIL) arranged over the electrode. The RTIL may include at least one cavity specific to the target VOC, such as one or more cavities formed in response to the sensor receiving an input signal. In some variations, the electrode includes one or more suitable conductive materials such as a metal (e.g., gold) or a metal alloy. In some variations, the sensor may include interdigitated electrodes.

The RTIL used in the electrochemical sensor may include any suitable room temperature ionic liquid, such as an imidazolium-based RTIL (e.g., 1-butyl-3-methylimidazolium chloride, or BMIM-Cl; 1-butyl-3-methylimidazolium tetrafluoroborate, or BMIM-BF₄; 1-ethyl-3-methylimidazolium bis-(trifluoromethanesulphonyl)imide, or EMIM-TF₂N; 1-ethyl-3-methylimidazolium tetrafluoroborate, or EMIM-BF₄; or 1-ethyl-3-methylimidazolium trifluoromethanesulfonate, or EMIM-OTf). In some variations, the RTIL may include a plurality of ionic layers (i.e., 2 or more). In some variations of the electrochemical sensor, at least one cavity is formed between adjacent ionic layers upon application of a suitable input signal (e.g., a DC reduction potential). The input signal may, for instance, correspond to a redox potential of the target VOC. A cavity may also have a size corresponding to the redox potential of the target VOC. In some variations, the cavity is configured to capture the target VOC such that the VOC diffused toward the electrode.

In some variations, the target VOC is characteristic of an explosive (e.g., 1,3-dinitrobenzene; 2,4-dinitrotoluene; 2,6-dinitrotoluene; 1-ethyl-2-nitrobenzene; 2,3-dim ethyl-2,3-dinitrobutane; sulfur dioxide; or cyclohexanone, etc.). The target VOC may also, for instance, be characteristic of C-4 or gunpowder. In some variations, the target VOC may be characteristic of the presence of one or more drugs such as fentanyl. In some variations, the target VOC is a biomarker (e.g., NOx, an aliphatic hydrocarbon (e.g., isopentane, heptane), etc.) associated with a medical condition, such as the presence of COVID-19 in a user. The electrochemical sensor may be part of a detection device.

Generally, a method for detecting one or more VOCs may include applying an input signal to an electrochemical sensor, receiving a sensor signal from the electrochemical sensor after applying the input signal, and detecting the target VOC based at least in part on the sensor signal. The electrochemical sensor may include an electrode and an ionic liquid arranged over the electrode, wherein at least one cavity specific to a target VOC is formed within the ionic liquid, such as in response to the input signal. The sensor signal may, for example, by indicative of current at the electrode. In some variations, the ionic liquid includes a room temperature ionic liquid (RTIL), and the cavity(s) present therein may be tuned to the redox potential of the target VOC.

In some variations, the method for detecting one or more VOCs may include applying an input signal to a plurality of electrochemical sensors, each including a respective electrode and respective ionic liquid arranged over the electrode. In some variations, in response to the input signal, the respective ionic liquids of at least a portion of the plurality of electrochemical sensors form cavities that are specific to the same or different target VOC(s). In some variations, the method of detecting the target VOC includes sensing the target VOC using a majority of the electrochemical sensors specific to the target VOC. In some variations, the method may include determining travel direction and/or travel speed of the target VOC, such as based on differential timing of detection of the target VOC using the electrochemical sensors specific to the target VOC. The method may include providing an alert in response to detection of the target VOC.

In some variations, the method for detecting one or more VOCs include detecting a target VOC that is characteristic of an explosive, is characteristic of a drug, or is a biomarker characteristic of the health state of a user. In some variations, the method may include detecting a target VOC emitted from a particular medium (e.g., solid, liquid, or gas).

Generally, a method for determining a health state of a user may include measuring a sensor signal of at least one electrochemical sensor receiving an aerosolized sample, detecting the target VOC based at least in part on the measured sensor signal, and determining the health state of the user based on the detected target VOC. In some variations, the as least one electrochemical sensor may include an electrode and a room temperature ionic liquid (RTIL) that is arranged on the electrode, wherein at least one cavity specific to a target volatile organic compound (VOC) is formed within the RTIL in response to the electrochemical sensor receiving an input signal. In some variations, the RTIL may include a plurality of ionic layers and the at least one cavity is formed between adjacent ionic layers. In some variations, measuring a sensor signal may include delivering an input signal to the at least one electrochemical sensor and measuring one or more electrical parameters (e.g., impedance, current, or both) at the at least one electrochemical sensor after delivering the input signal. The input signal may apply a DC reduction potential to the electrode, for example.

In some variations, the method for determining a health state of a user includes an electrochemical sensor with a cavity configured to capture the target VOC such that the target VOC diffuses toward the electrode. The method may include providing an alert in response to detection of the medical condition. In some variations, detecting the target VOC may include detecting the VOC in an aerosolized sample (e.g., breath from the user or an aerosolized body fluid such as saliva or nasal fluid). In some variations, the aerosolized sample is from a sampling device or from ambient air. The aerosolized sample may be filtered to remove particulates above a threshold size. In some variations, the method for determining a health state of a user includes an electrochemical sensor in a sensor module removably coupled to a base. The base may include a handheld unit and may be optionally configured to be mounted to a surface. If present, the sensor module may include a mouthpiece and a nozzle configured to provide for laminar flow of the aerosolized sample over the electrochemical sensor(s). In some variations of the method for determining a health state of a user, the target VOC is a biomarker characteristic of a disease (e.g., COVID-19).

Generally, a detection device for detecting one or more VOCs in breath of a user may include a base, a sensor module removably coupled to the base, and a mouthpiece configured to direct a volume of breath from the user toward the electrochemical sensor(s). In some variations, the sensor module includes at least one electrochemical sensor comprising an electrode and an ionic liquid arranged over the electrode, wherein the ionic liquid is specific to a target VOC. In some variations, the ionic liquid is a room temperature ionic liquid (RTIL). The base of the detection device may include a handheld housing.

In some variations, the detection device is configured to deliver an input signal to the electrochemical sensor, thereby forming at least one cavity specific to the target VOC within the ionic liquid. In some variations, the cavity is configured to capture the target VOC such that the captured VOC diffuses toward the electrode. In some variations, the base comprises one or more processors configured to detect the captured target VOC based at least in part on an electrical parameter (e.g., impedance, current, or both) at the electrode. The base may also include an alarm configured to provide an alert in response to detection of the target VOC using the electrochemical sensor(s). In some variations, the sensor module includes a plurality of electrochemical sensors. In some variations, at least a portion of the plurality of electrochemical sensors form cavities that are specific to the same or different target VOC(s).

In some variations of the detection device, the mouthpiece includes a tube. In some variations, the sensor module includes a nozzle configured to laminarize flow of the volume of breath over the at least one electrochemical sensor. The sensor module may also include one or more filters configured to filter particulars from the volume of breath, and additionally or alternatively, include dehumidifying elements configured to reduce moisture in the volume of breath. In some variations, the target analyte is a biomarker characteristic of a health state of the user. The health state may be a disease (e.g., COVID-19).

Generally, a detection system for detecting one or more volatile organic compounds (VOCs) in breath of a user (or other gas), may include a sensor module including at least one electrochemical sensor specific to a target VOC, and a sampling device coupleable to the sensor module or other portion of a detection device, wherein the sampling device is sealable and configured to store a volume of breath. The sensor module may, in some variations, include an electrode and an ionic liquid arranged over the electrode, where the ionic liquid may be specific to the target VOC. Furthermore, in some variations, the detection system may include an alarm configured to provide an alert in response to detection of the target VOC using the at least one electrochemical sensor.

In some variations, the sampling device may be removably coupleable to the sensor module (or other part of a detection device). The sampling device may be coupleable to the sensor module (or other part of a detection device) via a connector. The sampling device may include a compartment (e.g., for storing a volume of breath). In some variations, the compartment may be compressible.

In some variations, the sampling device may include a mouthpiece or other suitable feature for introducing breath into the sampling device. The mouthpiece may include one or more breath processing elements, such as one or more filters, and/or one or more desiccants. Furthermore, in some variations, the sampling device may include one or more one-way valves (e.g., to direct flow of breath in and/or out of the sampling device).

The detection system may, in some variations, include a base. The sensor module may be coupleable to the base, such as removably coupleable to the base. In some variations, the base may include a handheld housing.

Generally, a sampling device may include a compartment and a mouthpiece coupled to the compartment, where the sampling device may be sealable and configured to store a volume of a gas sample (e.g., breath). In some variations, the compartment may include at least one inlet and at least one outlet. In some of these variations, the mouthpiece may be coupled to an inlet of the compartment, and/or the sampling device may further include a stopper coupled to the outlet of the compartment. The stopper may be removably coupled to the outlet of the compartment.

In some variations, the sampling device may be sealable at least in part via one or more one-way valves. For example, the sampling device may include an inlet sealable with a first one-way valve, and an outlet sealable with a second one-way valve (and/or a stopper). The one or more one-way valves may include a check valve, for example.

In some variations, the compartment of the sampling device may be compressible. For example, the compartment may include a bag. In some variations, the bag may include a first sheet and a second sheet opposing the first sheet, wherein the first and second sheets are sealed together (e.g., via heat or RF welding) to form an edge or at least a portion of a perimeter of the compartment.

The mouthpiece may have any suitable shape and/or one or more gas-processing elements. For example, in some variations the mouthpiece may include a tube. Furthermore, in some variations the mouthpiece may include one or more filters and/or one or more desiccants. The mouthpiece may be coupled to the compartment via any suitable method, including, for example, heat or RF welding.

In some variations, the sampling device may be configured to removably couple to a detection device (e.g., a detection device including an electrochemical sensor for detecting a target VOC). Additionally or alternatively, in some variations, the sampling device may include one or more identification features such as a labeling region and/or a computer-readable identifier associated with the sampling device.

### BRIEF DESCRIPTION OF THE DRAWINGS

FIGS. 1A and 1B depict illustrative schematics of examples of a detection device for detecting analytes.

FIG. 2 depicts an illustrative schematic of an example of a detection system for detecting analytes.

FIG. 3 depicts an illustrative schematic of an example of a detection device for detecting analytes.

FIGS. 4A-4C depict illustrative schematics of examples of detection devices for detecting analytes.

FIG. 5 depicts an illustrative schematic of an example of an electronics system in a detection device for detecting analytes.

FIG. 6 depicts an illustrative schematic of an example of a sensor module in a detection device for detecting analytes.

FIGS. 7A-7D depict illustrative schematics of examples of sensor arrays in a detection device for detecting analytes.

FIG. 8 depicts an illustrative schematic of an example of a sensor chip for detecting analytes.

FIGS. 9A-9C depict illustrative schematics of examples of sensor chips for detecting analytes.

FIG. 10 depicts an illustrative schematic of an electrochemical sensor in a sensor chip, and analyte capture thereon, for detecting analytes.

FIG. 11 depicts an illustrative schematic of RTIL layers of an electrochemical sensor.

FIGS. 12A and 12B depict illustrative examples of an electrochemical sensor specificity in sensing an analyte.

FIGS. 13A and 13B depict illustrative schematics of examples of a base coupled to a sensor module in a detection device for detecting analytes.

FIGS. 14A and 14B depict assembled and exploded schematic views, respectively, of an example of a sensor module for detecting analytes.

FIG. 15 depicts an illustrative schematic of an example of a sensor module for detecting analytes wherein the sensor chips comprise gates.

FIG. 16 depicts an illustrative schematic of an example of a detection device for detecting analytes.

FIGS. 17A and 17B depict illustrative schematics of an example of a base coupled to a sensor module with a mouthpiece in a detection device for detecting analytes.

FIGS. 18A-18C depict assembled, assembled and translucent, and exploded views, respectively, of an example of a sensor module with a mouthpiece.

FIG. 19 depicts an illustrative schematic of airflow in an example of a nozzle in a sensor module.

FIGS. 20A and 20B depict top and bottom views, respectively, of an example of a circuit board with a sensor array and conductive traces.

FIG. 21 depicts an illustrative schematic of a method of detecting a target VOC.

FIGS. 22A and 22B depict illustrative schematics of detecting and/or tracking VOCs.

FIGS. 23A-23C depict illustrative data demonstrating detection of VOCs at two concentrations by a detection device.

FIG. 24 depicts illustrative data demonstrating calibration of sensors in a detection device for detecting COVID-19.

FIGS. 25A and 25B depict illustrative data demonstrating identification of healthy subjects and subjects that are presumptive positive for COVID-19 using two example electrochemical sensors in a detection device.

FIGS. 26A and 26B depict illustrative data demonstrating % change in sensor signal relative to an adjusted baseline characterization, for selected subjects referenced in FIGS. 25A and 25B.

FIGS. 27A and 27B depict illustrative data demonstrating % change in sensor signal relative to an adjusted baseline characterization, for a selected subject referenced in FIGS. 25A and 25B.

FIGS. 28A and 28B depict side and exploded schematic views, respectively, of an example of a mouthpiece in a detection device for detecting analytes.

FIG. 29A depicts an example variation of a detection system including a detection device and a mouthpiece coupleable to the detection device. FIG. 29B depicts a detailed view of the detection device shown in FIG. 29A.

FIG. 30 depicts an example variation of a detection system including a detection device and a sampling device coupleable to the detection device.

FIGS. 31A and 31B depict front and back surfaces, respectively, of an example variation of a sampling device for obtaining and storing a sample. FIG. 31C depicts a translucent perspective view of the sampling device shown in FIGS. 31A and 31B.

FIG. 32 depicts an illustrative schematic of a portion of a sampling device.

FIG. 33A depicts an illustrative schematic of a mouthpiece in an example variation of a sampling device. FIGS. 33B and 33C depict assembled and exploded views, respectively, of an inlet valve carrier assembly in the mouthpiece shown in FIG. 33A. FIGS. 33D and 33E depict assembled and exploded views, respectively, of an outlet filter carrier assembly in the mouthpiece shown in FIG. 33A.

FIG. 34A depicts an illustrative schematic of a compartment in an example variation of a sampling device. FIG. 34B depicts a partial cross-sectional view of the compartment shown in FIG. 34A.

FIG. 35A depicts an illustrative schematic of a connector and stopper assembly in an example variation of a sampling device. FIG. 35B depicts a partial perspective view of the connector stopper assembly shown in FIG. 35A. FIGS. 35C and 35D depict partial perspective and cross-sectional views of the connector shown in FIG. 35A. FIGS. 35E and 35F depict partial perspective and cross-sectional views of the stopper shown in FIG. 35A.

FIGS. 36A and 36B depict illustrative schematics of example variations of packaging for a sampling device.

FIGS. 37A and 37B depict illustrative schematics of an example variation of a sample extractor device.

FIGS. 38A-38D illustrate an example variation of method of using a sample extractor device.

FIG. 39A depicts an illustrative schematic of an example variation of a sheath. FIG. 39B depicts an illustrative schematic of a sheath in use with a detection device and mouthpiece to protect the detection device from contamination.

FIGS. 40A and 40B depict example variations of graphical user interfaces (GUIs) for a display used in connection with a detection device.

FIG. 41 depicts an illustrative schematic of an example variation of a detection device indicating a device status.

FIG. 42A depicts an example variation of a GUI for a display prompting entry of patient identification of a patient to be tested with a detection device.

FIG. 42B depicts an example variation of a GUI for a display indicating calibration status of a detection device.

FIGS. 43A-43C depict example variations of GUIs for a display providing instructions to a patient to provide a breath sample to a detection device.

FIG. 44 depicts an example variation of a GUI for a display providing guidance for a patient providing a breath sample to a detection device.

FIGS. 45A-45C depict example variations of GUIs for a display providing test results following analysis of a sample with a detection device.

### DETAILED DESCRIPTION

Non-limiting examples of various aspects and variations of the invention are described herein and illustrated in the accompanying drawings.

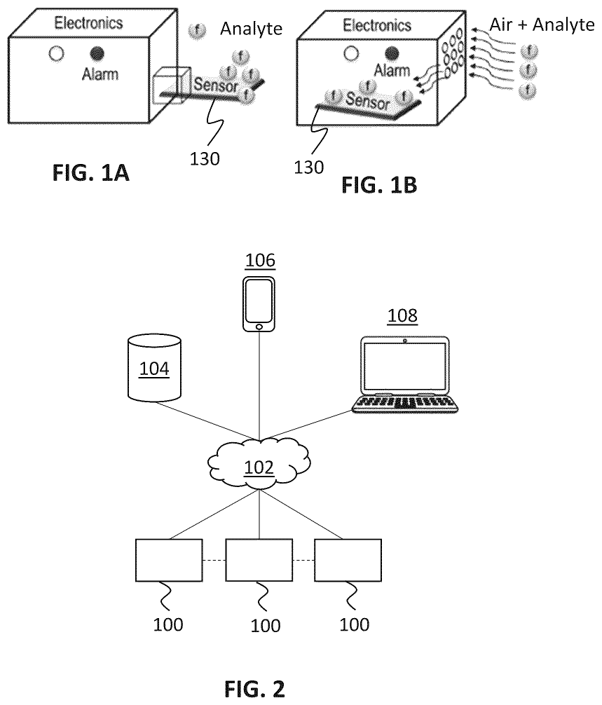

Described herein are variations of systems and methods for detecting one or more target analytes, including gas-phase chemicals. For example, systems and methods such as that described herein may be used to detect VOCs in a nearby or surrounding environment (e.g., for detection and/or tracking of threats). In some variations, such detection systems and methods may sense presence and/or distance of trace species (e.g., explosives, gunpowder, ammonium nitrate, opioids, biological agents, other trace VOC species, etc.). As another example, in some variations, systems and methods such as that described herein may be used to detect VOCs in breath of a user for diagnosis and/or tracking of medical conditions or other health state (e.g., COVID-19). As shown in the schematic of FIGS. 1A and 1B, a detection device 100 may provide an alarm or other suitable indication that a sensor module 130 detects an analyte (f). Detection may involve proximity sensing (e.g., a sensor module 130 may be placed in proximity to an analyte, such as that shown in the schematic of FIG. 1A) or distance sensing (e.g., a sensor module 130 may be placed at a location to detect an analyte in ambient environment, such as that shown in the schematic of FIG. 1B).

Furthermore, as shown in FIG. 2, detection systems described herein may include one or more detection devices 100 that may communicate with one or more remote devices (e.g., server 104, mobile device 106 executing a mobile application, other computing device 108, other detection device(s) 100) over a network 102 (e.g., cloud network, local network) to permit remote monitoring and/or other advantages of networked devices, as further described below.

In contrast to conventional detection devices, the detection systems and methods described herein have several advantages, including portability, ease of operation (e.g., do not require swabbing), the ability to provide continuous monitoring for threats or other conditions, and the ability to provide quantitative detection results. Additional beneficial features are described in more detail below.

### Systems for Detecting VOCs

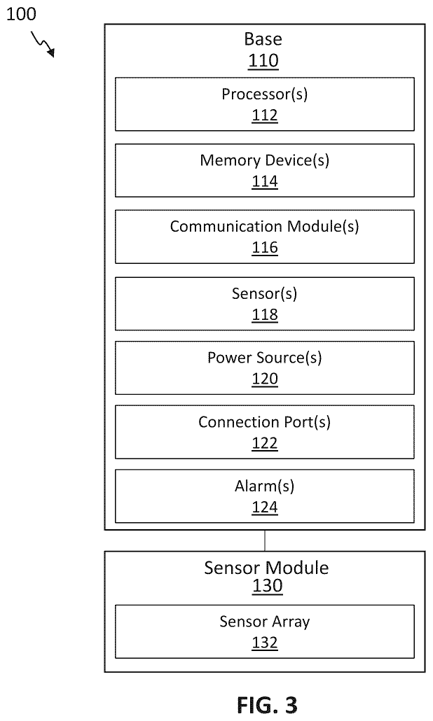

Generally, in some variations, a detection device for detecting a target analyte may include a base and a sensor module. For example, as shown in FIG. 3, a detection device 100 may include a base 110 and a sensor module 130. The sensor module may be coupled to the base and include at least one analyte sensor (e.g., electrochemical sensor). The analyte sensor may include an electrode and an ionic liquid (e.g., room-temperature ionic liquid (RTIL)) that is arranged on the electrode and specific to a target VOC, as further described below. Furthermore, in some variations, the sensor module may be removably coupled to the base, such that the sensor module may be interchangeable with other sensor modules (e.g., interchangeable with another sensor module after use, or interchangeable with other sensor modules that are specific to detecting different target VOC(s) for other detection applications). However, in some variations the sensor module 130 may be integrated with or permanently coupled to the base 110 (e.g., housed within the base, not removably coupled to the base).

### Base

As shown in FIG. 3, a detection device 100 may include a base 110 including various components, such as an electronics system including one or more processors 112 and memory device(s) 114 (e.g., for processing signals from the sensor module 130, and/or for handling other computational and processing actions of the overall detection device). The electronics system may further include one or more communication module(s) 116 configured to communicate with other devices, one or more additional sensors 118, one or more power source(s) 120, one or more connection port(s) 122 such as for access to data or for testing purposes, and/or one or more alarms 124 which may be configured to communicate information relating to detection of one or more target analytes.

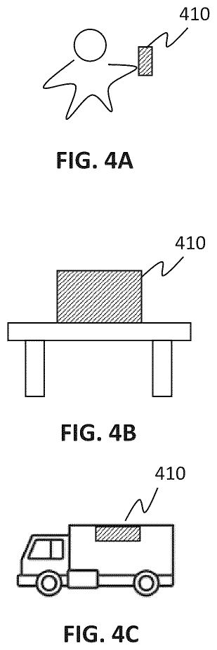

The base may have any suitable form factor and may be tailored for particular applications. For example, as shown in FIG. 4A, a base 410 may include a handheld housing which may be carried in a mobile manner by a person (e.g., as a mobile handset). In some variations, a handheld housing may include an ergonomic shape (e.g., curved, finger grooves, finger loops, etc.) to facilitate a more comfortable handheld grip. Additionally or alternatively, the handheld housing may include ribs, rubberized grip, and/or other suitable frictional features to help improve the ability of a user to grasp the handheld housing with ease and comfort. As another example, the base may be wearable by a person. For example, the base may include or be coupled to a strap, band, or helmet, shoulder mount, or other article of clothing and be worn on an arm, shoulder, hand, finger, wrist, leg, ankle, foot, torso, head, etc.

In some variations, a base 410 may include a standalone unit that may be placed on or otherwise mounted on a suitable surface, such as to rest on a table (FIG. 4B), counter, shelf, wheeled cart, or other surface, or mounted to a wall, ceiling, etc. For example, a base 410 may be placed in or on an automobile (FIG. 4C) such as a car or truck, an aircraft (e.g., plane, helicopter, etc.), a drone, a vessel, or other suitable item of ground, aerial, marine, or other vehicle of transportation. The base 410 may be placed in a cargo area or passenger area, or on an exterior surface of the vehicle. As another example, the base 410 may be optimized for a static, industrial setting (e.g., factory or other manufacturing facility, warehouse, etc.). However, the base may be configured for other suitable applications.

### Electronics System

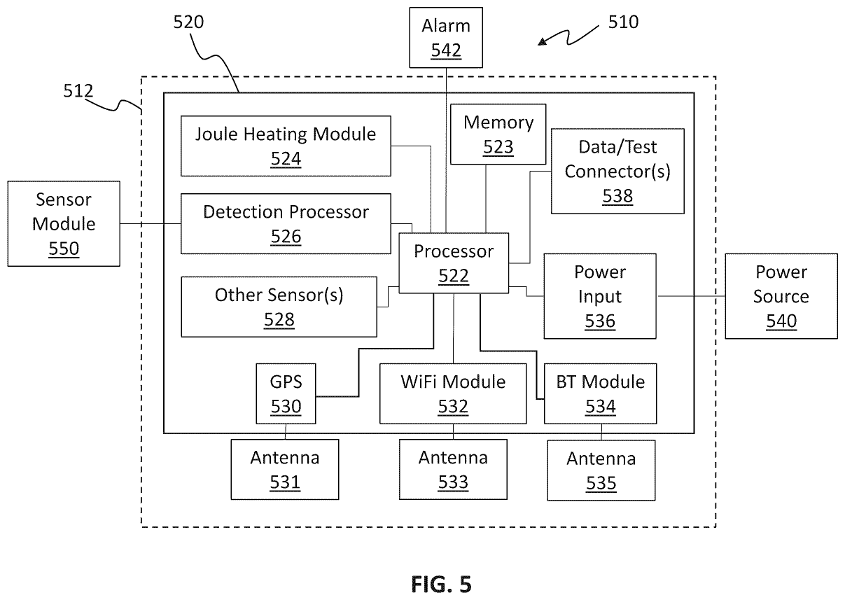

As described above, the base may include an electronics system. FIG. 5 depicts a schematic diagram of an example variation of an electronics system 510 for use in a base of a detection device. In some variations, the electronics system 510 may be used in a mobile handset-style detection device, for example. The electronics system 510 may include a circuit board 520 (e.g., motherboard) including various electrical components and/or connectors for peripheral components and/or cables. In some variations, the electronics system 510 may include multiple circuit boards 520, which may, for example, provide simultaneous or parallel functionality.

For example, the electronics system 510 may include at least one processor 526 configured to communicate with a sensor module 550 and perform computations specific for obtaining and/or interpreting electrical parameters of the analyte sensor(s) in the sensor module 550 in relation to detecting one or more target analytes (e.g., VOCs). For example, the processor 526 may be configured to perform electrochemical impedance sensing and/or chronoamperometry to measure and/or interpret impedance, current, and/or other suitable electrical parameters at the analyte sensor(s) in the sensor module 550. The processor 526 may, for example, be configured to detect and measure changes in one or more electrical parameters in a sensor signal from the analyte sensor(s), and correlate the change(s) to a detection of one or more target analytes. Such a detection processor 526 may, in some variations, be configured to detect current changes of 10 pA or less. For example, in some variations, such a detection processor 526 may include the EmState Pico Module available from PalmSens BV (The Netherlands).

The electronics system 510 may further include at least one processor 522 connected to other electrical components and configured for facilitating various other control and/or computational functions of the detection device. When executing instructions stored in one or more memory devices in the electronics system 510, the processor 522 may, for example, perform signal transmission and/or reception, sensor readings from additional sensors 528 as described in further detail below, time and/or frequency control of detections from the sensor module 550, encryption, diagnostics, health monitoring, and/or other suitable features. For example, the processor 522 may be configured to encrypt all signals being received and/or transmitted by the detection device, and/or configured to encrypt data stored on the device, using AES-256 encryption and/or any suitable encryption protocol or standard. For example, in some variations data may be encrypted using a combination of AES-128 encryption and an additional custom post-encryption algorithm that encrypts all data as it is being transmitted from the detection device. In these variations, 2 keys may be used to decrypt any transmissions, thereby increasing security of the transmitted information. Additional layers of encryption (requiring additional keys for decryption) may furthermore be applied to transmissions for increased security. In some variations, the processor 522 may include a microcontroller, a central processing unit (CPU), a field programmable gate array (FPGA), or any suitable processor chip(s). The processor 522 may have a clock speed of at least about 100 MHz which may, for example, help facilitate improved continuous monitoring and processing power. In some variations, a single processor may perform the combined features of processor 526 and processor 522.

In some variations, the base may include one or more memory devices 523 (e.g., flash storage) of any suitable capacity (e.g., at least 1 MB). A memory device 523 may, for example, store data prior to transmission by the electronics system 510. Additionally or alternatively, the base may include one or more ports for receiving a suitable memory device (e.g., SD card, miniSD, USB, etc.) which may be used to store data, provide software loading and/or diagnostics to and from the detection device, etc. A memory device 523 may, for example, include encryption capabilities using one or more techniques such as AES-256. In some variations, a memory device 523 may operate in parallel with a memory device of an auxiliary system (e.g., another computing device).

In some variations, the electronics system 510 of the base may include one or more sensors 528 that provide measurements of other parameters. For example, the electronics system 510 may include one or more sensors such as temperature sensors, humidity sensors, infrared sensors, ultrasonic sensors, radar sensors, gyroscopes, inertial measurement units (IMU), particulate sensors (e.g., PM10, PM2.5, PM1, etc.) and/or the like. Any one or more of the sensors 528 may be connected via conductive tracing, flex cables, and/or other suitable connection scheme. At least some of the sensors 528, such as temperature or humidity sensors, may provide measurements that may be useful for calibration of electrode sensors in the sensor module 550 and/or other sensors 528. Additionally or alternatively, at least some of the sensors 528 may provide sensor data useful for other monitoring and/or tracking applications, such as ambient temperature for environmental monitoring (e.g., in a refrigerated truck). In some variations, the electronics system 510 may be configured to receive data and/or transmit commands to sensors 528 at a frequency of at least 0.5 Hz (e.g., at least 0.5 Hz, at least 0.7 Hz, at least 1 Hz, etc.) or other suitable frequency. Furthermore, the electronics system 510 may be configured to receive data and/or transmit comments to the sensor module at a frequency of at least 10 Hz (e.g., at least 10 Hz, at least 15 Hz, etc.) or other suitable frequency. The electronics system 510 may further include suitable components for performing sensor signal processing (e.g., signal gain, signal filtering such as noise reduction for increasing signal-to-noise ratio, etc.), though additionally or alternatively one or more of the processors described above may perform digital signal processing. In some variations, the electronics system 510 may work in parallel with an auxiliary system (e.g., another computing device) to perform digital signal processing.

Furthermore, in some variations the electronics system may additionally or alternatively include a location sensor such as a GPS module 530 or GNSS with an associated antenna 531, which may enable location tracking of the detection device. While the sensors 528 and GPS module 530 are shown in FIG. 5 as part of the electronics system 510 in the base of the detection device, it should be understood that in some variations, one or more of the other sensors 528 and/or GPS module 530 may additionally or alternatively be located in the sensor module 550. In some variations, location tracking of the detection device may be performed through WiFi, and/or Bluetooth or similar communication between the electronics systems 510 of multiple detection devices through triangulation techniques.

In some variations, the electronics system 510 of the base may include at least one heating module 524 (e.g., Joule heating module). In some variations, the heating module 524 may include an electrical resistor heating element or other suitable element configured to produce heat upon input of a current. The heating module may, for example, function to regenerate a component of the sensor module 550. For example, at least a portion of the sensor module 550 may accumulate an undesirable substance (e.g., water moisture, non-targeted VOC, etc.) that may negatively impact the functioning of the sensor (e.g., fouling). The heating module 524 may remove such an undesirable substance by heating at least a portion of the sensor module to a suitable temperature that induces evaporation of the undesirable substance. The electronics system 510 may, for example, activate the heating module 524 as part of a calibration process (e.g., during manufacture and/or assembly of the detection device, upon detection device startup) and/or maintenance process (e.g., periodically, in response to detected environmental conditions such as humidity above a predetermined threshold, etc.).

The electronics system 510 may further include one or wireless communication modules such as a Bluetooth module 534 with associated antenna 535, and/or a wireless internet (WiFi) module 532 with associated antenna. Other wireless communication modules (e.g., radio, modules implementing cellular network technologies such as LTE, 2G, 3G, 4G, 5G, etc.) may additionally or alternatively be included. Furthermore, as described above, the electronics system 510 may include a location sensor such as GPS module 530 (or GNSS). In some variations, the Bluetooth antenna capability may be any suitable generation (e.g., Bluetooth 4.0 or later), the WiFi antenna capability may be configured to transmit in any suitable frequency (e.g., 2.4 GHz, 5 GHz, 24 GHz frequencies over a/b/c/g/n/ax spectrums, for example), and/or the GPS antenna capability may be configured to provide global positioning accuracy of at least 1 meter or less about every 10 seconds. Additionally or alternatively, the electronics system 510 may include at least one communications antenna of a custom signal frequency or frequencies. The communication modules may be configured to send and/or receive data in a wireless manner. Additionally or alternatively, the electronics system 510 may include any suitable communication module (e.g., wired or wireless communication modality). Through these signals, the detection device may communicate with one or more peripheral devices (e.g., server, mobile device such as a mobile phone or tablet, laptop or desktop computer, etc.). Such paired communication may, for example, enable communication of information between the detection device and the paired peripheral device (e.g., sensor data, user data, analysis data, sensor calibration data, software updates, etc.). Additionally or alternatively, in some variations, a detection device paired to a peripheral device (e.g., executing an application associated with the detection device) may utilize communication through its wireless communication module to locate itself in relation to the paired peripheral device. For example, if multiple detection devices are in close proximity to each other (e.g., in the same room), the peripheral device and/or any particular detection device paired to that device may indicate the location of the particular detection device and/or indicate which detection device is currently paired to the peripheral device. In some variations, multiple detection devices may be simultaneously paired to the same peripheral device, where the location and/or paired status of any of the paired detection devices may be indicated on the peripheral device and/or that detection device. In some variations, one or more detection devices may communicate via a wireless communication module to a peripheral device(s) that is near the detection device(s) such as in the same room. However, in some variations one or more detection devices may communicate via a wireless communication module to a peripheral device(s) that is distant from the detection device(s), such as not in the same room or even the same building. The latter scenario may be advantageous, for example, in instances where the detection device is configured to detect a target analyte that is associated with a contagious disease (e.g., exhaled metabolites in a sample of breath associated with a disease, as described in further detail herein), thereby keeping a user of the peripheral device (e.g., test administering personnel) safer from infection by a potentially contagious user operating a detection device and reduces the need for personal protective equipment for a user of the peripheral device.

In instances where multiple detection devices are in close proximity to each other, the paired communication between a peripheral device and one or more detection devices may enable helpful control over a particular detection device of interest. For example, in some variations, a peripheral device such as a mobile device may execute a mobile application having a “find me” operation that causes a detection device paired to that peripheral device to identify itself with a cue (e.g., through a user interface such as by illuminating an LED or other light element, displaying an indication on a display, sounding an audible indication). The cue from the detection device may be provided in a synchronous manner with a corresponding cue from the mobile application (e.g., notification message, vibration, etc.). Accordingly, a user of the peripheral device may interpret the cues from the detection device and/or the mobile application to identify which detection device among several nearby detection devices is paired to that peripheral device. In some variations, the mobile application may further be configured to change which nearby detection device that the peripheral device is currently paired to, if the user desires.

Additionally or alternatively, a detection device may communicate with one or more additional detection devices in a detection device network (e.g., mesh network such as a mesh network enabled by Bluetooth). Any one or more of such networked detection devices may, through such a network, be capable of locating itself in relation to the other networked detection devices, such as through Kalman filtering and triangulation. As another example, a detection device may perform a “health check” operation against other nearby detection devices to which the detection device is networked, where the detection device may check its sensor sensitivity level against that of other networked detection devices to help ensure that the networked detection devices are still operated in a suitable calibrated manner. Various methods of utilizing such a detection device network as part of a detection system are described in further detail below.

As described above with respect to FIG. 2, one or more detection devices may be configured to communicate with any suitable device, including a server or other suitable data storage device(s), over a network such as a cloud network. In some variations, data from a detection device (e.g., sensor data, user data, analysis data) may be communicated to one or more remote devices (e.g., cloud storage) via the wireless communication module of the detection device. Additionally or alternatively, data from one or more storage devices may be communicated from one or more remote devices to the detection device (e.g., sensor calibration data, software updates, etc.). Such data may be communicated substantially in real-time, such as if an internet connection or other wireless communication connection is available and active. In some variations, a detection device may store in its local memory (e.g., memory device 114) a volume of data and periodically or intermittently communicate batches of data to the one or more storage devices. Furthermore, if the detection device does not have an active wireless communication connection with the storage device(s), then in some variations the detection device may store data in its local memory until the wireless communication connection is available. For example, a predetermined number of test readings (e.g., 1000 test readings) may be locally stored until the detection device can synchronize over an available connection to a suitable remote or other storage device, thereby clearing local memory space to allow for more test readings to be stored.

In some variations, the electronics system 510 may include a power source or a connection port for accessing a power source. For example, as shown in FIG. 5, the electronics system 510 may include a power input 536 for coupling to a power source 540 (e.g., battery or other portable power source, or wired power source such as a wall outlet). In some variations in which the base is a mobile handheld unit, the base may include a portable power source such as a battery. In some variations in which the base is intended for vehicular transportation or in a static industrial setting, the base may draw power from the vehicle itself and/or include a portable power source. For example, the base may utilize a power source in the vehicle or industrial setting as a primary power source and utilize a portable power source in the base as a backup power source, or vice versa. Additionally or alternatively, the detection device may be powered through solar energy. For example, the power source may include or be coupled to at least one solar array. The solar array may be configured to charge the power source 540 or directly power the electronics system 510 of the detection device. In some variations, the solar array may provide a primary source of power, while in some variations the solar array may provide a supplemental source of power.

A portable power source 540 may be located, for example, within the base of the detection device (e.g., within a housing of the detection device). A portable power source may additionally or alternatively be located within the sensor module. In some variations, the electronics system 510 may be configured to receive power at a voltage of at least 3.3V, or any suitable voltage. The detection device may, for example, be rechargeable via any suitable connection (e.g., micro-USB and the like). In some variations, the electronics system may retain at least a lower threshold of battery reserve (e.g., 5%) at all times (e.g., before an automatic shut-off). This battery reserve may help enable the detection device perform minimum functions such as anti-tamper mechanisms, as described below.

Furthermore, as shown in FIG. 5, in some variations the electronics system 510 may include one or more data and/or test connector ports 538. Such connectors 538 may, for example, allow the electronics system 510 to be flashed with suitable software, tested, analyzed for diagnostics, and/or enable attachment to one or more peripheral device for extended capability (e.g., additional sensors, communication devices, display or other user interfaces, etc.). Other connectors (not pictured) may further enable connection between the base and the sensor module, including one or more electrically conductive contacts or cables for receiving and/or transmitting signals to the sensor module 550. In some variations, the electronics system 510 may be able to be flashed with suitable software, tested, and analyzed for diagnostics such as with a wireless communications module (e.g., Bluetooth module 534, WiFi module 532, etc.).

In some variations, the electronics system 510 may include at least one alarm system 542 configured to provide an alert in response to detection of a target analyte (e.g., target VOC). The alarm system 542 may additionally or alternatively provide an alert in response to a status of the detection device (e.g., low power, inoperability or fault detection, etc.). In some variations, the alert may be communicated on a user interface of the detection device such as that described below (e.g., display screen), and/or communicated via signaling such as visual signaling (e.g., illumination of LED lights) and/or audio signaling (e.g., through a speaker in a series of tones, beeps, etc.). Additionally or alternatively, the alert may be communicated to a peripheral or other remote device (e.g., mobile computing device, server, laptop or desktop computer, etc.), such as via a wireless communication module or other connector port, in order to indicate detection of a target analyte by the detection device and/or indicate a status of the detection device, and/or other suitable information.

### Other Base Features

As shown in FIG. 5, in some variations, the base may be configured to include protection against electromagnetic interference and/or thermal extremes. For example, the base may include shielding 512, which may, for example, provide precise EMI shielding from electromagnetic interference from component interaction within the base and/or peripheral interaction outside of the base. Additionally or alternatively, the base may include components (e.g., fins or other heat sinks, fans, etc.) that are configured to transfer excess heat from high energy components (e.g., processors, power source) away from thermally sensitive areas, such as toward an outer housing or other enclosure. The base may include one or more vents to promote cooling air circulation. The base may additionally or alternatively include features to substantially prevent heat return to the base.

Furthermore, the base may include structural reinforcements configured to brace against shock, pressure, and/or other structural requirements. As an illustrative example, the base may be configured to satisfy the structural and solar loading requirements under the MIL-STD-810 standard. As another example, the base may be sealed to withstand hydrostatic pressure of water up to a depth of at least about 100 feet. Additionally or alternatively, the base may be structurally robust to protect against environmental factors and/or user handling that may damage the detection device. In some variations, the base may include multiple housings or other enclosures to provide one or more of the above characteristics. For example, the base may include an endoskeleton chassis configured to brace against structural loading, as well as an exoskeleton enclosure including ridges and grooves to further protect against environmental factors and/or improve user handling (e.g., increase friction for better handling). The base may further include one or more mounts to attach the detection device to a suitable surface, such as through fasteners (e.g., magnets, adhesive, suction, etc.).

In some variations, the base may include one or more anti-tamper features. For example, the base may include a housing with one or more mechanical anti-tamper features and/or electronic-based anti-tamper features. Examples of mechanical anti-tamper features include mechanical interlocks, specialized fasteners requiring specialized or uncommon tools (e.g., Torx, star, or custom fasteners, etc.). In one example of an electronic-based anti-tamper feature, the processors within the electronics system of the base may include custom software whereby in order to disassemble the detection device, a permissive command must be sent to the detection device from an authorized peripheral device (e.g., executing companion custom software), where the permissive command contains an authentication key. Such an authentication key must be sent to the detection device in order to enable disassembly of the detector device (e.g., base and/or sensor module). If the authentication key is received, the detection device may be disassembled. If this authentication key is not received, an attempt to disassemble the detector device may cause a high-voltage current to be sent through critical circuitry to destroy or limit functionality of the device. Additionally or alternatively, an unauthorized attempt to disassemble the detection device may cause the detection device to automatically transmit an alert to a peripheral device (e.g., with the alarm system 542).

In some variations, the base may include a user interface. The user interface may, for example, include a display screen (e.g., LED display) configured to display information to a user. For example, as described above, the display screen may provide an alert from the alarm system, such as a signal indicating detection of a target analyte (e.g., target VOC) and/or a quantitative reading of amount of the detected target analyte. As other examples, the display screen may be configured to display icons providing status of network connectivity (e.g., Bluetooth, WiFi, cellular, etc.), information relating to power (e.g., on/off status, power level, recharging state, connectivity to external power source, etc.), system defaults (e.g., lack of connectivity to a sensor module), or any suitable status updates such as device status, sample status (e.g., confirming detection or receipt of a gas sample for analysis), detection status (e.g., detection of a target analyte, no detection of a target analyte, defective detection operation, analysis in progress, etc.), and/or other suitable information. In some variations, the user interface may additionally or alternatively include other forms of visual communication, such as LED light(s) where color, position, and/or sequence of lights may be translated into any of the above or other suitable information. For example, one or more LED lights and/or other suitable visual cues may be illuminated or otherwise activated to indicate any of the above or other suitable information (e.g., illumination of a red LED for detection of a target analyte, illumination of a green LED for no detection of target analyte). Additionally or alternatively, the user interface may include audio communication, such as a speaker configured to emit speech, tones, and/or other suitable audible cues to indicate any of the above information or other suitable information. Furthermore, a detection device may additionally or alternatively include suitable haptic (e.g., tactile) user interface features such as vibrations from a motor, etc. Furthermore, the base may include other suitable user interactive components, such as an identification module (e.g., fingerprint reader to record and/or verify identity of a user), a microphone, speaker, camera, etc.

### Sensor Module

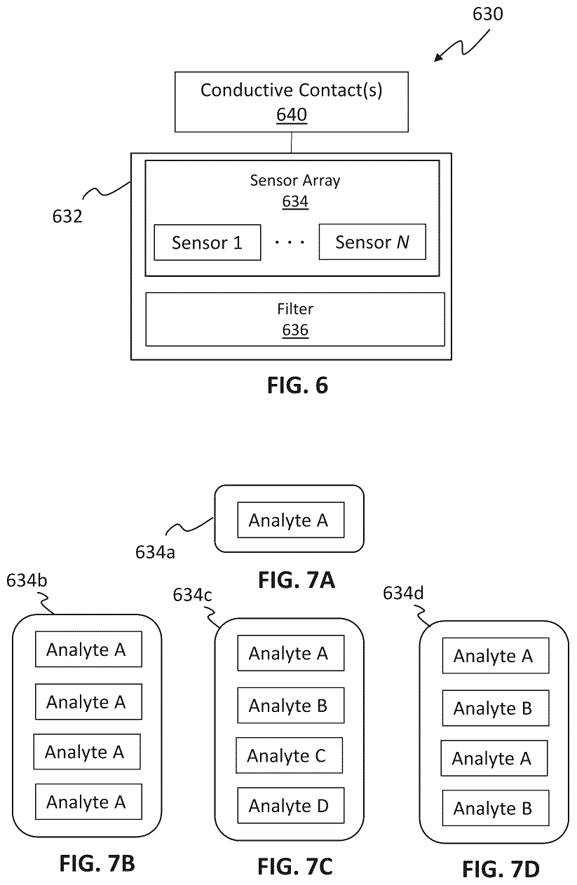

As shown in FIG. 3, a sensor module 130 may be configured to couple to the base 110. As shown in FIG. 6, a sensor module 630 may include a housing 632 and a sensor array 634 including one or more analyte sensors (also referred to herein as “sensor chip”). As described in further detail below, an analyte sensor may include an electrode and an ionic liquid (e.g., room-temperature ionic liquid (RTIL)) that is arranged over the electrode and is specific to one or more target VOCs. Accordingly, each analyte sensor may be specifically tailored for detecting a certain VOC or group of VOCs with sufficiently similar characteristics, as described in further detail below.

In some variations, the housing 632 may substantially enclose the sensor array 634. In some variations, the housing 632 may further function as a gate to help retain the RTIL or other ionic liquid over the electrodes of the sensor array. Example variations of housings for the sensor module are shown in FIGS. 14A-14B, FIG. 15, and FIGS. 17A-17B and are described in further detail below. However, the housing 632 may have any suitable size and/or shape for housing the sensor array 634.

The sensor module 130 may further include a filter 632 configured to filter out large air particulates, thereby reducing noisy substances that could interfere with functionality of the analyte sensors. In some variations, the filter may be positioned directly over and/or orthogonal to the electrode(s) of the sensor array, so as to filter in multiple directions relative to the electrode surface. In some variations, the filter 632 may form part of the housing 632. For example, FIGS. 14A-14B depict assembled and exploded views, respectively, of an example sensor module 1330 with a sensor module housing 1332. The sensor module housing 1332 may include a sensor module base 1334 coupled to a sensor module filter 1336, where the sensor module base 1334 and the sensor module filter 1336 forms an enclosure around the sensor array 1340. The filter 1336 may be formed from a sintered stainless steel material. In some variations, the filter may be formed at least in part from a sintered metal material (e.g., aluminum, steel (e.g., stainless steel), titanium, molybdenum, copper, etc. manufactured with sintering techniques). Suitable filter pore size for the filter 1336 may, for example, on the order of about 1 μm or larger. As another example, the filter may include a molecular sieve desiccant, such as an alkaline alumina silicate material, which may be formed into a suitable shape such as a spherical shape, with a pore size of about ten angstroms.

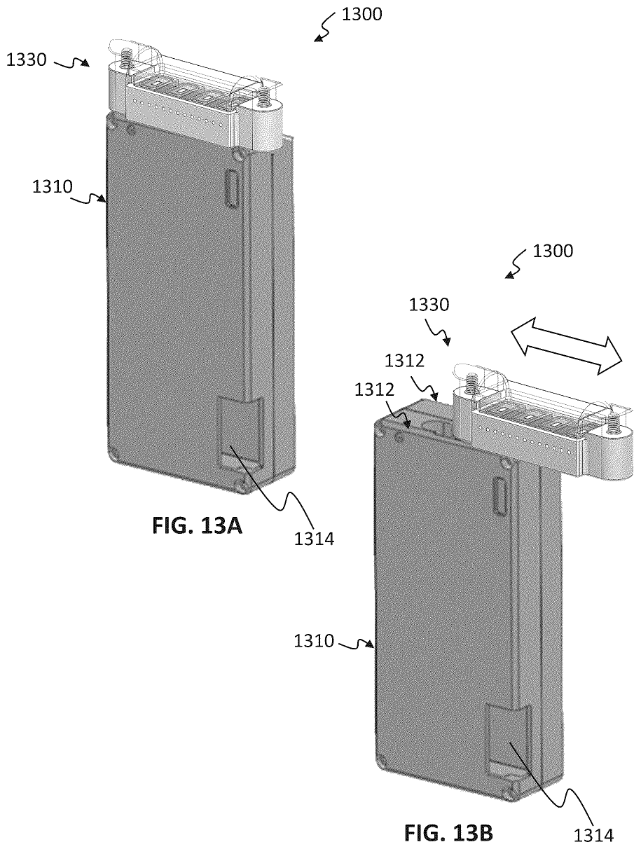

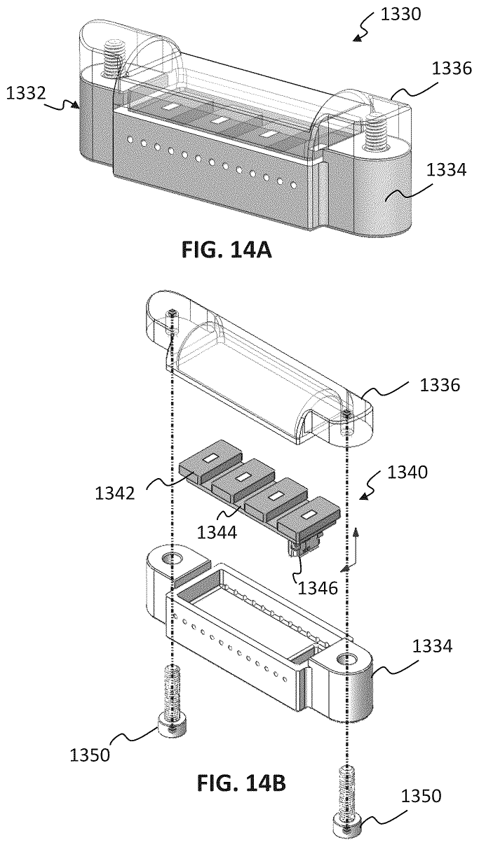

In some variations, the sensor module may removably couple to the base of the detection device, so as to enable swapping or interchanging of different sensor modules (e.g., to replace a used sensor module with an unused sensor module, to swap sensor modules that are specific to different target analytes, etc.). For example, in some variations, the base may include a set of one or more first engagement elements, and the sensor module (e.g., sensor module housing) may include a set of one or more second engagement elements. The first engagement elements and the second engagement elements may mechanically engage one another so as to couple the sensor module and the base together. Examples of engagement elements include slidingly engageable features (e.g., projecting features such as a tongues, splines, ribs, ridges, bumps, etc. that slidingly engage with recessed features such as grooves, etc.), snap fit features, fasteners that threadingly engage with threaded elements, and the like. For example, FIGS. 13A-13B depict an example variation of a mobile handheld detection device in which a sensor module 1330 is configured to slidingly engage and disengage in a lateral manner with a base 1310. Alternatively, sensor module 1330 and base 1310 may include snap fit features or other features that enable vertical separation between the sensor module 1330 and the base 1310 (e.g., along a longitudinal axis, or pivoting around a lateral axis with a hinged latch, etc.). While the sensor housing is primarily described and shown in the figures to be removably coupled from the base, it should be understood that in other variations, one or more of the sensor chips may additionally or alternatively be directly removable from the sensor housing for swapping. Furthermore, in some variations, the sensor module may be integrated or permanently coupled to the base of the detection device (e.g., housed within the base or sharing the same housing as the base, not removably coupled to the base).

The sensor module may include any suitable number of sensor chips. For example, for sake of illustration in FIG. 6, a sensor module 630 may include a sensor array 634 including N sensor chips. Furthermore, the sensor chips may be arranged in a single array, or in multiple arrays in any suitable manner. The sensor chips may be arranged in any suitable pattern or grouping, such as a linear array, a circular ring pattern, or the like. The sensor chips may be attached (e.g., mechanically and electrically) to a circuit board or suitable substrate that enables an electrically conductive path to the base, for communication of data, current, etc. to processor(s) within the base.

In some variations, a sensor array may include a single analyte sensor or sensor chip, which may be sufficient for detecting a target VOC using the detector device. For example, FIG. 7A depicts an illustrative schematic of a sensor array 634a including one sensor chip configured to detect a single analyte (Analyte A). However, in some variations a sensor array may include any suitable number of multiple analyte sensors or sensor chips, such two, three, four, five, six, seven, eight, nine, ten, or more analyte sensors. A plurality of analyte sensors may be utilized in various manners, described below with reference to FIGS. 7B and 7D.

In some variations, at least a portion of a plurality of analyte sensors may be specific to the same target analyte (e.g., the respective ionic layers of at least some analyte sensors may be specific to the same VOC, class of VOCs, or other analyte). For example, FIG. 7B depicts an illustrative schematic of a sensor array 634b including multiple sensor chips configured to detect the same target analyte (Analyte A). One advantage of this arrangement is redundancy. For example, in the event of a failure or malfunction of one of the sensor chips, the other similarly-configured sensor chips (that are specific to the same target analyte as the failed sensor chip) may still provide sufficient backup functionality and validation. As another example, redundant sensors may help reduce false positives, thereby increasing detection sensitivity. By way of illustration, if three of the sensor chips in FIG. 7B detect Analyte A and a fourth sensor chip does not, the detection device may conclude that the detection of Analyte A by the majority of the similarly-configured sensor chips is accurate and the detection device may respond accordingly (e.g., provide an alert indicating detection of Analyte A).

Another advantage of having multiple sensor chips configured to detect the same target analyte is that such an arrangement may enable tracking of direction and/or speed of the detected target analyte. By way of illustration, if all four sensor chips in FIG. 7B detect Analyte A but at different times, then given the known spacing and positions of the sensor chips and the timestamps of the sensor chips' detection of Analyte A, the detection device may calculate the direction and/or speed of travel of Analyte A. In other words, the arrangement of FIG. 7B enables the detection device to determine which direction the analyte is coming from, and how fast its travel is. In some variations, the detection device may further predict an anticipate travel vector for the detected analyte, by extrapolating to future direction and speed. Accordingly, predicting the past, current, and/or future travel of a detected analyte may, for example, provide useful information for monitoring and predicting threats associated with the detected analyte (e.g., which may provide advance warning before the detected analyte arrives as a particular location).

In some variations of sensor arrangements with multiple sensor chips, at least a portion of the analyte sensors may be specific to different analytes (e.g., the respective ionic layers of at least some analyte sensors may be specific to different VOCs, different classes of VOCs, or other analytes). For example, FIG. 7C depicts an illustrative schematic of a sensor array 634c including multiple sensor chips configured to detect different analytes (Analytes A-D). One advantage of this arrangement is that the sensor array 634c may be used to concurrently detect multiple substances in a single sensor array or in a single sensor module.

Furthermore, in some variations, one portion of a sensor array may include redundant sensor chips similar to that shown in FIG. 7B (i.e., multiple sensor chips that are specific to the same analyte) and another portion of a sensor array may include redundant sensor chips that target different analytes similar to that shown in FIG. 7C (i.e., multiple sensor chips that are specific to different analytes). For example, as shown in FIG. 7D, a sensor array 634d may include two sensor chips specific to Analyte A, and two sensor chips specific to Analyte B. Accordingly, the sensor array 634a has the advantages of redundancy and/or tracking of an analyte as described above with respect to FIG. 7B, as well as advantages of diverse concurrent detection of different analytes as described above with respect to FIG. 7C. It should be understood that the variations shown in FIGS. 7B-7D are only illustrative, and other similar variations may include sensor chips targeting any suitable number and combination of same or different analytes.

As shown in FIG. 6, the sensor module 630 may include one or more conductive contacts 640 that enable electrical communication of signals between the sensor module 630 and corresponding conductive contacts on the base of the detection device, when the sensor module 630 and the base are engaged. For example, conductive contacts 640 may be located on a surface of the sensor module housing 632 that interfaces with the base. In some variations, the conductive contacts may include contact pads (e.g., copper) with conductive traces that extend from the sensor chips in the sensor array 634. Each sensor chip may have a respective set (e.g., ground and signal) set of conductive traces. Furthermore, the conductive contacts 640 may include one or more conductive springs that are biased to ensure good electrical connection between the sensor chips and the base. Additionally or alternatively, the conductive contacts 640 of the sensor module may couple to corresponding contacts on the base of the detection device via cables (e.g., flex cables, etc.) including at least enough wires to transfer data and current, or connectors, etc.

### Analyte Sensor

In some variations, the sensor module may include one or more analyte sensors such as electrochemical sensors configured to perform electrochemical gas sensing. For example, as described above, the sensor module of the detection device may include at least one electrochemical sensor including at least one electrode and an ionically conducting medium such as an ionic liquid (e.g., RTIL). For example, the sensor module may include at least one reference electrode and/or at least one counter electrode, and at least one working electrode. Electrochemical gas sensing may be accomplished by amperometric sensing techniques (e.g., chronoamperometry), whereby a potential is applied to the electrode, and the resulting current is observed over time. The inclusion of the ionically conducting medium (transducer) aids in charge transfer, and allows conductive contact between a reference (and/or counter) electrode and a working electrode. Here, the sensor may utilize RTILs as selective transducers for chemical sensing of analytes using a chronoamperometric technique, for example. RTILs have properties that make them ideal for use as a transducer in an electrochemical sensor, such as high ionic conductivity, low volatility, wide electrochemical window, chemical stability, and high thermal stability. RTILs are advantageous over other electrolytes used in gas sensors because, for example, RTILs do not undergo decomposition at negative potentials and exhibit higher thermal stability.

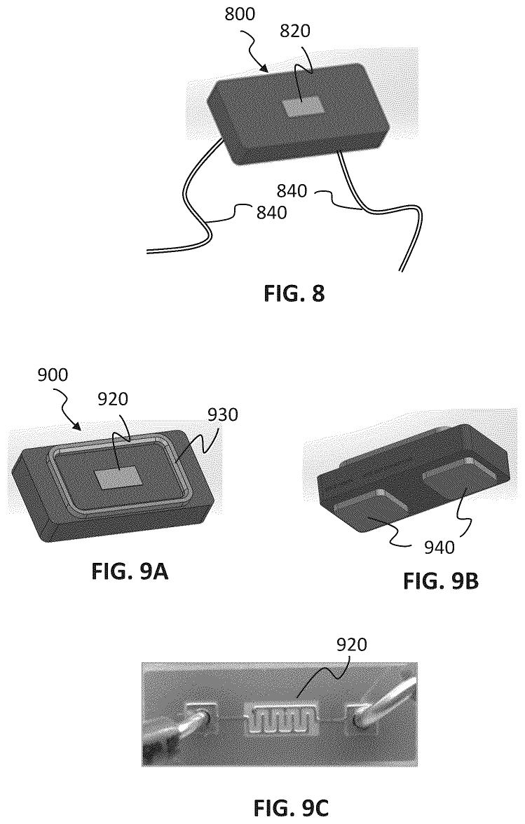

FIG. 8 depicts an example variation of an electrochemical sensor 800 or sensor chip including a nonconductive substrate. The electrochemical sensor 800 may include one or more electrodes 820 with an ionic liquid, such as a room temperature ionic liquid (RTIL), arranged over the electrodes. The ionic liquid (e.g., RTIL) may be specific to a target analyte of interest, as further described below. Furthermore, the sensor 800 may include one or more conductive contacts that are conductively coupled to the electrodes for carrying signals to and/or from the electrodes, such as via wiring 840. FIGS. 9A and 9B depict another example variation of an electrochemical sensor 900 or sensor chip similar to sensor 800, except that the sensor 900 further includes a gate 930 that may function to help contain a volume of RTIL arranged over the electrode. In some variations, the gate 930 forms a raised barrier (e.g., generally rectangular or other suitable shape) around the electrode, and may be deposited or otherwise coupled to the nonconductive substrate base of the sensor. In some variations, the gate may be made at least in part from a non-electrically conductive metal or composite material. Additionally, as shown in FIG. 9B, the sensor 900 may include contact pads as conductive contacts) to carry signals to and from the electrodes. Other conductive elements such as conductive traces, conductive springs, and/or suitable wiring, etc. may be conductively coupled to the conductive contacts of the electrochemical sensor.

The electrode(s) may be comprised of one or more suitable conductive materials such as a metal (e.g. gold) or a metal alloy. In some variations, the electrodes may include interdigitated electrodes (FIG. 9C), though the electrode may have any suitable shape (e.g., circular). The electrode material may, in some variations, be deposited onto the substrate using any suitable semiconductor manufacturing techniques.

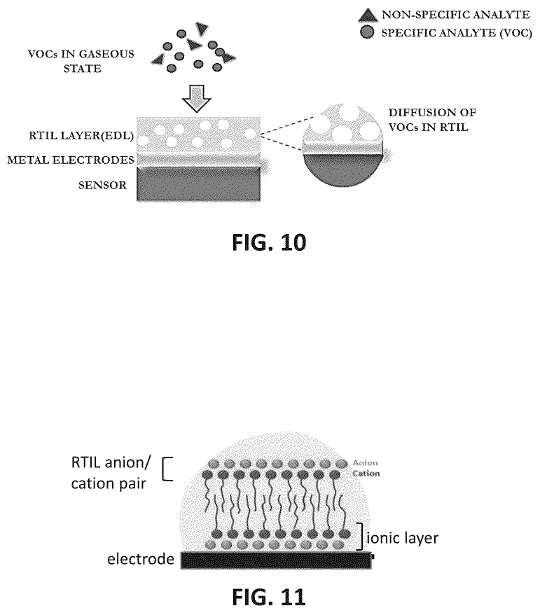

As shown in the illustrative schematic of FIG. 10, the RTIL may be deposited and arranged over the electrode(s). The RTIL may act as a transducer, selectively capturing VOCs, and allowing them to diffuse to the electrode interface where they are detected. In some variations, the volume of RTIL contained by the gate is between about 1 μL and 10 μL, between about 1 μL and 5 μL, about 1 μL, about 2 μL, about 3 μL, about 4 μL, or about 5 μL. In some variations, the thickness of the RTIL is between about 20 μm and about 150 μm, between about 20 μm and 100 μm, between about 20 μm and about 80 μm, between about 20 μm and about 50 μm, between about 50 μm and about 150 μm, between about 50 μm and about 100 μm, between about 50 μm and about 80 μm, between about 80 μm and about 150 μm, between about 80 μm and about 130 μm, between about 80 μm and about 100 μm, between about 100 μm and about 150 μm, or about 27 μm, about 54 μm, about 80 μm, about 108 μm, or about 135 μm. Generally, as the thickness of the RTIL increases, the number of interactions between the target analytes and RTIL increases, thereby improving response and sensitivity of the sensor. However, in some variations the thickness of the RTIL layer may be less than about 150 μm so as result in formation of a thin film instead of a larger droplet, as the larger droplet may result in a bulk effect that causes steric hinderance or does not facilitate VOC vapors to diffuse as readily toward the electrode sensor surface (thereby decreasing sensor response).

As shown in FIG. 11, in some variations, the RTIL may include a plurality of ionic layers due to the electrostatic interactions occurring between the cation and anion of the RTIL and the charged surface of the electrode. Each ionic layer includes a series of RTIL anion/cation pairs. In some variations, the RTIL may include at least 2 ionic layers. In some variations, the RTIL includes 3, 4, 5, 6, 7, 8, 9, 10, 15 or more than 15 ionic layers.

In one example, an electrochemical sensor may include a gold microelectrode onto which a thin layer of RTIL is dispensed. RTIL can be deposited on the electrode surface by manual deposition, by drop casting and spin coating technique, or other suitable deposition technique. Drop casting and spin coating the ionic liquid at a fixed angular speed may, for example, allow a more uniform thin layer to be formed and ensure robust sensor performance.

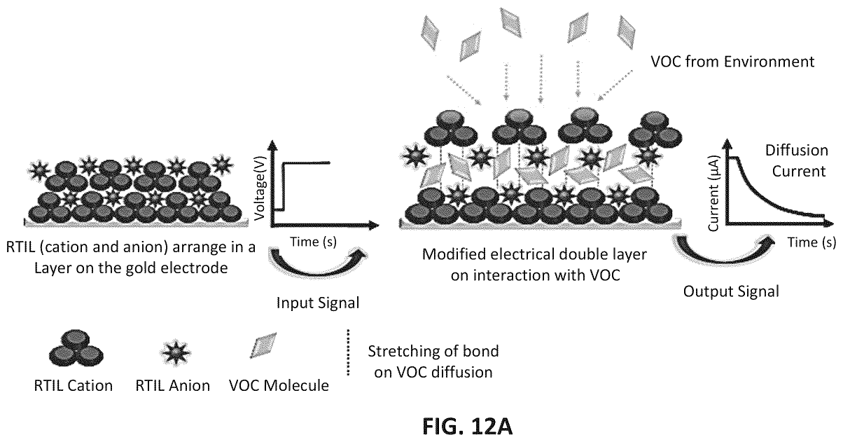

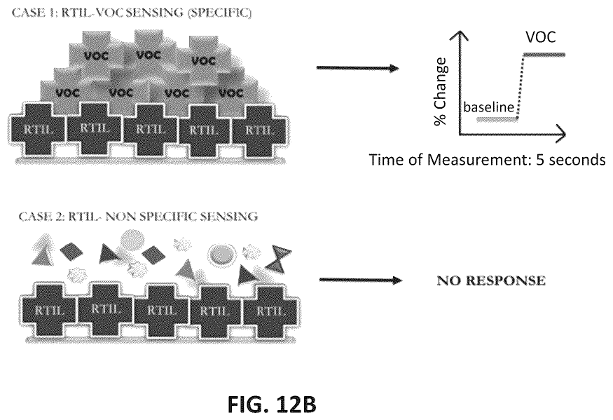

In a method of using the sensor to detect a target analyte, an input signal such as a DC voltage signal may be applied to the sensor. As shown in FIG. 12A, this input signal polarizes the cationic and anionic moieties of the RTIL, resulting in a stretching of the RTIL bonds. This stretching creates at least one nanoscale cavity that allows binding (capture) of target VOC molecules between ionic layers. In some variations, the size of the cavity corresponds to and depends upon the redox potential of the desired target VOC. In other words, application of an input signal (e.g., DC voltage, negative reduction potential) may cause formation of at least one cavity that is selective to the target VOC. For example, when a sufficient reduction potential is applied as an input signal, it may allow electron transfer to occur from the target VOC to the RTIL. This electron transfer can only occur if the applied potential matches the redox potential of the target VOC species. Interaction between the RTIL and VOC upon application of the reduction potential involves chemisorption of the molecules. These chemisorbed molecules may diffuse towards the electrode surface and cause change in current signal. The delta change in current is attributed to the number of VOC molecules diffused and is directly proportional to concentration of the target VOC.

However, the target VOC molecules are keyed to the cavity, and are able to bind within the cavity like pieces of a puzzle (FIG. 12B, Case 1). Diffused VOC molecules may be chemisorbed on the sensor surface. Chemical bond formation occurs between the ionic liquid species and the target analyte in a manner such that the molecules fit inside the RTIL cavities. This chemical bond formation is highly specific as it occurs between specific ionic species and functional groups present in the VOC. The captured target VOC is then able to diffuse toward the electrodes (e.g., working electrode), resulting in a change in current that is measurable in an output signal from the sensor. The change in current may be measured relative to a baseline current in an output sensor signal measured in the absence of the target VOC. For example, the change in current may be expressed as an absolute difference (new current relative to the baseline current, or as a ratio (new current divided by the baseline current, or vice versa). Because the induced cavity is specific to a particular target VOC, the sensor can detect the target VOC even amongst other gases have the same or similar concentration gradient as the target VOC at the surface of the electrode. This specificity is an advantage over other existing electrochemical gas sensors, such that those utilize a capacitance-based measure at the sensor surface and cannot distinguish between a target gas and competing gases having the same or similar concentration gradients.

As described above, the size and/or shape of the cavity corresponds to the redox potential of the desired target analyte. Accordingly, in some variations, a sensor containing a single RTIL may be used to detect a class of target analytes (e.g., VOCs) with the same redox potential. Furthermore, in some variations the input signal may be modulated (e.g., by tuning voltage amplitude) to vary the amount of stretching of RTIL bonds to match the redox potential of the target VOC of interest. In other words, in some variations a sensor with a single RTIL may in effect have a broad electrochemical window capable of detecting any of many possible target VOCs, by tuning the cavities to correspond to a particular target VOC.

Detection of a target analyte using an electrochemical sensor as described herein may occur quickly after receipt of a gas sample (e.g., a sufficient volume of gas for analysis). For example, in some variations, a determination of whether the target analyte is in the gas sample may occur within 5 minutes, within 4 minutes, within 3 minutes, within 2 minutes, within 1 minute, within 45 seconds, or within 30 seconds or less after receipt of the gas sample. As further described herein, an alert indicating the detection (or non-detection) of the target analyte may be provided via the detection device and/or a peripheral device or other suitable device that is communication with the detection device.

### Detection Device Examples

As described above, the detection device may have various suitable form factors. For example, FIGS. 13A-13B, 14A-14B, and 15 illustrate various parts of an example variation of a detection device 1300 including a handheld base unit 1310 and a sensor module 1330. As shown in FIGS. 13A and 13B, the sensor module 1330 may be removably coupled to the handheld base unit 1310. For example, FIG. 13A illustrates a configuration in which the sensor module 1330 is coupled to the handheld base unit 1310 through one or more engagement features (spines 1312) that engage corresponding engagement features on the sensor module 1330 (grooves, not pictured). The sensor module 1330 may be removably coupled to the base unit 1310 in a manner that enables ease of replacement. The sensor module 1330 may, for example be configured to be removable and/or disposable, similar to a replaceable cartridge. For example, in the event that the sensor array in the sensor module 1330 is faulty or degraded (e.g., decreased in accuracy), the sensor module 1330 may be swapped with another sensor module 1330. However, it should be understood that in some variations, the sensor module 1330 may be integrated with or permanently coupled to the base (e.g., housed within the base, sharing the same housing as the base, not removably coupled to the base, etc.).

The handheld base unit 1310 may include one or more connectors (e.g., protected within recess 1314) for permitting data communication to and from the handheld base unit 1310. Enclosed inside the handheld base unit 1310 may be an electronics system such as that described above (e.g., with reference to FIG. 5). The base unit 1310 may include a housing, such as housing shells coupled to one another through screws or other suitable fasteners, to enclose the electronics system. Although the base unit 1310 is shown in FIGS. 13A and 13B as generally rectangular prismatic, it should be understood that it may have any other suitable shape and/or other features. For example, the base unit 1310 may include a contoured, ergonomic shape (e.g., curved, finger grooves, finger loops, etc.) to facilitate a more comfortable handheld grip. Additionally or alternatively, the outer surface of the base unit 1310 may include ribs, rubberized grip, and/or other suitable frictional features to help improve the ability of a user to grasp the handheld base unit 1310 with ease and comfort. The handheld base unit 1310 may be formed at least in part of a suitable rigid or semi-rigid material (e.g., rigid plastic, metal, etc.) such as through injection molding, milling, 3D printing, or any suitable manufacturing process.

FIGS. 14A and 14B depict an assembled view and an exploded view, respectively, of a sensor module 1330 that may be coupled to the handheld base unit 1310 shown in FIGS. 13A and 13B. The sensor module 1330 includes a sensor module housing 1332 including a sensor module base 1334 (e.g., chassis) that may be coupled to a sensor module filter 1336 to substantially surround a sensor array 1340. The sensor module base 1334 and sensor module filter 1336 may be coupled together to house the sensor array 1340. The sensor module 1330 may be generally linear to accommodate a linear sensor array 1340 such as that shown in FIG. 14B, though may have other suitable shape. In some variations, the sensor module filter 1336 may include a half-cylindrical shape which may, for example, be configured to filter air approaching all the sensing surfaces of the electrodes of the sensor array 1340. The sensor module base 1334 may be made of a suitable rigid or semi-rigid material (e.g., plastic, metal, etc.) such as through injection molding, milling, 3D printing, or other suitable manufacturing process. In an exemplary variation, the sensor module filter 1336 may include a sintered stainless steel filter, or other suitable filter material.

As shown in FIG. 14B, the sensor array 1340 may be housed within the sensor module housing 1332. The sensor array 1340 may be seated on the sensor module base, for example, and may be further secured in the sensor module housing with fasteners (e.g., screw), epoxy, mechanical interfit features, etc. The sensor array 1340 shown in FIG. 14B includes a linear quad array of four sensor chips 1342 arranged (e.g., soldered) on a circuit board backplane 1344, though in other variations the sensor chips may be arranged in any suitable manner. The circuit board 1344 includes various conductive traces to communicate signals to and from the sensor chips 1342 to the base, such as via one or more conductive contacts in a connector 1346 (e.g., micro-USB connector).

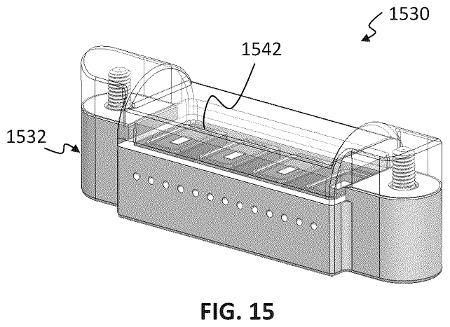

FIG. 15 depicts another example variation of a sensor module 1530 which may be removably coupled to a handheld base unit 1310. Like the sensor module 1330 depicted in FIGS. 14A and 14B, the sensor module 1530 includes a sensor module housing 1532 that houses a quad sensor array with one or more sensor chips 1542. However, in the variation shown in FIG. 15, each of the sensor chips 1542 additionally includes a gate configured to further retain RTIL on the electrode, as described above with respect to FIGS. 9A and 9B.

### Mouthpiece Variations

Exhaled breath can be used for non-invasive disease diagnosis. For example, respiratory diseases often alter metabolic pathways such as lipid peroxidation, and may upregulate the release of cytochrome P450 enzyme. The altered metabolic pathway releases VOCs in the breath, and these VOCs may be linked to specific respiratory pathways. Moreover, VOCs being released can be used for the diagnosis of diseases as their levels can be correlated to cellular metabolic pathways. Thus, VOCs in exhaled breath (e.g., present in parts per million to parts per billion levels) that arise from cellular in vivo metabolic activity may be used for diagnosis of diseases. For example, in a study performed by Violi, et al., their hypothesis is supported by Nox2 overactivation in Covid-19 patients with >40% increase compared to controls. (Violi, F. et al. (2020) “Nox2 activation in Covid-19”, Redox Biology, 36, p. 101655.) The study provides evidence that, compared to controls, Covid-19 patients display overactivation of Nox2, which is more marked in patients admitted to ICU. As another example, aliphatic hydrocarbons such as isopentane and heptane are also closely correlated to upper respiratory tract infections. (Jia, Z. et al. (2019) “Critical Review of Volatile Organic Compound Analysis in Breath and In Vitro Cell Culture for Detection of Lung Cancer”, Metabolites, 9(3).) Furthermore, compounds such as acetone have been found in headspace of cultured cells and correlated to respiratory infections. (Traxler, S. et al. (2019) “Volatile scents of influenza A and S. pyogenes (co-)infected cells”, Scientific Reports 9(1), p. 18894.) Detection of these and/or other biomarkers in an array-based manner may help improve the sensitivity and specificity of disease diagnosis.

In some variations, a detection device may be configured to operate with a mouthpiece for receiving an aerosolized sample from a user (e.g., breath). The detection device may, for example, be used to detect a health state (e.g., COVID-19) in a user based on detecting one or more target VOCs in exhaled breath from the user. Such target VOCs in exhaled may be used for diagnosis of diseases, as described herein.

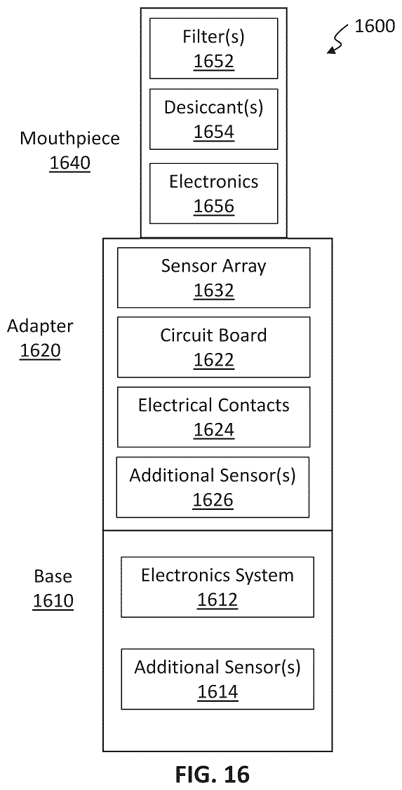

For example, as shown in the schematic of FIG. 16, a detection device 1600 may include a base 1610, an adapter 1620 coupled to the base, and a mouthpiece 1640 coupled to the adapter. The base 1610 may include an electronics system 1612, which may be similar to the electronics system described above with respect to FIG. 5. The adapter 1620 may function as a sensor module housing and include a sensor array 1632 with one or more electrochemical sensors, a circuit board 1622 providing a backplane for the sensor array 1632, and one or more electrical contacts 1624 for carrying signals to and/or from the sensor array 1632. The mouthpiece 1640 may include breath processing elements for preparing a volume of breath from the user before directing the breath toward the sensor array. Such breath processing elements may include, for example, one or more filters 1652 and/or one or more dehumidifying desiccants 1654. Additionally or alternatively, the mouthpiece 1640 may include features to help facilitate comfortable placement into a user's mouth for receiving a volume of breath, such as a curved edges, concave surfaces or other contours for lip placement, etc. Similar to the sensor modules described above, in some variations, the adapter 1620 may be removably coupled to the base or may alternatively be integrated with or permanently coupled to the base 1610.

In some variations, the mouthpiece 1640 may further include electronics 1656. For example, electronics 1656 may include an RFID chip (or other suitable communication chip) for use in near-field signal communication with the base 1610, adapter 1620, or other suitable computing device. The RFID chip may, for example, communicate information associated with the particular mouthpiece 1640, such as desiccant type, longevity or expiration date of the mouthpiece (e.g., due to desiccant drying out and expiring over time and/or use), or other suitable information. Additionally or alternatively, such information may be contained in a passive computer-readable code, such as a barcode or QR code, which may be readable with a separate scanner device and entered or otherwise communicated into the detection device (or other suitable computing device associated with the system), and/or scanned by an image sensor on the detection device itself (e.g., on the adapter 1620, base 1610, etc.).

Furthermore, the electronics 1656 may include one or more sensors (e.g., temperature, pressure, humidity, audio, etc.) for measuring one or more conditions in the mouthpiece and/or ambient environment, and/or one or more conditions of the breath or user. For example, the electronics 1656 may include a pressure sensor to measure airflow pressure received from the exhaled breath (which may, for example, be used to indicate whether a sufficient breath volume has been received, such as based on whether a sufficient pressure is measured over a threshold period of time). As another example, the electronics 1656 may include an audio sensor (e.g., MEMS microphone) that may be used to analyze miniscule audio patterns indicated in the user's breath that may be indicative of certain respiratory conditions. An RFID chip (or other suitable communication chip) may furthermore communicate any of the above-described sensor information to the base 1610, adapter 1620, or other suitable computing device.

Furthermore, in some variations, the base and/or sensor module may include one or more additional (e.g., auxiliary) sensors configured to measure one or more additional characteristics of a user. For example, as shown in FIG. 16 and described in further detail below, the base may include one or more additional sensors 1614 and/or the sensor module may include one or more additional sensors 1626 configured to provide other sensor measurements such as temperature, oxygen saturation, etc. of the patient. One or more additional sensors may additionally or alternatively measure ambient environmental properties such as temperature, humidity, etc. which may be used for sensor calibration purposes. In some variations, such additional sensors may additionally or alternatively be included on the mouthpiece 1640 (e.g., part of electronics 1656) as described above.

In some variations, the adapter 1620 and the mouthpiece 1640 may be part of a sensor module coupled to the base 1610. For example, similar to the sensor module 1330 described above, the sensor module including adapter 1620 and mouthpiece 1640 may be removably coupled to the base 1610 for swapping with another sensor module (e.g., between users to avoid cross-contamination). Additionally or alternatively, the mouthpiece 1630 may be removably coupled from the adapter 1620, such as for swapping or interchangeability (e.g., enable use of a differently-sized mouthpiece without replacing the entire sensor module). In some variations, the mouthpiece may be configured for single use or limited use (e.g., up to four or five times). For example, the mouthpiece may be configured for a single user to interact with the mouthpiece a single time or a limited number of times (e.g., for an assessment of a user which may require multiple samples of volumes of breath collected via the mouthpiece). In some variations, the mouthpiece may be disposable, such that the mouthpiece may be discarded after a user's breath is assessed with the detection device. Accordingly, the disposable mouthpiece may help maintain the sanitation of the detection device, and/or help prevent the airflow chamber of the sensor module from being exposed to contaminants for a prolonged period of time. However, it should be understood that in some variations, similar to other sensor modules described herein, the sensor module may be integrated with or permanently coupled to the base (e.g., housed within the base, sharing the same housing as the base, not removably coupled to the base, etc.).

In variations in which the mouthpiece 1630 may be removably coupled from the adapter 1620 (or otherwise from the rest of the detection device), the mouthpiece 1630 may include one or more keyed features (e.g., geometrical features such as a notch, unique or proprietary connection interface, etc.). Such keyed features may help prevent unauthorized use of other mouthpieces with the detection device (which may, for example, not include appropriate breath processing elements to help ensure an accurate breath assessment), and/or may function as an identification feature for identifying use of the mouthpiece for certain demographics (e.g., adult vs. pediatric). The mouthpiece 1630 may additionally or alternatively include visual and/or textural identification features, such as colored labels or raised ribs, etc.

Furthermore, in some variations, a detection device may omit the mouthpiece 1640, such that the sensor array 1632 may receive an aerosolized sample in a manner other than a user directly breathing into the mouthpiece. For example, the detection device 1600 may be configured to receive an aerosolized sample of a body fluid (saliva, nasal fluid, etc.) either directly or from a carrier such as a nasal swab. As another example, the detection device 1600 may be configured to receive an aerosolized sample from ambient air (e.g., if a user is standing near the detection device 1600). In such variations, the detection device 1600 may be used, for example, to detect a health state using aerosolized samples in addition or as an alternative to exhaled breath.

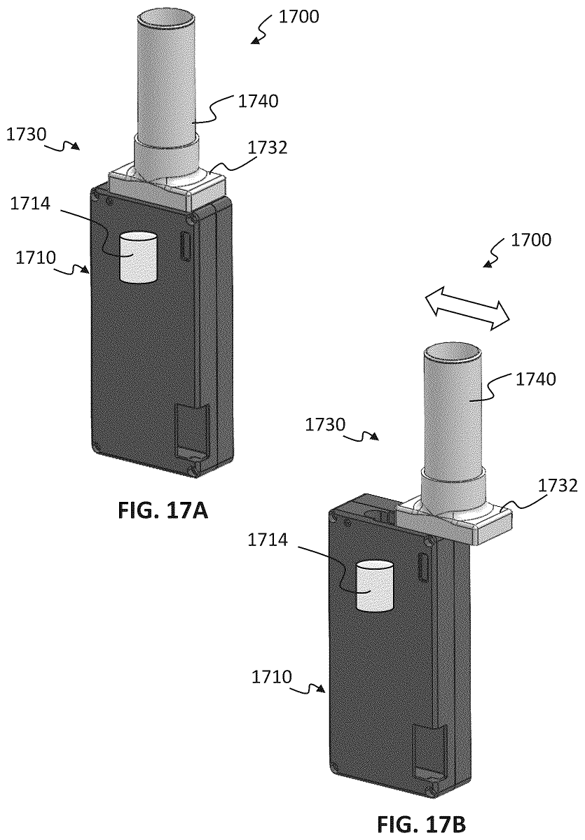

FIGS. 17A and 17B depict an example variation of a base 1710 and a sensor module 1730 including an adapter 1732 and mouthpiece 1740. The base may be a handheld base unit 1710 and the sensor module 1630 may be removably coupled to the base 1710, similar to the base and sensor module of detection device 1600 shown in FIGS. 13A-13B. At least a portion of the sensor module 1630 may be removable and/or disposable, similar to that described above. Alternatively, in some variations, the sensor module 1730 may be integrated with or permanently coupled to the base 1710.

In some variations, the base 1710 may further include one or more additional sensors 1714 configured to measure one or more characteristics of a user. For example, the sensor 1714 may include an infrared (IR) sensor for measuring temperature of the user. The IR sensor may, for example, be arranged on the base to measure temperature of a target with an optical axis that is generally parallel or aligned with the mouthpiece, so as to target the forehead of the user (or other suitable target) while the user's mouth is engaged with the mouthpiece 1740. In some variations, the optical axis of the IR sensor may be adjustable. For example, the IR sensor may be mounted in a pivotable, axially rotatable, and/or translatable mount to enable adjustment of the IR sensor optical axis relative to the base 1710. Additionally or alternatively, in some variations, the detection device (e.g., base and/or sensor module) may further include a targeting element (e.g., light beam or other source) configured to provide a visual indication of what location the IR sensor is targeting for measurement. The targeting element may be adjacent and generally parallel to the optical axis of the IR sensor, for example (e.g., the targeting element and the IR sensor may be co-located in the same mount or other structure on the base. In some variations, temperature information of the user gained from the IR sensor may be used to help characterize the medical condition of the user (e.g., detect or diagnose COVID-19, etc.).

As another example, the one or more additional sensors 1714 may include a pulse oximeter configured to measure oxygen saturation of the user. For example, the one or more additional sensors 1714 may include a PPG sensor mounted in a finger grip (or other suitable structure) so as to measure oxygen saturation of a user who is holding the base 1710. In some variations, oxygen saturation gained from the pulse oximeter may be used to help characterize the medical condition of the user (e.g., detect or diagnose COVID-19, etc.).

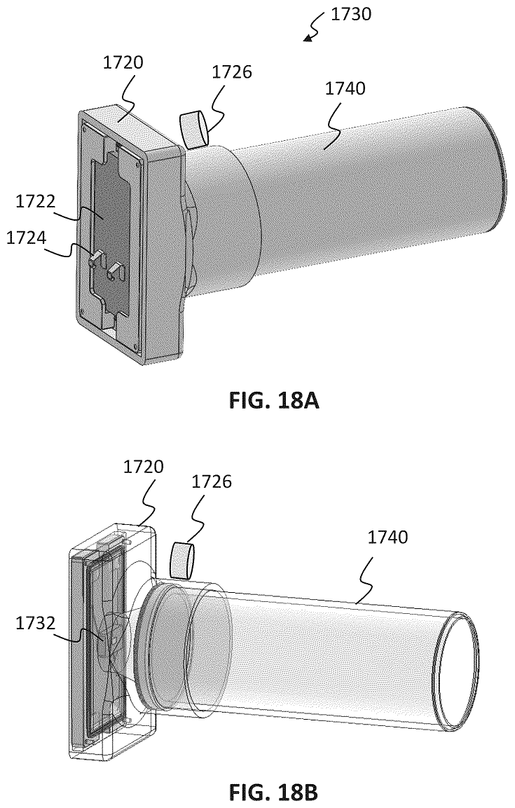

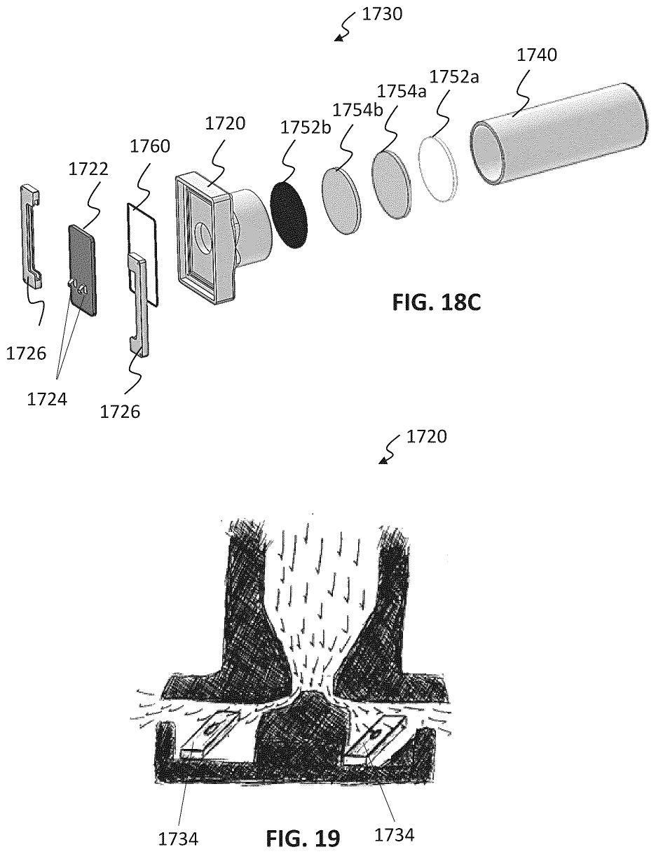

As shown in FIGS. 18A and 18B, the sensor module 1730 may include a mouthpiece 1740 and an adapter 1720. The mouthpiece 1740 may include a tube, and may be configured for easy replacement and disposal. For example, the tube may be a disposable plastic or cardboard mouthpiece. In use, a user may place his or her mouth on the tube and exhale, such that the tube directs the exhaled breath toward the adapter 1720 which includes at least one electrochemical sensor, as described below. Before reaching the electrochemical sensor(s), the exhaled breath may pass through one or more desiccants 1754 and/or filters 1752. At least one desiccant 1754 (or other dehumidifying element) and at least one filter 1752 may be arranged, for example, in series within the mouthpiece 1740, in any suitable order. In some variations, the filter 1752 may include a suitable filter material such as a metal, fabric, and/or composite material with filter pore size of at least 1 μm or larger. As another example, the filter may include a molecular sieve desiccant, such as an alkaline alumina silicate material, which may be formed into a suitable shape such as a spherical shape, with a pore size of about ten angstroms. The desiccant 1754 may include a suitable desiccant material such as a silica gel, dehumidifying clay, anhydrous calcium sulfate, and/or other hydrophilic materials. The geometry of a desiccant 1754 may, in some variations, resemble a rectangular prism, sphere, cylindrical prism, or pyramid. For example, cross-sectional geometry of desiccant 1754 may vary to optimize aerodynamic flow of breath (e.g., the desiccant may have a cross-section that is generally star-shaped, spiral, hexagonal, etc.). In some variations, a detection device may contain multiple filters 1752 and/or desiccants 1754 arranged in an array in the airflow path, such as in a linear, circular, or grid-like manner. For example, as shown in FIG. 18C, the mouthpiece 1740 may include two filters 1752a and 1752b and two desiccants 1754a and 1754b. A first filter 1752a may function as a pre-filter for filtering out larger particulates from the user's exhaled breath before it reaches the desiccants. The desiccants 1754a and 1754b may function to remove as much moisture (e.g., droplets) from the exhaled breath as possible. A second filter 1752b may be arranged after the desiccants and function as an airfoil to remove or reduce turbulent airflow entering the adapter 1720. However, any suitable number of filters and desiccants may be arranged in any suitable order within the mouthpiece.

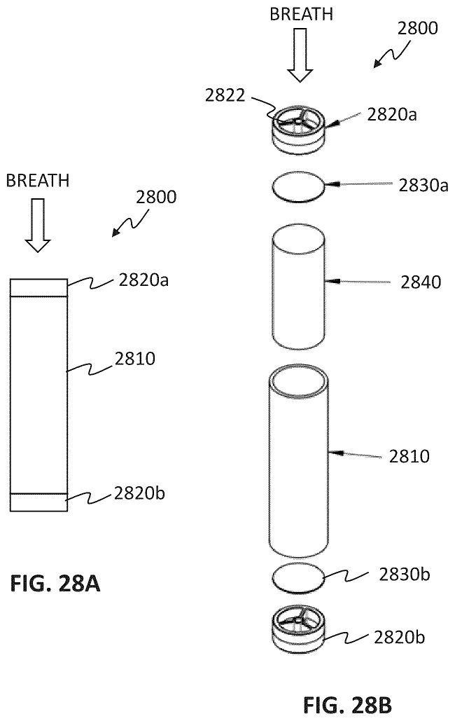

FIGS. 28A and 28B illustrate an example variation of a mouthpiece 2800 that may be used in a manner similar to that described above with respect to mouthpieces 1640 and 1740. The mouthpiece 2800 may, for example, be coupled (e.g., removably coupled) to an adapter such as adapter 1620 or 1720. For example, the mouthpiece 2800 may be coupled with mechanical interfit features (e.g., snap features, threads, etc.) and/or one or more fasteners. Like the mouthpiece 1740, the mouthpiece 2800 may be configured for easy replacement and disposal (e.g., include disposable plastic or cardboard). Alternatively, the mouthpiece 2800 may be permanently coupled or integrally formed with such an adapter.

As shown in FIG. 28A, the mouthpiece 2800 may include a tubular housing 2810 containing one or more breath processing elements. While the housing 2810 shown in FIGS. 28A and 28B is generally shaped as a circular tube, it should be understood that the housing may have other suitable shapes (e.g., elliptical cross-section, square cross-section, etc.). Furthermore, the housing may have non-uniform cross-sections. For example, in some variations, a mouth-receiving end of the housing 2810 may be generally flattened (e.g., elliptical, rectangular, etc.) which may be more comfortable for insertion into a user's mouth, while the housing 2810 may then take a rounder or other less flattened shape along its length as it approaches the adapter.

The housing 2810 may include a first housing end coupled to a first strainer disk 2820a, and a second housing end that is coupled to a second strainer disk 2820b. One or more of the strainer disks may, for example, be entirely or partially received (e.g., recessed into) the housing 2810 via mechanical interfit (e.g., snap fit) and/or one or more fasteners. In some variations, the strainer disks 2820a and 2820b may function to help direct breath from a user toward the sensor module of the detection device for assessment, extract large particles from breath, and/or help contain one or more breath processing elements within the housing. For example, one or both of the strainer disks may include one or more passageways (e.g., open rings) to receive breath directed into the mouthpiece by the user. A strainer material (e.g., mesh) may be arranged over such passageways to extract large particles from the user's breath, including large droplets (e.g., saliva, water, etc.) and/or other exhaled particles. Additionally, as shown in FIG. 28B, in some variations one or more both of the strainer disks may include ribs 2822 or other suitable containment features crossing the lumen of the housing 2810 that may help contain breath processing element(s) therein. Although FIG. 28B depicts three radial ribs 2822 radially arranged around the opening of the housing 2810, it should be understood that in other variations, the strainer disks may include any suitable number of radial ribs, radial ribs distributed unequally around the opening of the housing 2810, or in any suitable manner. Furthermore, one or more of the strainer disks may additionally or alternatively include other suitable containment features (e.g., chord-like lateral ribs, spiral ribs, fins, mesh, etc.).

As described above, the housing 2810 may include one or more breath processing elements, such as one or more filters and/or desiccants. For example, as shown in FIG. 28B, a desiccant 2810 may be arranged between a first filter 2830a and a second filter 2830b. The filters 2830a and 2830b and desiccant 2810 may, for example, include materials and/or have geometrical characteristics similar to that described above with respect to mouthpiece 1740. In use, breath from a user may pass through a first strainer disk 2820a as described above, and then through the first filter 2830a, which filters out smaller droplets and other exhaled particles that have not been extracted from the strainer disk 2820a. Breath may continue to pass through the desiccant 2810 which works to extract moisture from the airflow that has escaped the strainer disk 2820a and the filter 2830a. After passing through the desiccant 2810, the user's exhaled breath then travels through a second filter 2830b and a second strainer disk 2830b. Once the breath passes through the second strainer disk 2830b, the breath may proceed to the sensor module for assessment (e.g., of the user's health condition). Although FIG. 28B depicts an example arrangement of strainer disks, filters, and a single desiccant, it should be understood that other variations may include other suitable numbers of breath processing elements (e.g., two filters similar to filters 2830a and 2830b at each end, arranged in series) and/or other suitable combinations.

The adapter 1720 may include a housing for a sensor array 1732 including one or more electrochemical sensors. The sensor array 1732 may be arranged on a circuit board 1722 placed in the adapter 1720, as shown in FIGS. 18A and 18B. For example, as shown in FIG. 18C, a circuit board 1722 may be received in a recess of adapter 1720, such as with one or more settings 1726 (e.g., brackets) to help place and/or secure the circuit board 1722 within the adapter 1720. Furthermore, in some variations one or more sealing elements 1760 (e.g., O-ring) may be arranged in the adapter 1720 so as to help seal air flow within the adapter 1720 and retain an aerated sample within a chamber or suitable air path, such that the aerated sample is guided over the one or more electrochemical sensors in the adapter 1720.

In some variations, the adapter 1720 may include a nozzle configured to laminarize airflow over the sensor array. For example, as shown in FIG. 19, the adapter 1720 may include a bifurcation funnel to channel the air into two paths (or additional paths) of laminar flow, each path passing over at least one respective electrochemical sensor 1734. In other words, the adapter 1720 may cause laminar flow along the detection faces of the sensors. After passing over the sensors 1734, the airflow in the adapter 1720 may exit out vents or other openings in the adapter 1720. Although the adapter shown in FIG. 19 includes two sensors on opposite sides, it should be understood that the sensor array in the adapter may include any suitable number of sensors (e.g., one, three, four, five, or more) arranged in any suitable pattern (e.g., equally divided on opposite sides to receive bifurcated airflow streams, radially arranged, linearly arranged, etc.). Accordingly, the nozzle may divide the airflow in an appropriate number of channels depending on the number of sensors. Furthermore, multiple sensors in the same device may be specific for the same target analyte, or at least some sensors in the same device may be specific for different target analytes as described elsewhere herein. In some variations, the adapter 1720 may include one or more mechanical fins (and/or members or projections having fin-like geometry) to direct the airflow in one or more specific directions to promote laminar flow onto the sensor(s). These mechanical fins may additionally or alternatively direct airflow into an array of filters 1752 and/or desiccants 1754 (e.g., described above) in order to filter and dehumidify the air passing over the sensors 1734. In some variations, these fins may be able to rotate, tilt, and/or translate in specific directions in order to direct the airflow into a desired direction. The rotation and translation of these fins may be reactive to certain airflow pressures and/or electronically adjustable through the electronics system 510.

In some variations, the adapter 1720 and/or base of the detection device may include one or more user interface elements to provide feedback to a user regarding how much breath volume has been provided to the detection device and/or to provide guidance as to whether to provide a supplemental breath sample. For example, the adapter 1720 and/or base of the device may include audio and/or visual elements to communicate such information (e.g., LED lights, screen, speaker, etc.). As another example, the adapter 1720 and/or base may include tactile feedback elements (e.g., vibration motors) to communicate feedback information.

Furthermore, in some variations, the sensor module 1730 may include one or more additional sensors. For example, as shown in FIGS. 18A and 18B, one or more additional sensors 1726 may be arranged on the mouthpiece 1740 (though it should be understood that additionally or alternatively, such one or more additional sensors 1726 may be arranged on the adapter 1720). In some variations, the one or more additional sensors 1726 may include an IR sensor configured to measure temperature of a user. The function, alignment, and/or adjustability of such an IR sensor may, for example, be similar to that described above with respect to sensors 1714 shown in FIGS. 17A and 17B. Furthermore, the detection device (e.g., base and/or sensor module) may include a targeting element similar to that described above in connection with sensors 1714 to help indicate the location of temperature measurement. As another example, the one or more additional sensors 1726 may additionally or alternatively include a pulse oximeter configured to measure oxygen saturation, similar to that described above with respect to sensors 1714.

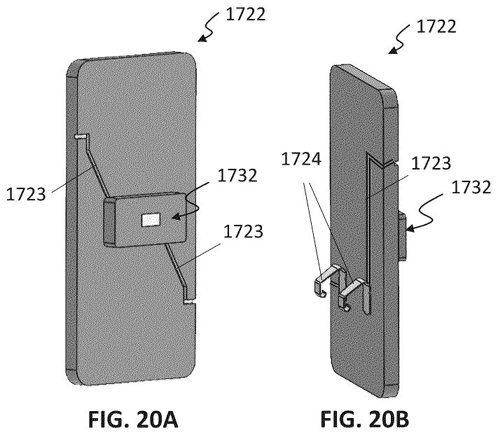

The sensors 1734 may include sensor chips including an electrode and an ionic liquid (e.g., RTIL) arranged over the electrode. As shown in FIGS. 20A and 20B, the sensor array 1732 may be soldered onto the circuit board 1722, and the circuit board 1722 may further include conductive traces 1723 (e.g., copper or other suitable conductive material) to carry signals to and from the sensor array and/or one or more additional sensors 1726. The conductive traces may extend to electrical contacts 1724 which are configured to conductively couple to the base unit for sensor signal processing. For example, as shown in FIGS. 20A and 20B, the conductive traces may wrap from sensor array side (FIG. 20A) around the circuit board 1722 to a base side (FIG. 20B), and conductively couple to the electrical contacts 1724 on the base side of the circuit board. In some variations, the electrical contacts 1724 may be springs (as shown in FIG. 20B) made of a conductive material, where the springs are outwardly biased toward the base, so as to urge and help ensure consistent electrical contact with corresponding electrical contacts on the base. Accordingly, sensor signals from the sensor array 1732 may be carried to the base for processing via the conductive traces 1723 and electrical contacts 1724.

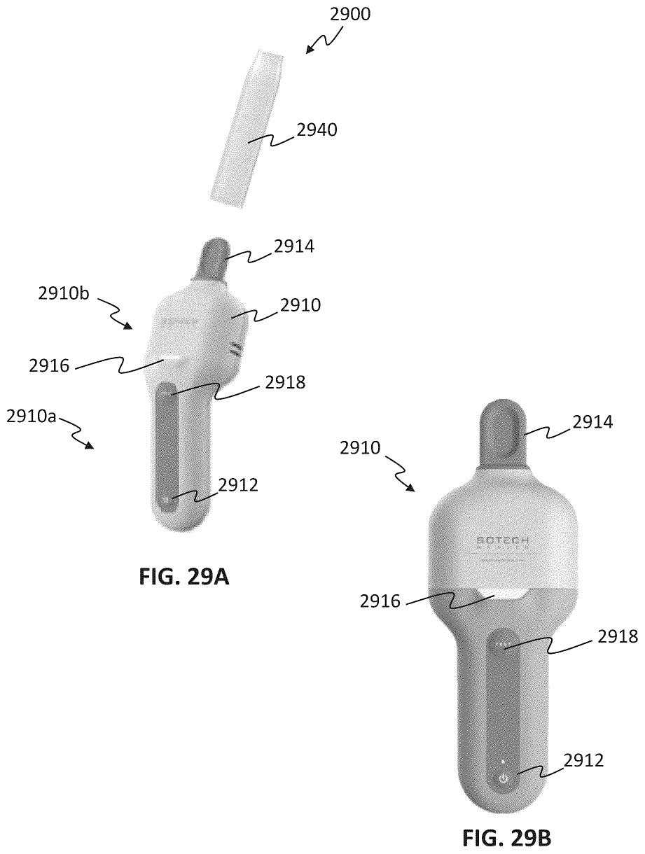

FIG. 29A depicts an example variation of a detection system 2900. Detection system 2900 may include a detection device with a handheld housing 2910 including a base and/or sensor module with features similar to that described above, and a mouthpiece 2940 with features similar to that described above. As shown in FIG. 29A, the handheld housing 2910 may include a handle or gripping portion, and may include user interface features such as a power button 2912 that is accessible by a user handling the detection device to turn on and off the detection device, a “test” button 2918 to initiate a test (e.g., initiate a sampling procedure) of a sample for analysis, and/or an indicator 2916 (e.g., illumination element, such as an LED) configured to indicate a status of the detection device and/or results of sample analysis. Although many of these user interface features are shown in FIGS. 29A and 29B on a handle portion of the detection device, the user interface features may be on any suitable portion of the detection device. Furthermore, as described above, the detection device may additionally or alternatively include other suitable user interface features (e.g., speaker, display, and/or actuator to provide audio, visual, and/or tactile feedback to a user).

As shown in FIGS. 29A and 29B, a proximal portion 2910a of the detection device may be shaped as an elongated member, but may alternatively have any suitable shape (e.g., bulbous or contoured). A distal portion 2910b of the housing 2910 may, in some variations, house at least a portion of an electronics system, sensor module, etc., though in some variations at least a portion of the electronics system and/or sensor module may be housed within the proximal portion 2910a of the housing 2910. In some variations, the proximal portion 2910a may include textural features (e.g., ribs, finger contours, frictional materials such as silicone, etc.) to improve grip and/or ergonomic handling of the housing 2910. Furthermore, the proximal portion 2910a may be angled relative to the distal portion 2910b (e.g., between about 100 degrees and about 170 degrees), which may, for example, improve a user's access to the mouthpiece when the user is holding the housing 2910. The housing 2910 may include an adapter or other suitable connecting interface configured to engage with the mouthpiece 2940. For example, the adapter may be inserted into a cavity of the mouthpiece 2940 and engage the mouthpiece in a snap-fit manner, or via any other suitable connection interface. Accordingly, the mouthpiece 2940 may couple to (e.g., be in fluidic communication with) the sensor module in the housing 2910 such that the mouthpiece directs a volume of breath to the sensor module. In some variations, the detection system 2900 may include a removable plug 2914 configured to engage with the connecting interface, such as in the absence of the mouthpiece 2940 (e.g., when the detection system is not in use, such as during transport, storage, between users, etc.).

In variations such as those described above, following processing of the sensor signals, the detection device may provide one or more alerts indicating detection of a target analyte if the device concludes that the sensor signals indicate presence of the target analyte. For example, the detection device may provide an alert through a user interface on the detection device (e.g., blinking LED light, audible signal, tactile signal such as vibration, etc.), a user interface of a peripheral computing device (e.g., a mobile application executed on the computing device such as a mobile phone or tablet), or to a server, etc. such as that described above. The user interface may additionally or alternatively provide other suitable information such as an indication of device status and/or sampling status (e.g., readiness to receive a breath sample, sufficient breath sample obtained, error occurrence, etc.) via visual, audible, tactile, and/or other suitable cues. For example, the detection device may illuminate a light element (e.g., LED), emit an audible cue, and/or vibrate in order indicate to a user that the detection device is ready and awaiting a breath sample, that the detection device has received a sufficient volume of a breath sample, that one or more target analytes have been detected in the breath sample, that one or more target analytes have not been detected in the breath sample, and/or that an error has occurred (e.g., mouthpiece is not correctly coupled to the sensor module). It should be understood that any of the above information may additionally or alternatively be communicated to another device in communication with the detection device (e.g., paired peripheral device such as a mobile computing device executing a mobile application).

For sake of illustration, operation of a detection device with a mouthpiece is described below with reference to the example variation of the detection device shown in FIGS. 29A and 29B (though it should be understood that other variations of detection devices may be operated in a similar manner). In some variations, the detection device may selectively be used without or with a paired connection to a mobile application on a computing device. In instances in which the detection device is used without a paired connection to a mobile application on a computing device, information (e.g., instructions, device or sampling status, etc.) may be communicated via an indicator 2916 that may be controllable to illuminate with different colors, spatial patterns, and/or temporal patterns. For example, after the detection device is powered on (e.g., by activating the power button 2912), the indicator 2916 may be illuminated with a ready signal (e.g., white illumination) to communicate that the detection device is ready to test. A user may press the “test” button 2918 to initiate a sampling procedure, and the indicator 2916 may then change appearance to communicate that the detection device is preparing itself to receive a sample. In some variations, the indicator 2916 may further change appearance to communicate a countdown procedure prior to expecting receipt of a sample. For example, the indicator 2916 may illuminate a color sequence (e.g., red, yellow, then green illumination) to indicate a countdown to when the user should begin exhaling into the mouthpiece 2940. The user may continue exhaling into the mouthpiece 2940 until the indicator 2916 changes appearance again (e.g., sustained red illumination) to communicate that a sufficient sample has been received and the user can stop exhaling. The detection device may then analyze the gas sample that has passed through the mouthpiece to the sensor module in the detection device, and the indicator 2916 may communicate results of the analysis. For example, the indicator 2916 may illuminate with a first predetermined color and/or timing (e.g., blinking red illumination) to indicate a positive screening where the target analyte was detected in the exhaled breath sample. The indicator 2916 may illuminate with a second predetermined color and/or timing (e.g., blinking green illumination) to indicate a negative screening where the target analyte was not detected in the exhaled breath sample. Additionally or alternatively, the indicator 2916 may illuminate with a third predetermined color and/or timing (e.g., solid blue illumination) to indicate that an error occurred and/or that a retest is required to obtain a test result. Once the results have been obtained, the results may be saved and/or communicated to one or more storage devices, and the mouthpiece may be disposed of (e.g., as biohazard waste). The detection device may then be sanitized (e.g., with alcohol wipes) prior to use with another user and/or prior to powering off the detection device. In instances in which the detection device is used with a paired connection to a mobile application on a computing device, some or all of the information communicated via the indicator 2916 as described above may additionally or alternatively be communicated via a display or other user interface on the computing device.

Detection System with Sampling Device

In some variations, a detection system may include a sensor module and a sampling device coupleable to the sensor module, where the sampling device may be sealable and configured to store a volume of a sample (e.g., gas) to be analyzed by the sensor module. For example, the sensor module may include at least one electrochemical sensor that is specific to a target VOC, such as that described above. In some variations, the sampling device may be configured to separately capture and store a sample for analysis (e.g., a volume of breath), and then coupled to the sensor module of a detection device for analysis.

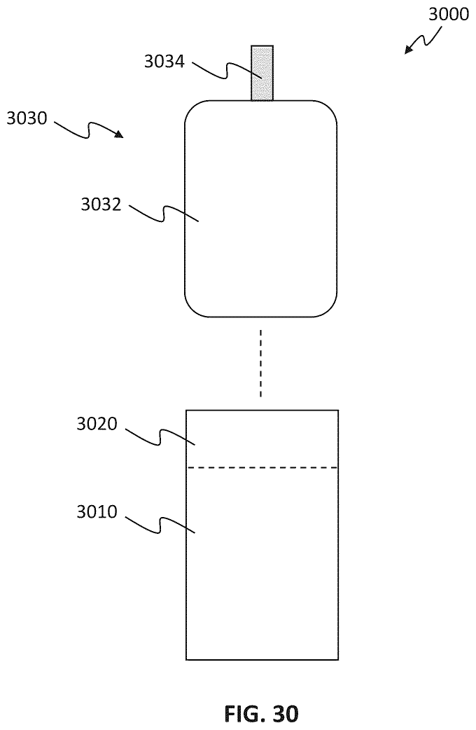

For example, as shown in FIG. 30, a detection system 3000 may include a sensor module 3020 and a sampling device 3030. In some variations, the sensor module 3020 may be coupled to or incorporated into a base 3010 (which may be handheld, a standalone device such as kiosk, etc.). The sampling device 3030 may include a compartment configured to store a volume of a sample such as breath or another volume of one or more gases. In some variations, the sampling device 3030 may include a mouthpiece 3034 (which may be similar to mouthpieces such as that described above) for use in transferring a volume of breath from a subject into the compartment. The sampling device 3030 may capture and store the sample while the sampling device 3030 is decoupled from the sensor module 3020 and/or rest of a detection device. In an example use scenario, multiple sampling devices 3030 may be provided to a plurality of subjects, each of whom can exhale into the mouthpiece of a respective sampling device 3030 that stores the subject's breath. Each sampling device 3030 may be labeled or otherwise identified as associated with its respective subject, so as to correlate each subject with his or her sample. At an appropriate time, these sampling devices 3030 may then be coupled to one or more detection devices including a sensor module, and each sample may be analyzed by the sensor module to identify whether the target VOC is present in the sample. These sampling devices 3030, along with their stored samples, may be transported and/or stored as necessary prior to being coupled to a detection device. In some variations, the sampling devices 3030 may be single-use consumables.

In some variations, a detection device may be used to process multiple samples in a set of sampling devices 3030. Accordingly, the detection system with sampling devices 3030 may be used to process the samples from multiple users in an easy-to-use, efficient manner (e.g., for mass testing applications), and reduce the number of separate detection devices that need to be simultaneously accessible in order to process samples from a group of subjects.

### Sampling Device

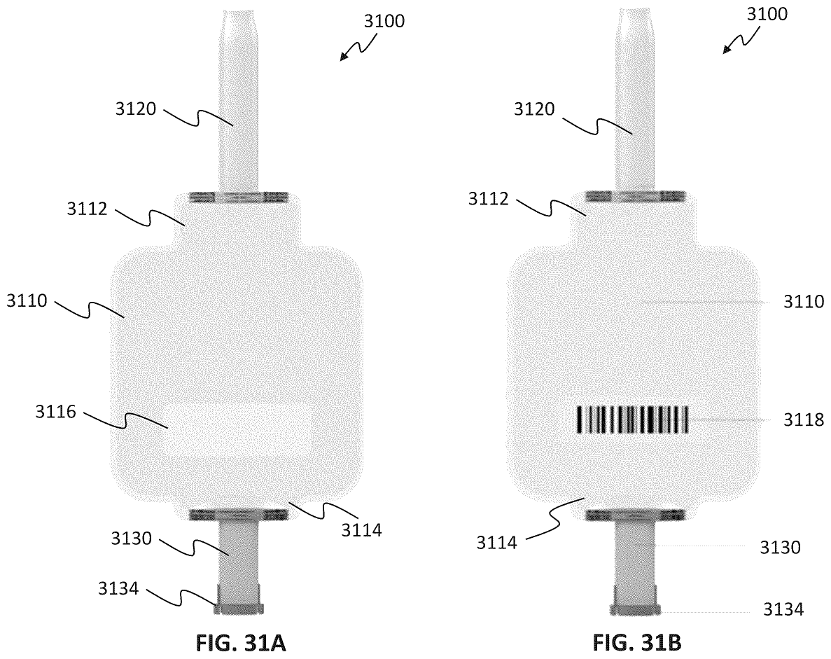

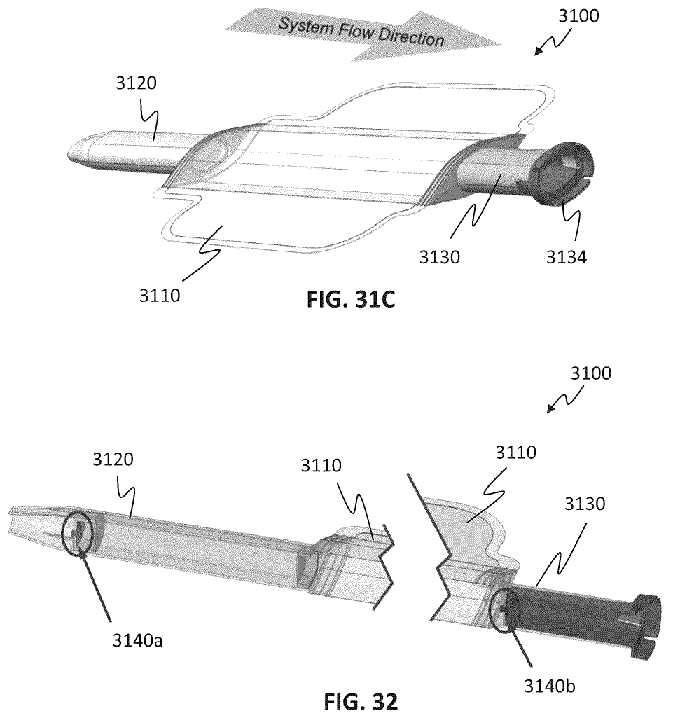

FIGS. 31A-31C depict an example variation of a sampling device 3100. As shown in FIGS. 31A and 31C, the sampling device 3100 may include a compartment 3110 having an inlet portion 3112 and/or an outlet portion 3114. A mouthpiece 3120 may be coupled to the inlet portion 3112 and in fluidic communication with the compartment 3110, such that a user may exhale through the mouthpiece 3120 to deposit a breath sample into the compartment. The sampling device 3100 may, in some variations, include a connector 3130 coupled to the outlet portion 3114 and in fluidic communication with the compartment 3110, such that a sample in the compartment may exit the compartment through the connector 3130. As described in further detail below, the sampling device 3100 may further include a stopper 3134 configured to help prevent escape of the sample from the compartment 3110 and/or help couple the sampling device to a detection device (not shown). As shown in FIG. 31C, in use, a sample may be directed in a “system flow direction” oriented from the mouthpiece 3120 and into the compartment 3110. The sample may subsequently flow from the compartment through the connector 3130 to a detection device (once the sampling device is coupled to the detection device).

Although FIGS. 31A and 31B depict a variation of the sampling device 3100 in which the compartment 3110 has both an inlet and an outlet, it should be understood that in some variations, the compartment 3110 may include only one access opening that functions as both an inlet and an outlet. For example, the compartment 3110 may omit a separate outlet, but include an opening similar that in inlet portion 3112. In this example, the opening in the inlet portion 3112 may be selectively sealable (e.g., allow sealing of the compartment 3110 once a sample is received in the compartment 3110, and allow unsealing of the compartment when the sampling device is coupled to the detection device to permit the sample to be analyzed by the detection device). The mouthpiece 3120 may furthermore be removable (e.g., prior to coupling the sampling device to the detection device) to enable access of the sample contained in the sampling device.

The sampling device 3100 may include one or more features to identify its contents and/or associate the sampling device (and its contents) to a subject. For example, as shown in FIG. 31A, the sampling device 3100 may include a labeling region 3116, which may be, for example, a blank region to receive a label indicative of a subject's identity (e.g., name, code, etc.). The label may be handwritten directly onto the labeling region 3116, include a sticker or decal applied to the labeling region 3116, and/or the like. Additionally or alternatively, as shown in FIG. 31B, the sampling device 3100 may include a sampling device identifier 3118, such as a computer-readable code (e.g., barcode), RFID, serial number, and/or or other suitable identifier for the sampling device. The labeling region 3116 and/or sampling device identifier 3118 may be used to help track the sampling device and identify the subject whose sample is contained within the sampling device.

The sampling device may be sealable with one or more valves, in order to contain a sample. For example, as shown in FIG. 32, the sampling device 3100 may include one or more one-way valves consistent with the system flow direction described above with respect to FIG. 31C, including a first valve 3140a to seal the sampling device 3100 at an inlet (upstream) side, and a second valve 3140b to seal the sampling device 3100 at an outlet (downstream) side. As shown in FIG. 32 and further described below, in some variations the first valve 3140a may be arranged in the mouthpiece 3120 and the second valve 314b may be arranged in the connector 3130. However, the sampling device may be sealed at any suitable points (e.g., at the inlet portion 3112 and/or the outlet portion 3114 of the compartment 3110).

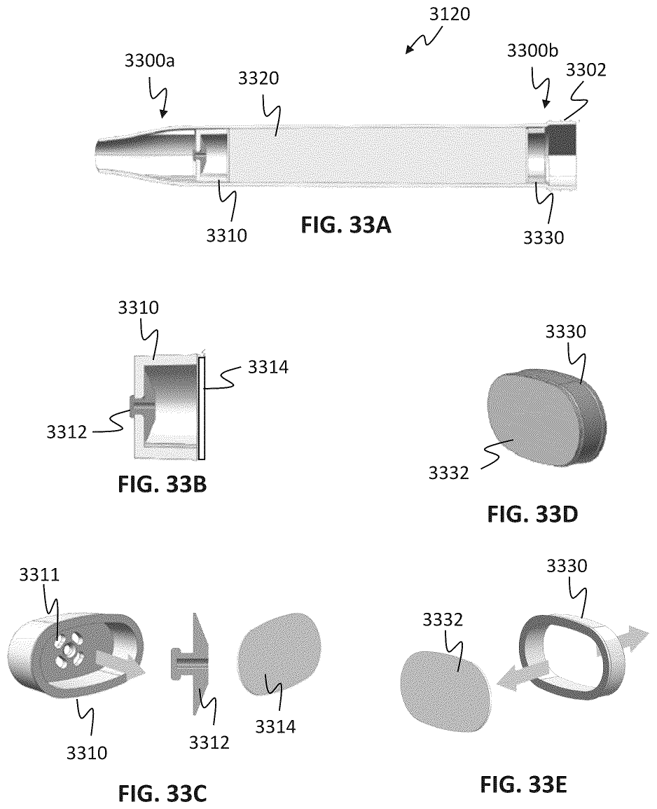

An example variation of a mouthpiece 3120 and its component pieces are shown in FIGS. 33A-33E. The mouthpiece 3120 is primarily described here as being part of the sampling device 3100. However, in some variations, the mouthpiece 3120 may additionally or alternatively couple directly to a detection device (e.g., as shown in FIG. 29 and described above), as part of a detection system omitting the sampling device 3100. As shown in FIG. 33A, mouthpiece 3120 may include a generally tubular structure with an inlet end 3300a and an outlet end 3300b. The inlet end 3300a may be tapered to improve comfort when placed in the mouth of a subject. The outlet end 3300b may be configured to couple to the compartment 3110, and in some variations may include sealing ribs 3302 to improve a fluid-tight seal between the mouthpiece 3120 and the compartment 3110.

In some variations, the mouthpiece 3120 may include one or more valves, one or more filters, and/or a desiccant. For example, FIG. 33B depicts an example variation of an inlet valve carrier assembly, which may be arranged adjacent the inlet end 3300a to receive and begin to process breath exhaled from the user. For example, the inlet valve carrier assembly may be press-fit into the mouthpiece. The inlet valve carrier assembly may include an inlet valve carrier 3310, an inlet valve 3312 arranged within the inlet valve carrier 3310, and a filter 3314 coupled to the inlet valve carrier 3310 (e.g., with epoxy or mechanical interfit). As shown in FIGS. 33B and 33C, the inlet valve carrier 33110 may include an inlet-side wall having openings 3311. The inlet valve 3312 may have a stem that is slidingly engaged in one of the openings 3311, and the inlet valve 3312 may overlie the other openings 3311 such that airflow in the system flow direction (left-to-right as shown in FIG. 33B) will cause the inlet valve 3312 to open and permit airflow through the inlet valve carrier 3310 and filter 3314. The filter 3314, similar to that described above in other mouthpiece variations, may be configured to remove large particles from the exhaled breath of the subject before the breath continues further through the mouthpiece. Similar to that described above, in some variations, the filter may be formed at least in part from a sintered metal material (e.g., aluminum, steel (e.g., stainless steel), titanium, molybdenum, copper, etc. manufactured with sintering techniques). Suitable filter pore size for the filter 3314 may, for example, on the order of about 1 μm or larger. As another example, the filter may include a molecular sieve desiccant, such as an alkaline alumina silicate material.

The inlet valve 3312 may be a one-way or check valve that opens the fluid pathway into the mouthpiece when the subject exhales into the sampling device, but prevents fluid flow in the opposite direction. Thus, the one-way inlet valve enables a subject to provide a breath sample through the mouthpiece, but prevents the subject from inhaling the sampling device's contents. Furthermore, the one-way valve also provides a backstop surface at the inlet side of the sampling device, which urges the contents of the sampling device to exit the compartment at the opposite end (outlet side) of the sampling device when the sampling device is compressed during sample analysis, as further described below.

Additionally, the mouthpiece 3120 may include desiccant 3320 configured to dehumidify the breath sample passing through the mouthpiece 3120. Similar to that discussed above, the desiccant 3320 may include any suitable dehumidifying material such as a silica gel, dehumidifying clay, anhydrous calcium sulfate, and/or other hydrophilic materials. The desiccant 3320 may be shaped to fill the cross-section of the mouthpiece (e.g., elliptical, rectangular with radiused edges, etc.) and extend along a suitable length of the mouthpiece sufficient to dehumidify the sample. As shown in FIG. 33A, the desiccant 33320 may be arranged between the inlet valve carrier 3310 and an outlet filter carrier 3330.

The outlet filter carrier 3330 may include an outlet filter 3332 (e.g., similar to the filter 3314) coupled to a filter ring 3330 (e.g., with epoxy or mechanical interfit). The filter 3332 may perform additional filtering to further remove undesired particles from the breath sample before the sample enters the compartment 3110.

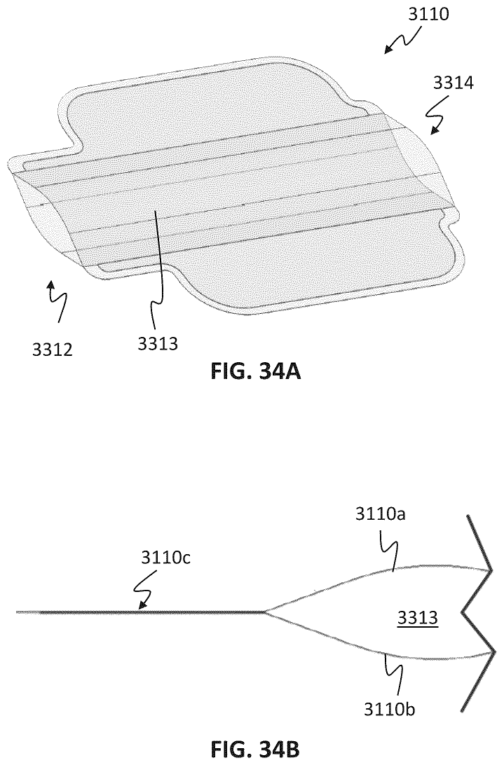

An example variation of a compartment 3110 is shown in FIG. 34A, and a partial cross-section thereof is shown in FIG. 34B. In some variations, the compartment 3110 may be compressible, which may facilitate expulsion of the sample from the compartment 3110 when then compartment 3110 is squeezed, flattened, or otherwise compressed. For example, the compartment 3110 may include a bag. As described above, the compartment 3110 may include an inlet portion 3112 for receiving a mouthpiece (e.g., mouthpiece 3120), and an outlet portion 3114 for receiving a connector for coupling the sampling device to a detection device (e.g., connector 3130). In some variations, the mouthpiece and/or the connector may coupled to the compartment 3110 through RF or heat welding, or other suitable process(es).

The compartment 3110 may be formed in any suitable manner to define a volume for receiving a sample. For example, as shown in FIG. 34A, the compartment 3110 may include a first sheet of material and a second sheet of material opposite the first sheet, where the first and second sheets are sealed together (e.g., heat sealing) to form an edge or partial perimeter of the compartment volume. As shown in FIG. 34A, lateral wings of sheet material may be sealed together to form a generally tubular volume for receiving and storing a sample, though the sheet material may have any suitable shape for forming a volume for receiving a sample. In some variations, the shape of the compartment 3110 may be configured to be flat when empty, then expand outward when receiving a sample. The compartment may include flexible material to facilitate compressibility of the compartment. For example, the compartment 3110 may include a flexible film such as polyethylene, PC, PP, etc. However, it is envisioned that other techniques may be used to form a compartment 3110 that receives a sample (and/or to enable the compartment 3110 to be compressible). The compartment 3110 may include gas impermeable material.

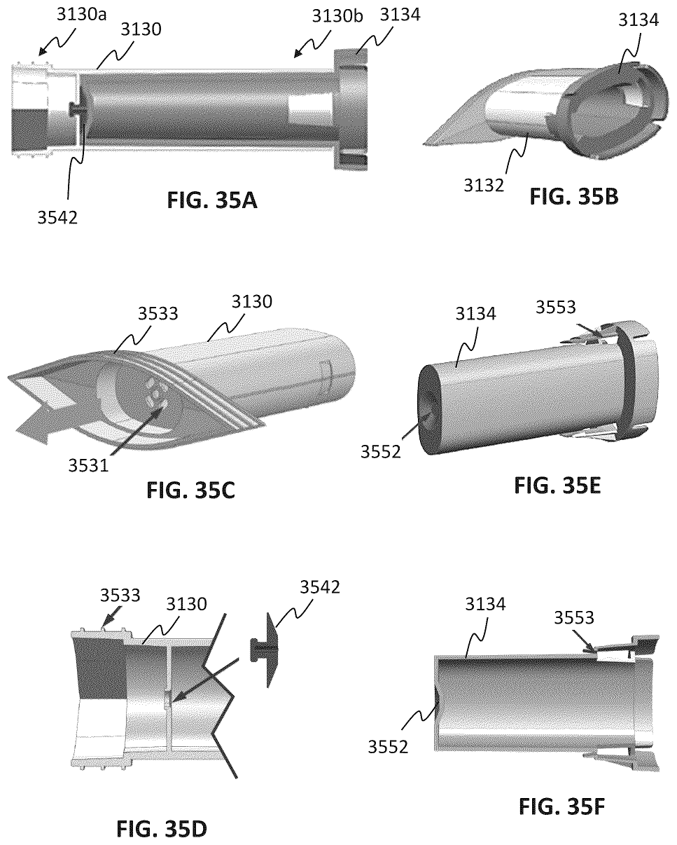

An example variation of a connector 3130 and its component pieces are shown in FIGS. 35A-35F. As described above, the connector 3130 may be configured to couple the sampling device to the detection device (or portion thereof, such as the sensor module). The connector 3130 may include a generally tubular structure with an inlet end 3130a and an outlet end 3130b. The inlet end 3130a may be configured to couple to the compartment 3110, and may in some variations include one or more sealing features such as sealing ribs 3533 (shown in FIGS. 35C and 35D) to help improve a fluidic seal between the compartment and the connector. The outlet end 3130b may be configured to couple to a detection device (or portion thereof, such as the sensor module), such as with engagement features (e.g., snap-fit, etc.).

The connector 3130 may include one or more valves, such as outlet valve 3542, to help seal the contents of the compartment. The connector 3130 may include, for example, a wall having openings 3531 as shown in FIG. 35C. Like the inlet valve 3312 in the mouthpiece described above, the outlet valve 3542 may have a stem that is slidingly engaged in one of the openings 3531, and the outlet valve 3542 may overlie the other openings 3531 such that airflow in the system flow direction (left-to-right as shown in FIG. 35A) will cause the outlet valve 3542 to open and permit airflow through the connector 3130 and into the detection device (once coupled to the detection device).

In some variations, the sampling device may further include a stopper 3134, which may function to help maintain the closed position of the outlet valve 3542 prior to coupling the sampling device to the detection device. As shown in FIGS. 35A and 35B, the stopper 3134 may be a generally tubular structure that engages telescopically with (e.g., inserted into) the connector 3130. The engagement may be secured or locked in any suitable manner, such as mechanical interfit (e.g., snap-fit, dimensional interferences), latches, etc. For example, the stopper may include engagement features 3553 (e.g., flexing arms) that engage with corresponding engagement features on the connector 3130, such as in a snap-fit manner. At an inlet end of the stopper 3134, the stopper 3134 may include a valve contour 2552 that is sized and shaped to hold the outlet valve 3542 in a closed position in the connector 3130. Accordingly, when the stopper 3134 is engaged with the connector 3130, the outlet valve 3542 may be maintained in a closed position, thereby sealing the contents (e.g., breath sample) within the sampling device. In some variations, the stopper 3134 may have an outlet end configured to ease removal for the stopper 3134 from the connector 3130 (e.g., flange, flared edge, ridges etc.). Once the stopper 3134 is removed, the outlet valve 3542 may be opened and/or the outlet end 3130b of the connector may be exposed and free to couple with the detection device.

Like the inlet valve 3312 in the mouthpiece, the outlet valve 3132 may be a one-way or check valve that opens the fluid pathway from the compartment. In some variations, the outlet valve 3132 may be configured to open under only high pressure (high crack pressure) when the compartment is compressed, but not open during other normal use (e.g., transport, manual handling when obtaining a sample from a subject, etc.). Accordingly, the outlet end of the sampling device may be sealed at least in part by a combination of an outlet valve 3132 having a high crack pressure and placement of the stopper 3134. However, in some variations, the outlet valve 3132 may have a lower crack pressure and the stopper 3134 may alone be sufficient to seal the outlet end of the sampling device.

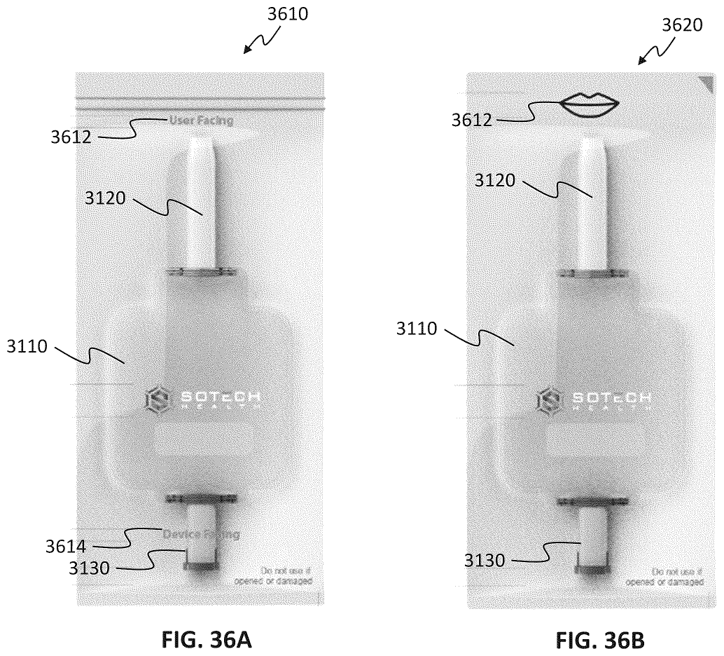

As described above, in some variations the sampling device has a “system flow direction” in which a sample is intended to move through sampling device. Accordingly, it may be important to indicate the inlet portion and/or the outlet portion of the sampling device through labeling on packaging for the sampling device and/or directly on the sampling device. For example, packaging 3610 (e.g., pouch or sealed overwrap) may include an inlet indicator 3612 located in a region proximate the mouthpiece 3120, and/or an outlet indicator 3614 located in a region proximate the connector 3130. As shown in FIG. 36A, the inlet indicator 3612 and/or the outlet indicator 3614 may include text (e.g., “user facing”, “mouthpiece”, “U”, “D”, “device facing”, etc.). Additionally or alternatively, the inlet indicator 3612 and/or the outlet indicator 3614 may include a graphic icon (e.g., lips, face, icon representative of a device, etc.). Furthermore, in some variations a similar inlet indicator and/or outlet indicator may be on the sampling device itself. For example, an inlet indicator and/or outlet indicator (e.g., text and/or graphic icon) may be printed on or molded into material of the sampling device, or applied to the sampling device through a decal, etc.

Sample Extraction from Sampling Device

As described above, a sampling device may receive and store a sample (e.g., breath sample) from a user while detached from a detection device. After storing a sample, a sampling device may be transported to a suitable location and/or held until a suitable time for analysis with a detection device. For example, the sampling device may be coupled to a detection device to permit fluidic communication between the compartment of the sampling device and the sensor module in the detection device. In some variations, one or more components may be removed from the sampling device (e.g., stopper such as stopper 3134 described above) and/or the detection device to facilitate the coupling and/or fluid communication between the sampling device and detection device. The sample may then flow from the sampling device to the sensor module for analysis.

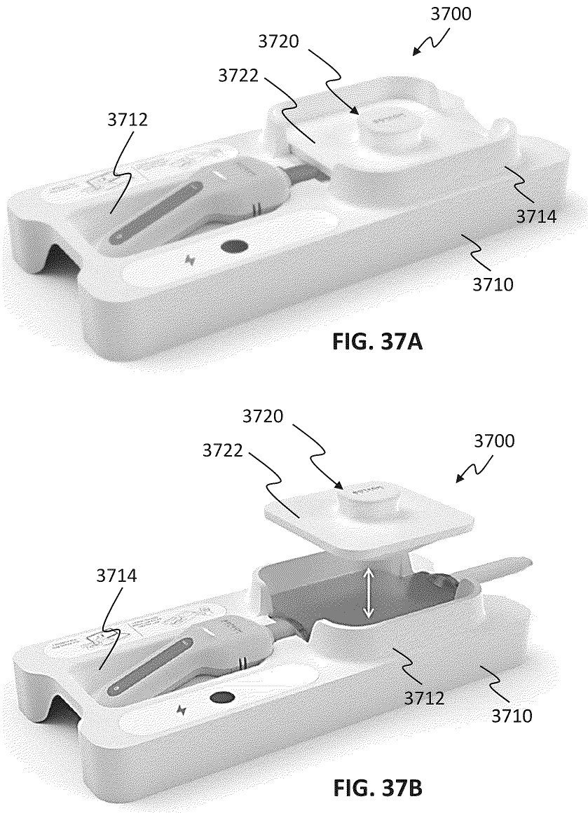

In some variations, a stored sample may be obtained from the sampling device with the aid of a sample extractor. An example variation of a sampling extractor 3700 is shown in FIGS. 37A and 37B. As shown in FIG. 37A, a sampling extractor 3700 may include a base 3710 and a press 3720 configured to compress a sampling device (e.g., a flexible, compressible compartment of the sampling device) against the base 3710, thereby urging or expelling a stored sample out of the sampling device. Generally, the base 3710 and/or the press 3720 may include suitable rigid materials to surround the sampling device on opposite sides, such that urging the base 3710 and the press 3720 toward each other (with a sampling device placed therebetween) causes the stored sample to exit the sampling device in a system flow direction toward an outlet of the sampling device (e.g., due to the one-way valves as described above).

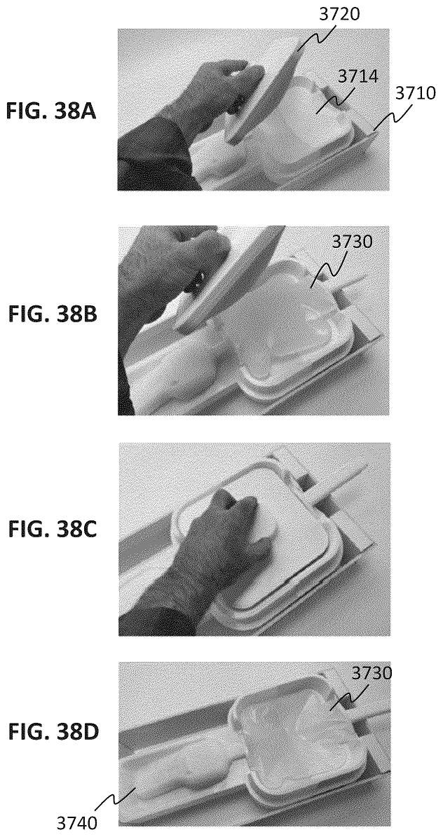

The base 3710 may include a sampling device cavity 3714 sized and shaped to receive a compressible portion of a sampling device. For example, in some variations, the cavity 3714 may include a contoured cavity, to accommodate an expanded sampling device. In some variations, the contoured cavity may have one or more sloping sides tapering to a radiused point (e.g., central point), as shown in FIG. 38A. For example, the cavity 3714 may have an inverted cone or pyramidal shape. As another example, the contoured cavity may be bowl-shaped (e.g., elliptical or other arcuate cross-section) or have any other suitable contour. Alternatively, the cavity 3714 may have a planar bottom surface against which the sampling device may be pressed.

When a sampling device is placed in the cavity 3714, the outlet end of the sampling device (e.g., connector such as 3130 described above) may be accessible to receive the expelled sample. As shown in FIG. 37A, the sampling device cavity 3714 may include sidewalls to help locate placement of the sampling device in the cavity 3714 and/or help contain the sampling device in the cavity 3714.

The press 3720 may include a pressing member 3722, including a pressing surface configured to oppose the surface of the sampling device cavity 3714. In some variations, the pressing surface may be contoured in a manner matching or corresponding to the contour of the sampling device cavity 3714. When a sampling device is placed between similarly-contoured surfaces of the cavity 3714 and the pressing surface of the press 3720, the pressure exerted by the press 3720 on the sampling device may advantageously be more uniform across the sampling device and consistent throughout use of the press 3720. In some variations, as shown in FIGS. 37A and 37B, the press 3720 may include a handle (e.g., knob) coupled or integrally formed with the pressing member 3722, which a user may grasp and use to manipulate the press 3720. In some variations, the handle may include one or more features to improve grip for a user, such as ergonomic and/or textural features (e.g., finger grips, flared edges, high friction materials, ribs, etc.). Furthermore, in some variations, the sampling device cavity 3714 and/or the press 3720 may include one or more alignment features (e.g., keyed features, grooves, etc.) that may help guide the relative positioning and/or movement of the press 3720 and the sampling device cavity 3714. In some variations, the press 3720 may be configured to be actuated manually be a user, though in some variations the press 3720 may additionally or alternatively actuated automatically or semi-automatically (e.g., by a robotically-controlled actuator, etc.).

In some variations, the base 3710 may further include a detection device cavity 3714 configured to receive a detection device, such that the detection device and sampling device may be placed in the base 3710 while coupled to another. The detection device cavity 3714 and/or the sampling device cavity 3712 may be molded specifically to the shape of the detection device and sampling device, respectively, such that the detection device and/or sampling device may be snug or otherwise secure in their respective cavities during operation of the sample extractor.

As described above, the sampling device 3700 shown in FIGS. 37A and 37B includes a base 3710 with a sampling device-receiving cavity (negative space) that is complementary to a projecting pressing surface (positive feature) on the press 3720. However, it should be understood that in other variations, the locations of the cavity and the projecting pressing surface may be swapped. For example, a sample extractor may instead include a base with a projecting surface (e.g., hill-shaped positive feature) that is complementary to a sampling device-receiving cavity (negative space) on the press 3720. Furthermore, in some variations, each of the sampling device receiving cavity and the press may include a combination of projecting features and cavities (e.g., undulating surfaces, etc.) for use in compressing a sampling device therebetween.

FIGS. 38A-38D depicts an example method of use of the sample extractor shown in FIGS. 37A and 37B. FIG. 38A depicts the sample extractor 3700 including a base 3710 with a sampling device cavity 3714, and a press 3720. As shown in FIG. 38B, an expanded compartment of the sampling device 3730 including a sample may be placed in the sampling device cavity 3714. A user may manually locate the press 3720 over the expanded compartment and sampling device cavity, and then urge the press 3720 toward the base 3710 as shown in FIG. 38C. This “sandwiching” action thereby compresses the compartment of the sampling device, expelling the stored sample from the compartment out an outlet end. If a detection device 3740 is fluidically coupled to the sampling device 3730 at the time of this compression shown in FIG. 38C, the expelled sample may then be communicated to a sensor module in the detection device 3740.

Although FIGS. 38A-38D depict a method of manual compression using the sample extractor, in some variations a similar compressive technique may be performed automatically or semi-automatically, such as with a robotically-controlled actuator.

### Sterility

As described herein, in some variations, a detection system may include a detection device and a mouthpiece (or other sampling device). The detection device may be configured to analyze multiple sample (e.g., until a sensor module reaches the end of its usable lifetime, or until is has been used a predetermined number of times), though each sample may be obtained from a different subject via a different mouthpiece. In some variations, the detection device may be sanitized (e.g., with alcohol wipes, UV sterilization, etc.) between uses to reduce cross-contamination between different subjects.

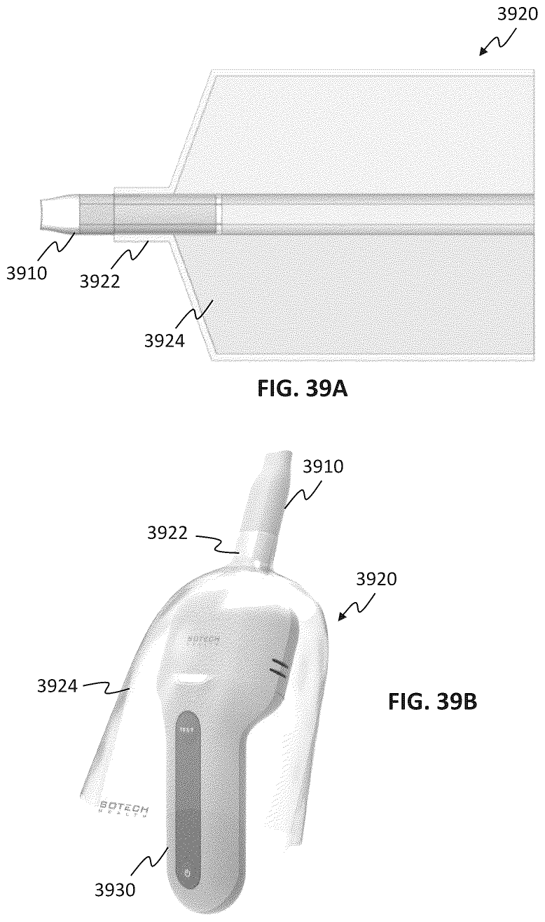

Additionally or alternatively, the detection system may include one or more sterile interfaces to help protect the detection device between uses by different subjects. For example, FIG. 39A illustrates an example variation of a sheath 3920 that may be attached to a mouthpiece 3910. The sheath may include a neck portion 3922 configured to engage the mouthpiece 3910, and a skirt portion 3924 configured to accommodate a detection device 3930 held underneath the skirt portion 3924. As shown in FIG. 39B, the skirt portion 3924 covers and shields the detection device 3930, and the neck portion 3922 helps secures the sheath 3920 in place over the detection device 3930. In this configuration, the sheath 3920 also defines and separates a non-sterile region (region above the sheath 3920 as shown in FIG. 39B, including the mouthpiece 3910) from a “sterile” region (region below the sheath 3920). When the mouthpiece 3910 has been used to gather a sample from a subject and is ready to be disposed, the sheath 3920 may then be everted to contain the non-sterile surface of the sheath 3920 on an internal surface, thereby protecting handlers of the used mouthpiece from contamination.

In some variations, the sheath may be pre-attached to the mouthpiece 3910. For example, the neck portion 3922 of the sheath may be coupled to the mouthpiece 3910 (e.g., through one or more fasteners such as epoxy, RF or heat welding, etc.). As another example, the sheath may be integrally formed with and attached to the mouthpiece 3910 (e.g., overmolded sheath, or sheath otherwise integrally molded as a membrane extending from the mouthpiece, etc.). In some variations, the pre-attached sheath may be packaged in a compact manner with the mouthpiece (e.g., rolled and/or folded, such as against the mouthpiece), then unfurled to the configuration shown in FIG. 39A. In some variations, the skirt portion 3924 may be inverted in the compact packaged configuration, such that the skirt portion 3924 may be everted over the mouthpiece 3910 and/or detection device 3930 for use in shielding the detection device.

Alternatively, the sheath 3920 may be provided separately from the mouthpiece and then manipulated to engage the mouthpiece. For example, the sheath 3920 may have a tapering neck portion 3922 such that the skirt portion 3924 may be slipped over the mouthpiece 3910 and pulled down until the tapering neck portion 3922 interferes with the diameter of the mouthpiece 3910, thereby engaging the mouthpiece 3910 to form substantially the configuration shown in FIG. 39A. In some variations, a seal may furthermore be formed between the interface of the sheath 3920 and the mouthpiece 3910 (e.g., with tape, a surrounding sealing collet or suitable connector, etc.) to improve the shielding function of the sheath 3920.

In some variations, the material of the sheath may include a suitable waterproof material, such as high density polyethylene or silicone, though other variations may include other suitable materials. Furthermore, it is contemplated that in some variations, the sheath may have other suitable shapes (e.g., triangular) not shown in FIGS. 39A and 39B.

### Mobile Application

As described elsewhere herein, in some variations, a detection device may be communicatively coupled with one or more computing devices, where at least one of the computing devices may execute a mobile application with functionality that complements operation of the detection device. The mobile application may, for example, provide instructions for using the detection device, provide status of the detection device, communicate test results following sample analysis, communicate alerts, enable access to user and/or test data, and/or the like.

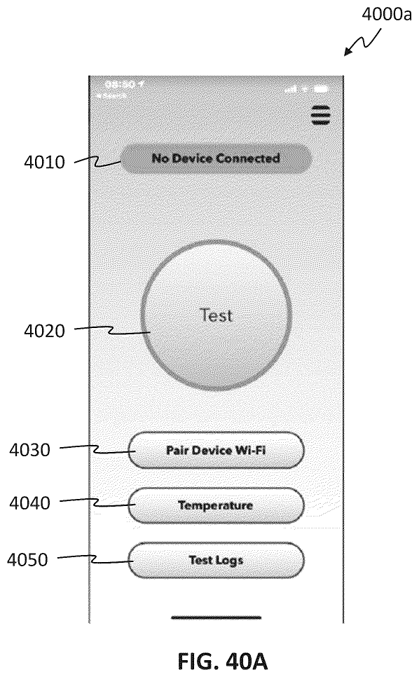

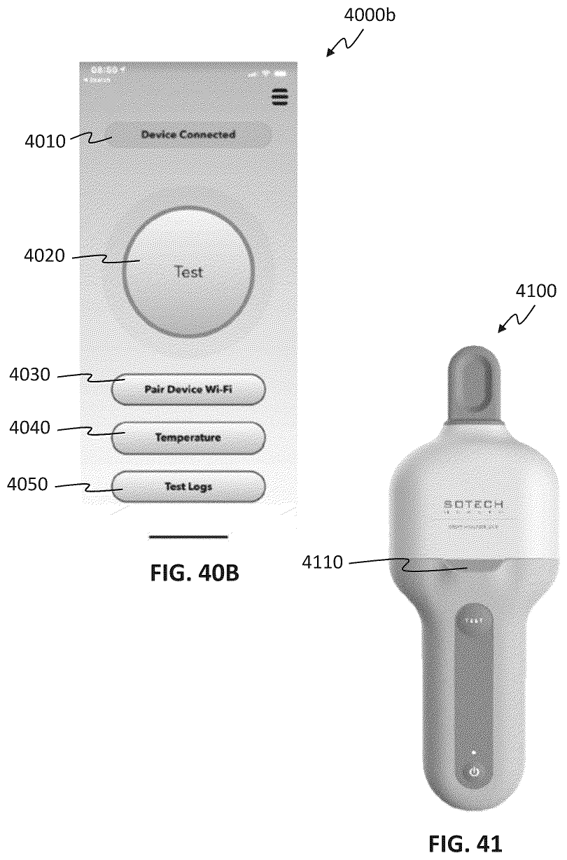

For example, FIG. 40A illustrates an example variation of a graphical user interface (GUI) 4000a of a mobile application executed on a computing device (e.g., mobile phone) for use with a detection device with a mouthpiece (e.g., similar to the detection device described above with reference to FIGS. 29A and 29B). The GUI 4000a may, for example, function as a home screen that is displayed when the mobile application is first opened. In some variations, once the mobile application is opened on a computing device, the computing device may automatically begin scanning for nearby detection devices (e.g., for connection via Bluetooth or other wireless communication modalities) with which to pair. Additionally or alternatively, pairing to one or more detection devices may be manually performed, and may be initiated through the GUI 4000a (e.g., pairing via Wi-Fi through pairing button 4030). The GUI 4000a may furthermore display a device connection status 4010 (e.g., indicating “No Device Connected”, “Scanning For Device”, “Device Connected”, etc.). In some variations, 4000a may include a test initiation button 4020 or other suitable interactive icon for initiating a test. In some variations, the GUI 4000a may include other suitable menu items, such as a temperature logging option (e.g., temperature logging button 4040) to enable recordation of a user's temperature (and/or other user symptoms such as heart rate, oxygen saturation, etc.), or options to view previous test data (e.g., test log button 4050).

FIG. 40B illustrates an example variation of a GUI 4000b, which is similar to GUI 4000a described above, except that the device connection status 4010 in GUI 4000b is depicted as indicating (through text and/or color change) a successful pairing to a detection device, such as through Bluetooth. In some variations, the paired detection device may additionally or alternatively indicate a successful pairing to a computing device through the mobile application. For example, FIG. 41 depicts a detection device 4100 (e.g., similar to the detection device described above with reference to FIGS. 29A and 29B) including an indicator 4110 that may illuminate with a predetermined color (e.g., blue) and/or timing pattern to communicate that the detection device 4100 is paired with a computing device.

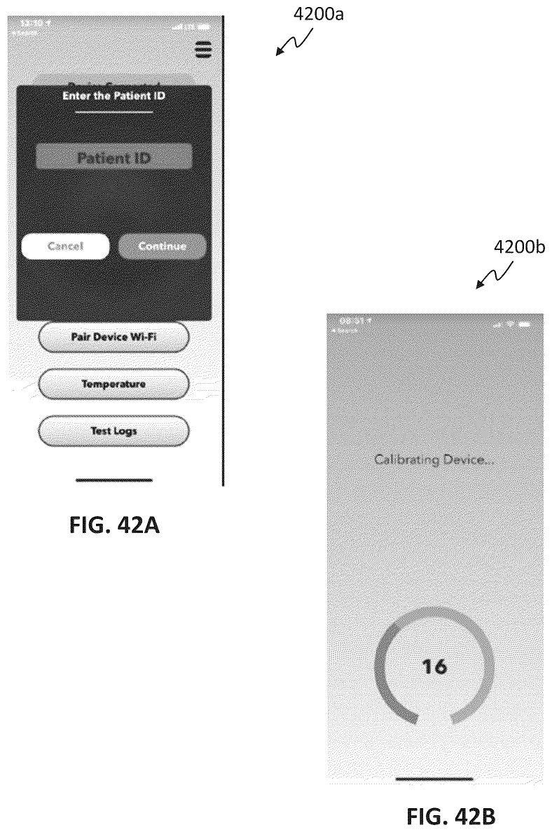

As described above, a test or sample analysis may be initiated through the mobile application, such as by a user pressing the test initiation button 4020. FIG. 42A illustrates an example variation of a GUI 4200a that may appear in response to initiation of a test. For example, GUI 4200a may prompt entry of one or more patient identifiers (e.g., name, serial number, medical record number, etc.) to be used to associate a user (e.g., patient) of the detection device with test results of a provided sample. One or more additional prompts may, in some variations, provide further instructions to a user for operating the detection device to perform a test.

In some variations, the mobile application may provide an indication of detection device status as the detection device prepares for a test. For example, FIG. 42B illustrates an example variation of a GUI 4200b that indicates that the detection device is calibrating prior to receiving a breath sample. As shown in FIG. 42B, the GUI 4200b may include a countdown timer that visually indicates progress of calibration and/or other device actions in preparation for a test. The countdown timer may include a numerical timer and/or other suitable visual indicator for communicating such information. During this time, a user may place his or her mouth on the mouthpiece of the detection device and prepare to exhale into the mouthpiece to provide a breath sample.

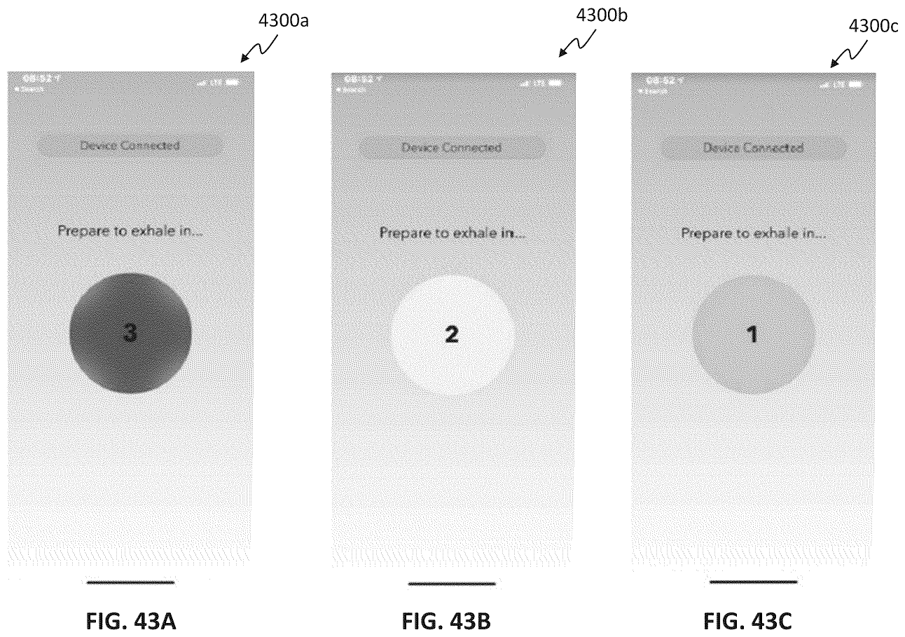

In some variations, the mobile application may provide further instructions to a user for providing a breath sample, such as a countdown timer such as that in example GUIs 4300a-4300c shown in FIGS. 43A-43C. For example, in GUIs 4300a-4300c, a numerical and/or color-coded timer (e.g., an icon progressing from red, to yellow, to green) may provide a countdown for instructing a user to exhale into the mouthpiece of the detection device to provide a breath sample. During this time, if the user may place his or her mouth on the mouth on the mouthpiece if he or she has not done so already. Although GUIs 4300a-4300c depict the final three seconds of a countdown timer, it should be understood that the indicated countdown period may have any suitable duration (e.g., 5 seconds, 10 seconds).

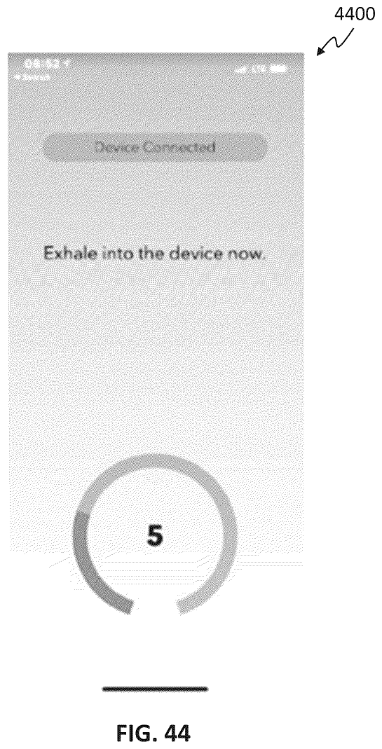

The mobile application may, in some variations, provide instructions to guide a user while he or she is providing a breath sample. For example, FIG. 44 illustrates an example variation of a GUI 4400 that may provide a numerical and/or color-coded or other visual timer (e.g., progress ring) that indicates when a sufficient breath sample volume has been obtained through the mouthpiece. In GUI 4400, a numerical timer (e.g., countdown) may correspond to a visual progress ring that becomes filled or completed as a breath sample is being obtained. Text and/or audio instructions (e.g., “Exhale into the device now.”) may furthermore be provided through the GUI 4400. Accordingly, in some variations, a user may be expected to exhale into the mouthpiece until the timer(s) have elapsed and a successful sample volume is obtained. In some variations, another GUI may provide confirmation that sufficient breath sample has been obtained.

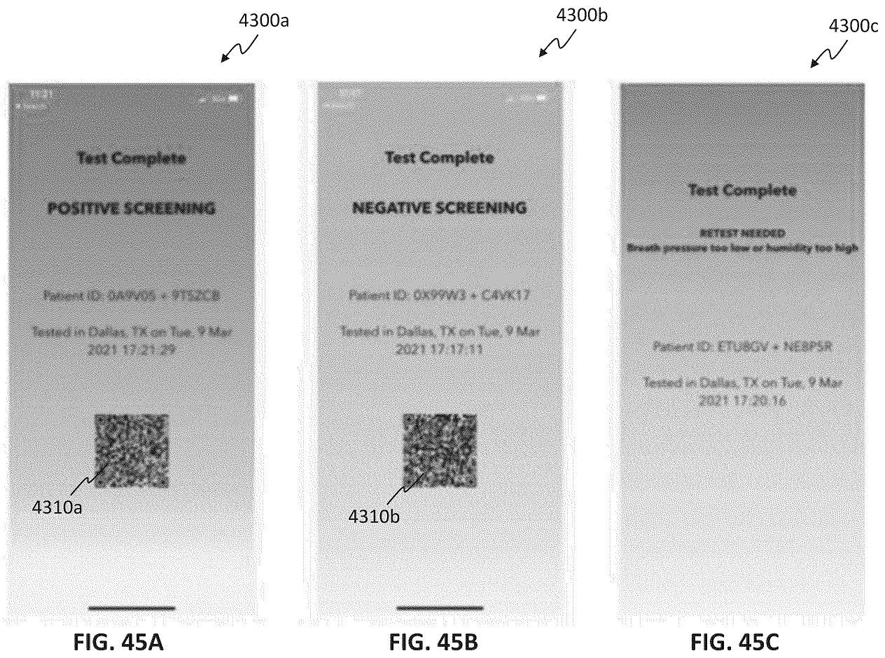

In some variations, the mobile application may provide an indication of one or more test results based on analysis of the received breath sample. For example, FIG. 45A illustrates an example variation of a GUI 4300a that indicates test results including that the test was completed, that the test resulted in detection of the target analyte (e.g., “Positive Screening”), patient identification information, and/or test details (e.g., date, time, place, etc.). One or more of such test results may be furthermore encoded in a computer-readable code 4310A (e.g., QR code, other bar code, etc.) that can be scanned for accessing and/or recording the test results. As another example, FIG. 45B illustrates an example variation of a GUI 4300b that indicates test results including that the test was completed, that the test did not result in detection of the target analyte (e.g., “Negative Screening”), patient identification information, and/or test details (e.g., date, time, place, etc.). Like in GUI 4300a, one or more of such test results may be furthermore encoded in a computer-readable code 4310b. As another example, FIG. 45C illustrates an example variation of a GUI 4300c that indicates test results including that the test was completed, that the test resulted in one or more test errors (e.g., “Breath pressure too low or humidity too high” as shown in FIG. 45C), indicates that the test should be repeated (e.g., “Retest needed”), patient identification information, and/or test details (date, time, place, etc.). In some variations, GUI 4300c may include a computer-readable code that encodes test results, similar to that shown in GUI 4300a and GUI 4300b. In some variations, the mobile application may furthermore display a suitable GUI that allows any of such test results to be forwarded or otherwise shared (e.g., emailed to the user, emailed to a testing facility or other administrator, emailed to health authorities, etc.).

### Methods for Detecting VOCs

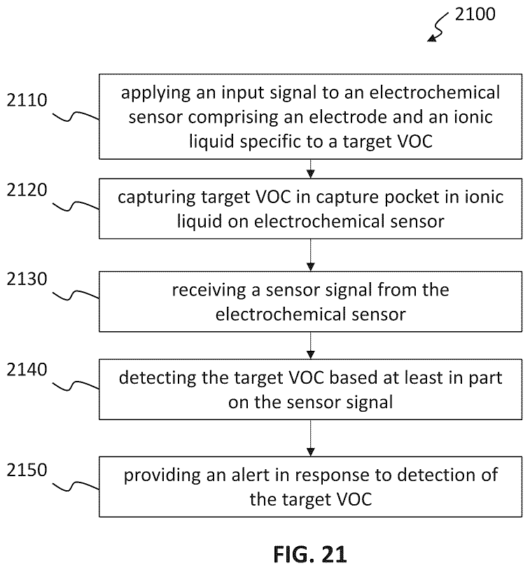

Various methods for detecting one or more target analytes (e.g., target VOCs) may be performed using systems such as those described herein. For example, FIG. 21 depicts a method 2100 for detecting one or more target VOCs including applying an input signal to an electrochemical sensor 2110 including an electrode and an ionic liquid (e.g., RTIL) specific to a target VOC, capturing a target VOC in one or more cavities in the ionic liquid 2120, receiving a sensor signal from the electrochemical sensor 2130, and detecting the target VOC based at least in part on the sensor signal 2140. For example, as described above, applying an input signal (e.g., DC signal) to an electrochemical sensor may result in polarization of the RTIL, which stretches RTIL bonds to create one or more cavities for capturing the target VOC within the RTIL. The cavity or cavities may be arranged between anionic groups of adjacent layers of the RTIL, where the anionic groups are specific to the target VOC in a puzzle piece-like manner. If present in the environment around the electrochemical sensor, the target VOC is captured in the cavity or cavities so as to diffuse through the RTIL toward the electrode. The capture of a target VOC may be detectable as a change in current (e.g., difference between a new current in the sensor signal and a baseline current, ratio between a new current in the sensor signal and a baseline current) when a voltage potential is applied across the electrode. Furthermore, the amount or concentration of the target VOC may also be determined based on the magnitude of the change in current. The method may further include providing an alert in response to the detection of the target VOC 2150, such as by indicating presence and/or estimated quantity of the target VOC on a user interface of the detection device and/or communicating the same to a peripheral device or other computing device. In some variations, the detection device may provide a detection signal whose strength corresponds to, for example, concentration and/or proximity of the target VOC to the detection device.

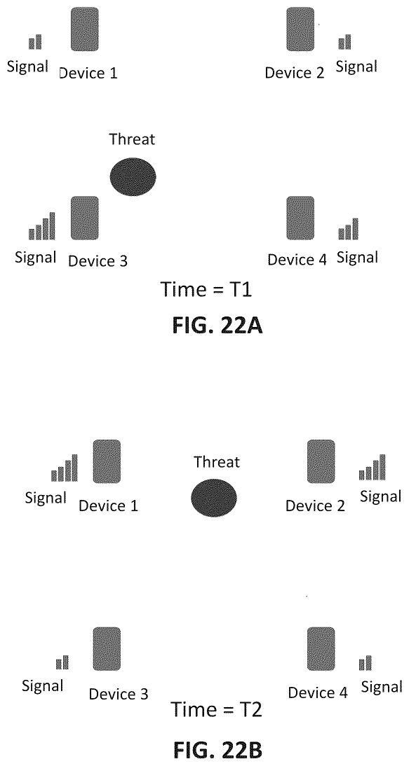

In some variations, multiple detection devices may be used to obtain additional information. For example, multiple detection devices may communicate with each other and/or peripheral devices through one or more wireless communication modules as described above (e.g., Bluetooth, Wi-Fi) and include location information. Each detection device may be loaded with software to enable the detection device to articulate its position to itself and other detection devices and/or peripheral devices, to allow for the tracking and triangulation of VOCs and/or other threats. In some variations, a detection device may periodically or intermittently scan for other nearby detection devices to set up a custom network of communication. For example, as shown in the illustrative schematic of FIGS. 22A and 22B, multiple devices may be placed in various locations, such as one detection device at each corner of a room (Devices 1-Devices 4), and may communicate with each other. At time T1 shown in FIG. 22A, a threat carrying a detectable VOC may be closest to Device 3. Accordingly, at time T1, the detection signal from Device 3 may be the strongest out the four pictured devices, while the detection signal from the other devices may be weaker corresponding to distance (e.g., the detection signal from Device 2 may be weakest). As the threat moves across the room, the detection signal strengths from the various detection devices change. For example, at time T2 shown in FIG. 22B, the threat is closer to Devices 1 and 2, and the detection signals from Devices 1 and 2 may be stronger than those from Devices 3 and 4. The change in detection signal strengths among the detection devices may thus allow the devices to triangulate how the threat is moving within the room and identify where the threat is located. The triangulation detections may be performed as frequently as desired to obtain a suitable understanding of the environment. For example, the calculations may be performed one or more times per second (e.g., 1 Hz, up to 3 Hz, up to 5 Hz, etc.) to obtain real-time or near real-time information about the threat's movement.

In some variations, methods for detecting threats may utilize the wireless communication modules to track other possible threats. For example, a detection device may have software that enables scanning of nearby Wi-Fi and/or Bluetooth signal SSIDs to identify any other nearby computing devices that may be attempting to communicate with or pair with other systems. For example, any device outputting a Bluetooth or Wi-Fi signal actively advertises what type of mating apparatus it is trying to pair to (e.g., a person's smartphone is constantly searching for the person's home Wi-Fi, or perhaps the person's Bluetooth headphones or other device). Accordingly, a detection device such as that described herein may be configured to identify the outputted pairing signal from a nearby computing device and derive information from the pairing signal. As an illustrative example, a detection device may detect an outputted pairing signal from a nearby smartphone seeking to re-pair with Wi-Fi associated with a particular home address. By analyzing the outputted pairing signal, the detection device may interpolate that the owner of the smartphone emitting the pairing signal likely lives in a home at that home address. Accordingly, this signal “sniffing” capability of the detection device may augment the threat detection capabilities and enable the detection device to not only detect a target VOC, but also gain information on a human transporter carrying that target VOC.

As described above, the detection devices may be used to monitor and/or track various target analytes in a variety of applications. For example, some methods for detecting VOCs may involve detecting a target VOC that is characteristic of an explosive (e.g., C-4 explosive, gunpowder, etc.), drug, or other substance. As another example, some methods for detecting VOCs may be involve detecting a target VOC that is characteristic of a health state of a user. Specific examples by way of illustration are described in further detail below.

### EXAMPLES

The sensor can be used to detect a number of different analytes or analyte classes that are useful in applications such as air monitoring, biomedical diagnostics, industrial processes, and in security and occupational health. In some variations, the detection device may include at least one electrochemical sensor that detects a VOC characteristic of an explosive or an explosive mixture. Such a VOC could, for instance, be a taggant, or a volatile chemical added to an explosive to aid in detecting the presence of a bomb. As non-limiting examples, 2,4-dinitrotoluene; 2,6-dinitrotoluene; 1-ethyl-2-nitrobenzene; and/or cyclohexanone might be present in C-4 explosive, and are therefore VOCs characteristic of an explosive. In some variations, the VOC is characteristic of a plastic explosive. In some variations, the VOC is characteristic of Composition C-4 (C-4). In some variations, the VOC is characteristic of gunpowder. Specificity for a target VOC characteristic of an explosive is achieved by modulating the RTIL of the sensor as well as the electrode input signal as previously described.

In some variations, the electrochemical sensor detects a biomarker VOC. A biomarker is a quantifiable characteristic of a particular biological process that may indicate a particular health state. For example, in certain diseases, a metabolic pathway, such as lipid peroxidation, may be altered such that a unique signature of VOCs is produced (i.e., a unique mixture of aliphatic hydrocarbons). The electrochemical sensor may be applied, for example, in the health care industry, at the patient's bedside, or in self-administered diagnostics, etc. In some variations, the biomarker is a human biomarker. In some variations, the target VOC is one or more biomarker associated with a health state (e.g., medical condition). In some variations, the medical condition is a human medical condition, such as presence of COVID-19. For example, detection of aliphatic hydrocarbons and inorganic gases released by the human body upon up-regulation or down regulation of metabolic processes can be correlated to the presence or absence of COVID-19. The selection of RTIL is done based on the extent of interaction between functionalized imidazolium-based cation and fluorinated anion. Inorganic gases like NOx are released by the metabolic pathways and can be easily detected in the human breath. For example, NOx interacts with fluorinated functionalized imidazolium compound present in the RTIL at the sensor surface and causes a measurable change in current as described above. This combination of NOx and imidazolium-based RTIL can be tuned for specificity for COVID-19 relevant targets.

In some variations, the electrochemical sensor detects a VOC characteristic of use of a drug (e.g., VOC produced by the body as a result of regulation metabolic pathways). In some variations, the drug is a cannabinoid, alcohol, or an opioid. In some variations, the drug is an opioid. In some variations, the drug is fentanyl.

### Example 1: Selection of RTILs was Optimized for Each Target VOC

The optimal RTIL for detection of each VOC was determined under different sensing conditions. Specifically, 1 ppb and 800 ppb of VOC solution was prepared. 3 uL of RTIL was dispensed on the sensor surface and a baseline reading was recorded in the absence of VOC. 1 ppb VOC was added inside a sensing chamber and current response through chronoamperometry (CA) was measured. The signal was recorded, and the chamber was cleaned with N2 to remove any residual VOC before next concentration was tested. The procedure was repeated for 800 ppb VOC and the signal change (relative to 1 ppb VOC) was recorded.

Table 1 shows target VOCs characteristic of an explosive, the optimal RTIL used to selectively detect each VOC, and the lower limit of detection.

TABLE 1
VOCs characteristic of an explosive

| Target Analyte | RTIL Used | Limit of Detection |
|---|---|---|
| 2,4-dinitrotoluene | BMIM-BF₄ | 1 ppb |
| 2,6-dinitrotoluene | BMIM-BF₄ | 1 ppb |
| 1-ethyl-2-nitrobenzene | BMIM-BF₄ | 1 ppb |
| Cyclohexanone | BMIM-Cl | 1 ppb |
| Sulfur dioxide (gun powder) | BMIM-BF₄ | 100 ppb |

Table 2 shows a target VOC associated with use of a drug, the optimal RTIL used to selectively detect each VOC, and the lower limit of detection.

TABLE 2
VOCs associated with use of drugs

| Drug | RTIL Used | Limit of Detection |
|---|---|---|
| Fentanyl | EMIM-TF₂N | 10 ppb |

Table 3 shows target VOCs characteristic of the presence of COVID-19, and the optimal RTIL used to selectively detect each VOC.

TABLE 3
VOCs characteristic of the presence of COVID-19

| VOC | RTIL Used |
|---|---|
| NOx | EMIM-BF₄ |
| Aliphatic hydrocarbon | EMIM-OTf |

### Example 2: Sensitivity of a BMIM[BF₄]-Based Sensor Toward VOCs Characteristic of an Explosive was Analyzed

In order to test the ability of the sensor to detect VOCs at various concentrations, and to test the ability of the sensor to differentiate between those concentrations, [BMIM]BF₄ was used as the RTIL. Three VOCs characteristic of an explosive were analyzed: 2,4-dinitrotoluene (2,4-DNT); 2,6-dinitrotoluene (2,6-DNT); and 1-ethyl-2-nitrobenzene (ENB). Briefly, 3 μL of [BMIM]BF₄ was dispensed onto the sensor electrodes, and the sensor was placed inside a test chamber at 25° C. The baseline measurement was recorded without any VOC present. A chronoamperometry scan was run at a fixed potential, and the current was recorded. The fixed potential allows for binding to occur specifically to a given VOC. Next, VOC samples were placed into the test chamber at concentrations of 1 ppb and 800 ppb. At each concentration of VOC, a negative potential was applied, allowing the diffused VOC species to reach the electrode, selectively interacting with the ionic species of the RTIL layer. The current was measured. The setup was cleaned before subsequent readings.

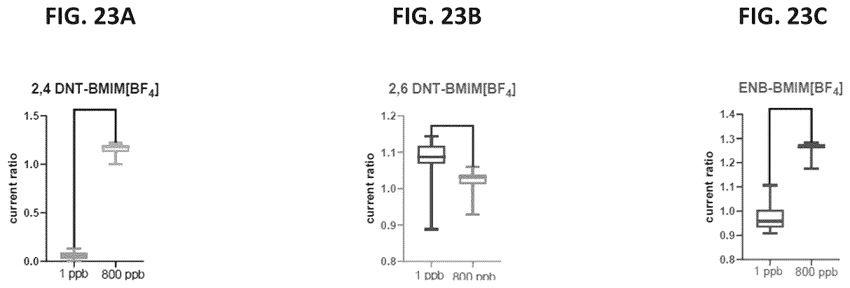

FIGS. 23A-23C show the detection of each VOC analyte at 1 ppb and 800 ppb, measured as a current ratio (detected current measured in presence of VOC/baseline current measured without presence of VOC). Specifically, 1 ppb and 800 ppb of VOC solution were prepared, and 3 uL of RTIL was dispensed on the sensor surface and baseline reading was recorded in the absence of VOC. 1 ppb VOC was added inside a sensing chamber and current response through chronoamperometry (CA) was measured. The signal was recorded, and the chamber was cleaned with N2 to remove any residual VOC before next concentration was tested. The procedure was repeated for 800 ppb VOC and the signal change (relative to 1 ppb VOC) was recorded. In each case, the sensor was able to detect the analyte at both 1 ppb and 800 ppb. Further, the sensor was able to differentiate between the highest and lowest concentrations tested. The difference in response was found to be statistically significant when analyzed using a two-tailed T-test (P values <0.0001 in all cases).

### Example 3: Sensor Calibration for Detection of COVID-19

SARS-COV-2 is a virus that has infected millions of people worldwide, causing the disease known as COVID-19. Early detection of this virus can aid in slowing the transmission in the community. COVID-19 has been associated with various respiratory diseases such as asthma, pneumonia.

Utility of a breath analyzer-based sensor platform for detection of trace amounts of target agents associated with asymptomatic and symptomatic manifestations of COVID-19 was explored. For example, an electrochemical sensor platform as described herein was used to detect the VOCs and inorganic gases released as a result of upregulation of metabolic processes in the body caused by COVID-19 and associated respiratory conditions such as asthma and pneumonia. Detection of these diseases using electrochemical sensors can help in isolation of symptomatic, asymptomatic, and/or early positive patients infected with COVID-19.

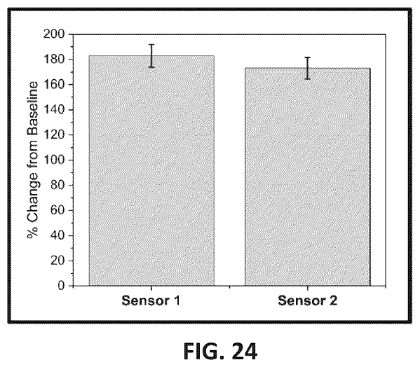

Two electrochemical sensors (Sensor 1 and Sensor 2) were characterized with a baseline study. A stable baseline current reading from each sensor was first performed in the presence of 750 PPM CO₂, which was designed to mimic health human breath. A current signal response from each sensor in the presence of a known target agent mix including NOx was then recorded to provide a calibrated response as shown in FIG. 1. For example, FIG. 24 illustrates that in response to exposure to the target agent mix, Sensor 1 detected a 182% change in current relative to its baseline reading, while Sensor 2 detected a 173% change in current relative to its baseline reading.

### Example 4: Testing for Detection of COVID-19

A detection device including two electrochemical sensors (Sensor 1, Sensor 2) was made as described above. Sensor 1 included an RTIL of 1-ethyl-3-methyl-imidazolium tetrafluoroborate (EMIM-BF₄) for detecting and characterizing NOx in breath, and Sensor 2 included an RITL of 1-ethyl-3-methyl-imidazolium trifluoromethane sulfonate (EMIM-OTf) for detecting and characterizing aliphatic carbons (e.g., isopentane, heptane) in breath. A baseline characterization was initially recorded separately to avoid any interference in readings and characterize the sensor performance prior to human subject testing. Specifically, the baseline characterization was performed using 750 PPM CO₂ (to mimic healthy human breath).

Eighteen subjects were asked to breathe in the detection device twice to record the signals for breath analytes (NOx). The sampling was performed twice to reduce any error in data collection. The change in signal relative to the baseline characterization was used as a parameter to characterize the presence or absence of the disease. Patients were selected at random and a blinded study was carried out. Multiple readings were recorded to get 95% confidence in sensor performance.

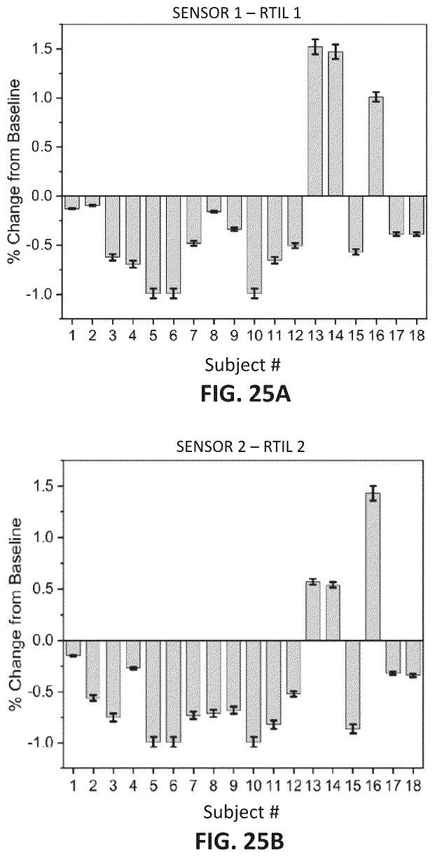

FIGS. 25A and 25B show the % change in sensor signal (current) relative to baseline for each tested subject, as measured by Sensor 1 (FIG. 25A) and Sensor 2 (FIG. 25B). A significant positive percent change relative to baseline is indicative of a presumptive positive result for COVID-19 in a subject, while little to no change (or negative change) is indicative of a healthy subject. For Subjects 1-12, and Subjects 15, 17, 18, the current obtained was less than the baseline measurement; thus the change in current is plotted as negative. However, for Subjects 13, 14, and 16, the signal response was above the baseline and was plotted as positive. The relatively large positive change from baseline for Subjects 13, 14, and 16 suggest potentially a presumptive positive result for COVID-19 in these subjects.

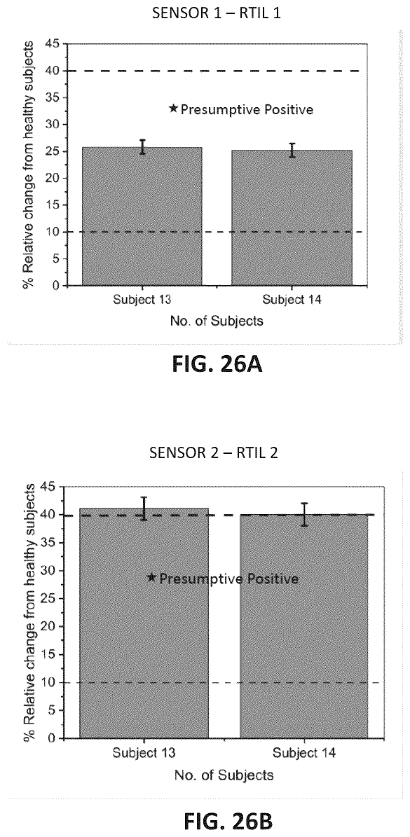

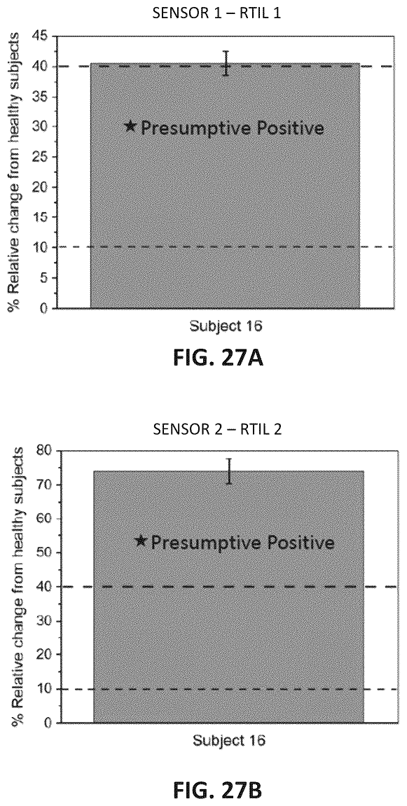

The sensor data for Subjects 13, 14, and 16 was additionally compared to an adjusted baseline characterization with the assumption that the other fifteen subjects were healthy subjects. The adjusted baseline characterization was calculated as the average sensor measurements for the other fifteen subjects. FIGS. 26A and 26B depict % change in sensor signal relative to the adjusted baseline characterization for Subjects 13 and 14, as measured using Sensor 1 (FIG. 26A) and Sensor 2 (FIG. 26B). As shown in these figures, Sensor 1 measured an approximately 25% positive change in signal for Subjects 13 and 14, while Sensor 2 measured an approximately 40% positive change in signal for Subjects 13 and 14. Similarly, FIGS. 27A and 27B depict % change in sensor signal relative to the adjusted baseline characterization for Subject 16, as measured using Sensor 1 (FIG. 27A) and Sensor 2 (FIG. 26B). As shown in these figures, Sensor 1 measured an approximately 40% positive change in signal for Subject 16, while Sensor 2 measured an approximately 75% positive change in signal for Subject 16. FIGS. 26A-26B and 27A-27B illustrate the successful use Sensors 1 and 2 in distinguishing presumptive positive COVID-19 subjects from healthy subjects.

### Example 5: Clinical Study #1for Detection of COVID-19

A breath analyzer-based detection device for detection of COVID-19 was tested on 168 patients for a total of 168 assessments. Each assessment provided an assessment result of “Detected” indicating that a signature of exhaled VOCs and inorganic gases indicative of COVID-19 infection was detected, “Not Detected” indicating that the signature of exhaled VOCs and inorganic gases indicative of COVID-19 infection was not detected, or “Defective” indicating an error in the assessment, typically due to a lack of connection between the mouthpiece and the body of the detection device. Additionally, each of the patients was tested using a conventional polymerase chain reaction (PCR) test for COVID-19 to provide an indication of actual infection status of each patient. For each of the 102 assessments, the breath analyzer-based test result was compared to the PCR test result to assess accuracy of the breath analyzer-based detection device in detecting COVID-19 in a patient.

Out of the 168 assessments, 35 positive breath analyzer-based test results were considered “true positives” that matched corresponding positive PCR test results, 37 positive breath analyzer-based test results were considered “false positives” that did not match corresponding positive PCR test results, 94 negative breath analyzer-based test results were considered “true negatives” that matched corresponding negative PCR test results, and 2 negative breath analyzer-based test results were considered “false negatives” that did not match corresponding negative PC test results. Based on these results, the breath analyzer-based detection device was found to have an accuracy of 76.8%, a specificity of 71.8%, and a sensitivity of 94.6%.

### Example 6: Clinical Study #2 for Detection of COVID-19

Three different detection devices using the same breath analyzer-based sensor platform for detection of COVID-19 were tested on 84 patients for a total of 102 assessments, where each assessment analyzed an exhaled volume of two breaths from the patient. Each assessment provided an assessment result of “Detected” indicating that a signature of exhaled VOCs and inorganic gases indicative of COVID-19 infection was detected, “Not Detected” indicating that the signature of exhaled VOCs and inorganic gases indicative of COVID-19 infection was not detected, or “Defective” indicating an error in the assessment, typically due to a lack of connection between the mouthpiece and the body of the detection device. In some instances, a “Defective” assessment for a patient was followed by a subsequent assessment to obtain either a “Detected” or “Not Detected” result for that patient. Additionally, each of the patients was tested using a conventional polymerase chain reaction (PCR) test for COVID-19 to provide an indication of actual infection status of each patient. For each of the 102 assessments, the breath analyzer-based test result was compared to the PCR test result to assess accuracy of the breath analyzer-based detection device in detecting COVID-19 in a patient.

Out of the 102 assessments, 21 assessments were considered defective due to user error. Among the other assessments, 27 positive breath analyzer-based test results were considered “true positives” that matched corresponding positive PCR test results, 7 positive breath analyzer-based test results were considered “false positives” that did not match corresponding positive PCR test results, 47 negative breath analyzer-based test results were considered “true negatives” that matched corresponding negative PCR test results, and 0 negative breath analyzer-based test results were considered “false negatives” that did not match corresponding negative PCR test results. Based on these results, the breath analyzer-based platform was found to have a high degree of sensitivity (100%), a high degree of specificity (87.0%), and a high degree of accuracy (91.4%).

### ENUMERATED EMBODIMENTS

Embodiment 1. A detection device for detecting one or more volatile organic compounds (VOCs), the detection device comprising:

a base; and

a sensor module removably coupleable to the base and comprising at least one electrochemical sensor,

wherein the at least one electrochemical sensor comprises an electrode and an ionic liquid that is arranged on the electrode and specific to a target VOC.

Embodiment 2. The detection device of embodiment 1, wherein the ionic liquid comprises a room temperature ionic liquid (RTIL).

Embodiment 3. The detection device of embodiment 2, wherein the ionic liquid comprises a plurality of ionic layers, wherein at least one cavity specific to the target VOC is formed between adjacent ionic layers in response to an input signal provided to the electrochemical sensor.

Embodiment 4. The detection device of embodiment 3, wherein the detection device is configured to deliver the input signal to the electrochemical sensor.

Embodiment 5. The detection device of embodiment 4, wherein the input signal applies a DC reduction potential to the electrode.

Embodiment 6. The detection device of embodiment 3, wherein the at least one cavity is configured to capture the target VOC such that the captured VOC diffuses toward the electrode.

Embodiment 7. The detection device of embodiment 5, wherein the base comprises one or more processors configured to detect the captured target VOC based at least in part on impedance, current, or both at the electrode.

Embodiment 8. The detection device of embodiment 1, wherein the base comprises an alarm configured to provide an alert in response to detection of the target VOC using the at least one electrochemical sensor.

Embodiment 9. The detection device of embodiment 1, wherein the base comprises a wireless communication module.

Embodiment 10. The detection device of embodiment 1, wherein the base comprises a handheld housing.

Embodiment 11. The detection device of embodiment 1, wherein the base is configured to be mounted to a surface.

Embodiment 12. The detection device of embodiment 1, wherein the sensor module comprises a plurality of electrochemical sensors.

Embodiment 13. The detection device of embodiment 12, wherein each of at least a portion of the plurality of electrochemical sensors comprises a respective ionic liquid, wherein the respective ionic liquids are specific to the same target VOC.

Embodiment 14. The detection device of embodiment 12, wherein each of at least a portion of the plurality of electrochemical sensors comprises a respective ionic layer, wherein the respective ionic layers are specific to different target VOCs.

Embodiment 15. The detection device of embodiment 1, wherein the sensor module comprises one or more electrical contacts configured to conductively couple to the base.

Embodiment 16. The detection device of embodiment 1, wherein the sensor module comprises a mouthpiece.

Embodiment 17. The detection device of embodiment 1, wherein the target VOC is characteristic of an explosive.

Embodiment 18. The detection device of embodiment 1, wherein the target VOC is characteristic of a drug.

Embodiment 19. The detection device of embodiment 1, wherein the target VOC is a biomarker characteristic of a health state of a user.

Embodiment 20. An electrochemical sensor for use in detecting a target volatile organic compound (VOC), the electrochemical sensor comprising:

an electrode;

a room temperature ionic liquid (RTIL) arranged over the electrode;

wherein at least one cavity specific to the target VOC is formed within the RTIL in response to the sensor receiving an input signal.

Embodiment 21. The sensor of embodiment 20, wherein the electrode comprises gold.

Embodiment 22. The sensor of embodiment 21, wherein the electrode comprises an interdigitated electrode.

Embodiment 23. The electrochemical sensor of embodiment 20, wherein the RTIL is selected from the group consisting of: 1-butyl-3-methylimidazolium chloride; 1-butyl-3-methylimidazolium tetrafluoroborate; 1-ethyl-3-methylimidazolium bis-(trifluoromethanesulphonyl)imide; 1-ethyl-3-methylimidazolium tetrafluoroborate; and 1-ethyl-3-methylimidazolium trifluromethanesulfonate.

Embodiment 24. The electrochemical sensor of embodiment 20, wherein the RTIL comprises a plurality of ionic layers.

Embodiment 25. The electrochemical sensor of embodiment 20, wherein the RTIL comprises at least 2 ionic layers.

Embodiment 26. The sensor of any one of embodiments 24-25, wherein the at least one cavity is formed between adjacent ionic layers.

Embodiment 27. The electrochemical sensor of embodiment 20, wherein the input signal applies a DC reduction potential to the electrode.

Embodiment 28. The electrochemical sensor of embodiment 27, wherein the input signal corresponds to a redox potential of the target VOC.

Embodiment 29. The electrochemical sensor of embodiment 28, wherein the at least one cavity has a size corresponding to the redox potential of the target VOC.

Embodiment 30. The electrochemical sensor of embodiment 29, wherein the at least one cavity is configured to capture the target VOC such that the captured target VOC diffuses toward the electrode.

Embodiment 31. The electrochemical sensor of embodiment 20, wherein the target VOC is characteristic of an explosive.

Embodiment 32. The electrochemical sensor of embodiment 31, wherein the target VOC characteristic of an explosive is selected from the group consisting of: 1,3-dinitrobenzene; 2,4-dinitrotoluene; 2,6-dinitrotoluene; 1-ethyl-2-nitrobenzene; 2,3-dim ethyl-2,3-dinitrobutane; sulfur dioxide; and cyclohexanone.

Embodiment 33. The electrochemical sensor of embodiment 32, wherein the target VOC is characteristic of C-4.

Embodiment 34. The electrochemical sensor of embodiment 32, wherein the target VOC is characteristic of gunpowder.

Embodiment 35. The electrochemical sensor of embodiment 20, wherein the target VOC is a biomarker associated with a medical condition.

Embodiment 36. The electrochemical sensor of embodiment 35, wherein the biomarker associated with a medical condition is NOx or an aliphatic hydrocarbon.

Embodiment 37. The electrochemical sensor of embodiment 20, wherein the target VOC is associated with use of a drug.

Embodiment 38. The electrochemical sensor of embodiment 37, wherein the drug is an opioid.

Embodiment 39. The electrochemical sensor of embodiment 38, wherein the opioid is fentanyl.

Embodiment 40. The use of an electrochemical sensor according to any of embodiments 29-34 to detect presence of a nearby explosive.

Embodiment 41. The use of an electrochemical sensor according to any of embodiments 20-30 or 35-36 to detect presence of a health state of a user.

Embodiment 42. The use of embodiment 41, wherein the health state is a disease.

Embodiment 43. The use of embodiment 41, wherein the disease is COVID-19.

Embodiment 44. A detection device comprising the electrochemical sensor of any of embodiments 20-43.

Embodiment 45. A method for detecting one or more volatile organic compounds (VOCs), the method comprising:

applying an input signal to an electrochemical sensor, the electrochemical sensor comprising an electrode and an ionic liquid arranged over the electrode, wherein at least one cavity specific to a target VOC is formed within the ionic liquid in response to the input signal;

receiving a sensor signal from the electrochemical sensor after applying the input signal; and

detecting the target VOC based at least in part on the sensor signal.

Embodiment 46. The method of embodiment 45, wherein the ionic liquid comprises a room temperature ionic liquid (RTIL).

Embodiment 47. The method of embodiment 45, wherein the sensor signal comprises current at the electrode.

Embodiment 48. The method of embodiment 45, wherein the at least one cavity is tuned to the redox potential of the target VOC.

Embodiment 49. The method of embodiment 45, comprising applying an input signal to a plurality of electrochemical sensors, each comprising a respective electrode and respective ionic liquid arranged over the electrode.

Embodiment 50. The method of embodiment 49, wherein in response to the input signal, the respective ionic liquids of at least a portion of the plurality of electrochemical sensors form cavities that are specific to the same target VOC.

Embodiment 51. The method of embodiment 50, wherein detecting the target VOC comprises sensing the target VOC using a majority of the electrochemical sensors specific to the target VOC.

Embodiment 52. The method of embodiment 50, further comprising determining at least one of travel direction and travel speed of the target VOC, based on differential timing of detection of the target VOC using the electrochemical sensors specific to the target VOC.

Embodiment 53. The method of embodiment 49, wherein in response to the input signal, the respective ionic liquids of at least a portion of the plurality of electrochemical sensors form cavities that are specific to different target VOCs.

Embodiment 54. The method of embodiment 45, further comprising providing an alert in response to detection of the target VOC.

Embodiment 55. The method of embodiment 45, wherein the target VOC has a concentration gradient, and wherein detecting the target VOC comprises distinguishing the target VOC from other gases having the same concentration gradient as the target VOC.

Embodiment 56. The method of embodiment 45, wherein the target VOC is characteristic of an explosive.

Embodiment 57. The method of embodiment 45, wherein the target VOC is characteristic of a drug.

Embodiment 58. The method of embodiment 45, wherein the target VOC is a biomarker characteristic of a health state of a user.

Embodiment 59. The method of embodiment 45, wherein the target VOC is emitted from a solid medium.

Embodiment 60. The method of embodiment 45, wherein the target VOC is emitted from a liquid medium.

Embodiment 61. The method of embodiment 45, wherein the target VOC is emitted from a gas medium.

Embodiment 62. A method for determining a health state of a user, the method comprising:

measuring a sensor signal of at least one electrochemical sensor receiving an aerosolized sample, the at least one electrochemical sensor comprising an electrode and a room temperature ionic liquid (RTIL) that is arranged on the electrode, wherein at least one cavity specific to a target volatile organic compound (VOC) is formed within the RTIL in response to the electrochemical sensor receiving an input signal;

detecting the target VOC based at least in part on the measured sensor signal; and

determining the health state of the user based on the detected target VOC.

Embodiment 63. The method of embodiment 62, wherein the RTIL comprises a plurality of ionic layers and the at least one cavity is formed between adjacent ionic layers.

Embodiment 64. The method of embodiment 63, measuring a sensor signal comprises delivering an input signal to the at least one electrochemical sensor and measuring impedance, current, or both, at the at least one electrochemical sensor after delivering the input signal.

Embodiment 65. The method of embodiment 64, wherein the input signal applies a DC reduction potential to the electrode.

Embodiment 66. The method of embodiment 62, wherein the at least one cavity is configured to capture the target VOC such that the captured target VOC diffuses toward the electrode.

Embodiment 67. The method of embodiment 62, further comprising providing an alert in response to detection of the health state.

Embodiment 68. The method of embodiment 62, wherein detecting the target VOC comprises detecting the target VOC in an aerosolized sample.

Embodiment 69. The method of embodiment 68, further comprising filtering the aerosolized sample to remove particulates above a threshold size.

Embodiment 70. The method of embodiment 68, wherein the aerosolized sample comprises breath from the user.

Embodiment 71. The method of embodiment 68, wherein the aerosolized sample comprises an aerosolized sample of body fluid.

Embodiment 72. The method of embodiment 71, wherein the body fluid comprises at least one of saliva and nasal fluid.

Embodiment 73. The method of embodiment 71, wherein the aerosolized sample is from a sampling device.

Embodiment 74. The method of embodiment 68, wherein the aerosolized sample is from ambient air.

Embodiment 75. The method of embodiment 62, wherein the at least one electrochemical sensor is in a sensor module removably coupled to a base.

Embodiment 76. The method of embodiment 75, wherein the base comprises a handheld unit.

Embodiment 77. The method of embodiment 75, wherein the base is configured to be mounted to a surface.

Embodiment 78. The method of embodiment 75, wherein the sensor module comprises a mouthpiece and a nozzle configured to provide for laminar flow of the aerosolized sample over the at least one electrochemical sensor.

Embodiment 79. The method of embodiment 62, wherein the target VOC is a biomarker characteristic of a disease.

Embodiment 80. The method of embodiment 79, wherein the disease is COVID-19.

Embodiment 81. A detection device for detecting one or more volatile organic compounds (VOCs) in breath of a user, the detection device comprising:

a base; and

a sensor module removably coupled to the base, wherein the sensor module comprises:

at least one electrochemical sensor comprising an electrode and an ionic liquid arranged over the electrode, wherein the ionic liquid is specific to a target VOC;

a mouthpiece configured to direct a volume of breath from the user toward the at least one electrochemical sensor.

Embodiment 82. The detection device of embodiment 81, wherein the ionic liquid comprises a room temperature ionic liquid (RTIL).

Embodiment 83. The detection device of embodiment 81, wherein the base comprises a handheld housing.

Embodiment 84. The detection device of embodiment 81, wherein the detection device is configured to deliver an input signal to the electrochemical sensor, thereby forming at least one cavity specific to the target VOC within the ionic liquid.

Embodiment 85. The detection device of embodiment 84, wherein the at least one cavity is configured to capture the target VOC such that the captured VOC diffuses toward the electrode.

Embodiment 86. The detection device of embodiment 85, wherein the base comprises one or more processors configured to detect the captured target VOC based at least in part on impedance, current, or both, at the electrode.

Embodiment 87. The detection device of embodiment 81, wherein the base comprises an alarm configured to provide an alert in response to detection of the target VOC using the at least one electrochemical sensor.

Embodiment 88. The detection device of embodiment 87, wherein the sensor module comprises a plurality of electrochemical sensors.

Embodiment 89. The detection device of embodiment 88, wherein each of at least a portion of the plurality of electrochemical sensors comprises a respective ionic liquid, wherein the respective ionic liquids are specific to the same target VOC.

Embodiment 90. The detection device of embodiment 88, wherein each of at least a portion of the plurality of electrochemical sensors comprises a respective ionic layer, wherein the respective ionic layers are specific to different target VOCs.

Embodiment 91. The detection device of embodiment 81, wherein the mouthpiece comprises a tube.

Embodiment 92. The detection device of embodiment 82, wherein the sensor module comprises a nozzle configured to laminarize flow of the volume of breath over the at least one electrochemical sensor.

Embodiment 93. The detection device of embodiment 81, wherein the sensor module comprises one or more filters configured to filter particulates from the volume of breath.

Embodiment 94. The detection device of embodiment 81, wherein the sensor module comprises one or more dehumidifying elements configured to reduce moisture in the volume of breath.

Embodiment 95. The detection device of embodiment 81, wherein the target analyte is a biomarker characteristic of a health state of the user.

Embodiment 96. The detection device of embodiment 95, wherein the health state is a disease.

Embodiment 97. The detection device of embodiment 96, wherein the disease is COVID-19.

Embodiment 98. A detection system for detecting one or more volatile organic compounds (VOCs) in breath of a user, the detection system comprising:

a sensor module comprising at least one electrochemical sensor specific to a target VOC; and

a sampling device coupleable to the sensor module, wherein the sampling device is sealable and configured to store a volume of breath.

Embodiment 99. The detection system of embodiment 98, wherein the sensor module comprises an electrode and an ionic liquid arranged over the electrode, wherein the ionic liquid is specific to the target VOC.

Embodiment 100. The detection system of embodiment 98, wherein the sampling device is removably coupleable to the sensor module.

Embodiment 101. The detection system of embodiment 98, wherein the sampling device is coupleable to the sensor module via a connector.

Embodiment 102. The detection system of embodiment 98, wherein the sampling device comprises a compartment.

Embodiment 103. The detection system of embodiment 102, wherein the compartment is compressible.

Embodiment 104. The detection system of embodiment 98, wherein the sampling device comprises a mouthpiece.

Embodiment 105. The detection system of embodiment 104, wherein the mouthpiece comprises one or more filters.

Embodiment 106. The detection system of embodiment 104, wherein the mouthpiece comprises a desiccant.

Embodiment 107. The detection system of embodiment 104, wherein the sampling device comprises one or more one-way valves.

Embodiment 108. The detection system of embodiment 98, further comprising a base, wherein the sensor module is coupleable to the base.

Embodiment 109. The detection system of embodiment 108, wherein the sensor module is removably coupleable to the base.

Embodiment 110. The detection system of embodiment 109, wherein the base comprises a handheld housing.

Embodiment 111. The detection system of embodiment 98, further comprising an alarm configured to provide an alert in response to detection of the target VOC using the at least one electrochemical sensor.

Embodiment 112. A sampling device comprising:

a compartment; and

a mouthpiece coupled to the compartment,

wherein the sampling device is sealable and configured to store a volume of a gas sample.

Embodiment 113. The sampling device of embodiment 112, wherein the compartment comprises an inlet and an outlet.

Embodiment 114. The sampling device of embodiment 113, wherein the mouthpiece is coupled to the inlet of the compartment, and wherein the sampling device further comprises a stopper coupled to the outlet of the compartment.

Embodiment 115. The sampling device of embodiment 114, wherein the stopper is removably coupled to the outlet of the compartment.

Embodiment 116. The sampling device of embodiment 112, wherein the sampling device is sealable via one or more one-way valves.

Embodiment 117. The sampling device of embodiment 116, wherein the sampling device comprises an inlet sealable with a first one-way valve, and an outlet sealable with a second one-way valve.

Embodiment 118. The sampling device of embodiment 116, wherein the one or more one-way valves comprises a check valve.

Embodiment 119. The sampling device of embodiment 112, wherein the compartment is compressible.

Embodiment 120. The sampling device of embodiment 119, wherein the compartment comprises a bag.

Embodiment 121. The sampling device of embodiment 120, wherein the bag comprises a first sheet and a second sheet opposing the first sheet, wherein the first and second sheets are sealed together to form an edge of the compartment.

Embodiment 122. The sampling device of embodiment 112, wherein the mouthpiece comprises a tube.

Embodiment 123. The sampling device of embodiment 112, wherein the mouthpiece comprises one or more filters.

Embodiment 124. The sampling device of embodiment 112, wherein the mouthpiece comprises a desiccant.

Embodiment 125. The sampling device of embodiment 112, wherein the mouthpiece is RF or heat welded to the compartment.

Embodiment 126. The sampling device of embodiment 112, wherein the sampling device is configured to removably couple to a detection device.

Embodiment 127. The sampling device of embodiment 112, further comprising a labeling region.

Embodiment 128. The sampling device of embodiment 112, further comprising a computer-readable identifier associated with the sampling device.

Embodiment 129. A detection device for detecting one or more volatile organic compounds (VOCs) in breath of a user, the detection device comprising:

a sensor module comprising at least one electrochemical sensor comprising an electrode and an ionic liquid arranged over the electrode, wherein the ionic liquid is specific to a target VOC; and

a mouthpiece configured to direct a volume of breath from the user toward the at least one electrochemical sensor.

Embodiment 130. The detection device of embodiment 129, wherein the ionic liquid comprises a room temperature ionic liquid (RTIL).

Embodiment 131. The detection device of embodiment 129, wherein the detection device comprises a handheld housing and wherein the sensor module is arranged in the handheld housing.

Embodiment 132. The detection device of embodiment 129, wherein the detection device is configured to deliver an input signal to the electrochemical sensor, thereby forming at least one cavity specific to the target VOC within the ionic liquid.

Embodiment 133. The detection device of embodiment 132, wherein the at least one cavity is configured to capture the target VOC such that the captured VOC diffuses toward the electrode.

Embodiment 134. The detection device of embodiment 133, further comprising one or more processors configured to detect the captured target VOC based at least in part on impedance, current, or both, at the electrode.

Embodiment 135. The detection device of embodiment 129, further comprising an alarm configured to provide an alert in response to detection of the target VOC using the at least one electrochemical sensor.

Embodiment 136. The detection device of embodiment 129, wherein the sensor module comprises a plurality of electrochemical sensors.

Embodiment 137. The detection device of embodiment 136, wherein each of at least a portion of the plurality of electrochemical sensors comprises a respective ionic liquid, wherein the respective ionic liquids are specific to the same target VOC.

Embodiment 138. The detection device of embodiment 136, wherein each of at least a portion of the plurality of electrochemical sensors comprises a respective ionic layer, wherein the respective ionic layers are specific to different target VOCs.

Embodiment 139. The detection device of embodiment 129, wherein the mouthpiece comprises a tube.

Embodiment 140. The detection device of embodiment 129, wherein the mouthpiece comprises one or more filters configured to filter particulates from the volume of breath.

Embodiment 141. The detection device of embodiment 129, wherein the mouthpiece comprises one or more dehumidifying elements configured to reduce moisture in the volume of breath.

Embodiment 142. The detection device of embodiment 129, wherein the mouthpiece is coupled to a sampling device coupleable to the sensor module, wherein the sampling device is sealable and configured to store the volume of breath.

Embodiment 143. The detection device of embodiment 142, wherein the sampling device is removably coupleable to the sensor module.

Embodiment 144. The detection device of embodiment 142, wherein the sampling device comprises a compressible compartment.

Embodiment 145. The detection device of embodiment 142, wherein the sampling device comprises one or more one-way valves.

Embodiment 146. The detection device of embodiment 129, wherein the target VOC is a biomarker characteristic of a health state of the user.

Embodiment 147. The detection device of embodiment 146, wherein the health state is a disease.

Embodiment 148. The detection device of embodiment 147, wherein the disease is COVID-19.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to explain the principles of the invention and its practical applications, they thereby enable others skilled in the art to utilize the invention and various embodiments with various modifications as are suited to the particular use contemplated. It is intended that the following claims and their equivalents define the scope of the invention.

1.-20. (canceled)

21: A detection device for detecting one or more volatile organic compounds (VOCs) in breath of a user, the detection device comprising:
a sensor module comprising at least one electrochemical sensor comprising an electrode and an ionic liquid arranged over the electrode, wherein the ionic liquid comprises a 1-ethyl-3-methyl-imidazolium salt and is specific to a target VOC characteristic of COVID-19; and
a mouthpiece configured to direct a volume of breath from the user toward the at least one electrochemical sensor.

22: The detection device of claim 21, wherein the ionic liquid comprises a room temperature ionic liquid (RTIL).

23: The detection device of claim 21, wherein the detection device comprises a handheld housing and wherein the sensor module is arranged in the handheld housing.

24: The detection device of claim 21, wherein the detection device is configured to deliver an input signal to the electrochemical sensor, thereby forming at least one cavity specific to the target VOC within the ionic liquid.

25: The detection device of claim 24, wherein the at least one cavity is configured to capture the target VOC such that the captured VOC diffuses toward the electrode.

26: The detection device of claim 25, further comprising one or more processors configured to detect the captured target VOC based at least in part on impedance, current, or both, at the electrode.

27: The detection device of claim 21, further comprising an alarm configured to provide an alert in response to detection of the target VOC using the at least one electrochemical sensor.

28: The detection device of claim 21, wherein the sensor module comprises a plurality of electrochemical sensors.

29: The detection device of claim 28, wherein each of at least a portion of the plurality of electrochemical sensors comprises a respective ionic liquid, wherein the respective ionic liquids are specific to the same target VOC.

30: The detection device of claim 28, wherein each of at least a portion of the plurality of electrochemical sensors comprises a respective ionic layer, wherein the respective ionic layers are specific to different target VOCs.

31: The detection device of claim 21, wherein the mouthpiece comprises a tube.

32: The detection device of claim 21, wherein the mouthpiece comprises one or more filters configured to filter particulates from the volume of breath.

33: The detection device of claim 21, wherein the mouthpiece comprises one or more dessicants configured to reduce moisture in the volume of breath.

34: The detection device of claim 21, wherein the mouthpiece is coupled to a sampling device coupleable to the sensor module, wherein the sampling device comprises a sealable compartment configured to store the volume of breath.

35: The detection device of claim 34, wherein the sampling device is removably coupleable to the sensor module.

36: The detection device of claim 34, wherein the compartment of the sampling device is compressible.

37: The detection device of claim 34, wherein the sampling device comprises one or more one-way valves.

38: The device of claim 21, wherein the ionic liquid comprises 1-ethyl-3-methyl-imidazolium tetrafluoroborate.

39: The device of claim 21, wherein the ionic liquid comprises 1-ethyl-3-methyl-imidazolium trifluoromethane sulfonate.
